(19) United States
(12) Patent Application Publication
(10) Pub. No.: US 2022/0313929 A1
(43) Pub. Date: Oct. 6, 2022

(54) CLOSED LOOP CONTROL IN MECHANICAL VENTILATION

(71) Applicant: ZOLL Medical Corporation, Chelmsford, MA (US)

(72) Inventors: George Beck, Salem, MA (US); Brian P. Harvey, Belmont, MA (US); Dorian LeCroy, New York, NY (US)

(21) Appl. No.: 17/656,936

(22) Filed: Mar. 29, 2022

Related U.S. Application Data
(60) Provisional application No. 63/167,886, filed on Mar. 30, 2021.

(51) Int. Cl. A61M 16/00; A61B 5/08

(52) U.S. Cl. CPC A61M 16/0003; A61B 5/0816; A61M 16/06

(57) ABSTRACT

Apparatus, systems and methods are described, such as for providing, or controlling mechanical ventilation provided to, a patient. A controller may control a gas delivery system to deliver gas to the patient according to a FiO2 setting and a PEEP setting. The controller may adjust the FiO2 setting to an updated FiO2 setting based at least in part on a determined oxygen concentration of the patient's blood and may update the PEEP setting based at least in part on the updated FiO2 setting. Furthermore, the controller may update the PEEP setting based at least in part on the updated FiO2 setting and the current PEEP setting. An updated PEEP setting may be based at least in part on PEEP change eligibility rules and PEEP selection rules. The FiO2 setting may be adjusted so as to relatively rapidly increase the FiO2 setting in response to a rapidly decreasing patient SpO2.

### BACKGROUND

Providing mechanical ventilation to individuals, when appropriate, as well as optimizing aspects of the provided ventilation, can pose critical challenges, particularly in pre-hospital or other non-hospital settings, among others. In non-hospital settings, for example, care providers may have limited experience or training relating to ventilation, which can exacerbate the problem. Yet optimizing ventilation, which can include tracking and continuous adjustment of a number of ventilation related parameters, can be crucial to the patient's care and even survival.

Given the foregoing, it is perhaps unsurprising that, unfortunately, suboptimal or injurious ventilation practices and patterns have been common, particularly in non-hospital or pre-hospital settings where caregivers who are inexperienced in respiratory therapy are tasked with providing ventilation to the patient. These may include, for example, suboptimal, inappropriate or injurious tidal volume, positive end-expiratory pressure (PEEP) and patient oxygenation, among other things. As such, for example, providing or facilitating providing safe, optimized mechanical ventilation, particularly (although not only) in non-hospital and pre-hospital settings and with less intensely ventilation-trained care providers, has remained challenging.

### SUMMARY

An example, according to some embodiments of the disclosure, of a mechanical ventilator apparatus includes: a gas delivery apparatus, having a patient interface, configured to deliver gas to a patient; an oximetry sensor configured to generate signals representative of an oxygen concentration of the patient's blood; and a controller, including a processor and a memory, in communication with the gas delivery apparatus and the oximetry sensor, the controller being configured to: control the gas delivery apparatus to deliver the gas to the patient according to a FiO2 setting and a PEEP setting, wherein the FiO2 setting and the PEEP setting are configured to be adjustable, control the delivery of the gas to the patient according to a first FiO2 value and a first PEEP value, receive the signals representative of the oxygen concentration of the patient's blood from the oximetry sensor during the delivery of the gas to the patient, determine the oxygen concentration of the patient's blood based at least in part on the received signals, based at least in part on the oxygen concentration of the patient's blood, control the gas delivery apparatus to adjust the FiO2 setting to an updated FiO2 value, and based at least in part on the adjustment to the FiO2 setting, control the gas delivery apparatus to adjust the PEEP setting to an updated PEEP value.

An example, according to some embodiments of the disclosure, of a mechanical ventilator apparatus includes: a gas delivery apparatus, having a patient interface, configured to deliver gas to a patient; an oximetry sensor configured to generate signals representative of an oxygen concentration of the patient's blood; and a controller, including a processor and a memory, in communication with the gas delivery apparatus and the oximetry sensor, the controller being configured to: control the gas delivery apparatus to deliver the gas to the patient according to a FiO2 setting and a PEEP setting, wherein the FiO2 setting and the PEEP setting are configured to be adjustable, control the delivery of the gas to the patient according to a first FiO2 value and a first PEEP value, receive the signals representative of the oxygen concentration of the patient's blood from the oximetry sensor during the delivery of the gas to the patient, determine the oxygen concentration of the patient's blood based at least in part on the received signals, based at least in part on the oxygen concentration of the patient's blood, control the gas delivery apparatus to adjust the FiO2 setting to an updated FiO2 setting, and based at least in part on the updated FiO2 setting, control the gas delivery apparatus to adjust the PEEP setting to an updated PEEP value.

Some implementations may include one or more of the following features. According to some embodiments, the oximetry sensor may include a pulse oximetry sensor, which may include an SpO2 sensor. The oxygen concentration of the patient's blood may be an oxygen saturation. The gas may be a breathing gas. An updated PEEP value may be determined based at least in part on an updated FiO2 setting and a first PEEP value.

The adjustment to the FiO2 setting may include an adjustment to the FiO2 setting from a first FiO2 level to a second FiO2 level, wherein the adjustment to the PEEP setting includes an adjustment in the PEEP setting from a first PEEP level to a second PEEP level, and wherein the determined PEEP update is based at least in part on: the second FiO2 level, and the first PEEP level.

The PEEP setting may be adjusted based on a selection from at least two PEEP levels including a first PEEP level associated with a first FiO2 range and a second PEEP level associated with a second FiO2 range, wherein the first FiO2 range overlaps with the second FiO2 range, wherein the PEEP update is determined so as to differ from the PEEP setting if one or more conditions are met, wherein the one or more conditions include that a level of FiO2 of the gas being delivered to the patient has changed so as to fall outside of the first FiO2 range. The adjustment to the PEEP setting may include a change in the PEEP setting from the first PEEP level to the second PEEP level.

A change in the PEEP setting may be based on a set of one or more PEEP change eligibility conditions being met, the set of conditions including that: the FiO2 setting has not changed by at least a first amount in at least a first specified period of time or the level of SpO2 of the patient has been below a desaturation threshold for more than a second specified period of time. The set of conditions may include: if the determined PEEP update includes an increase in PEEP, the PEEP setting has not changed over a third period of time, and if the determined PEEP update includes a decrease in PEEP, the PEEP setting has not changed over a fourth period of time, the third period of time being different than the fourth period of time. The fourth period of time may be greater than the third period of time.

A change in the PEEP setting may be based at least in part on a set of one or more PEEP change eligibility conditions being met, the set of conditions including that: if the determined PEEP update includes an increase in PEEP, the PEEP setting has not changed over a first period of time, and if the determined PEEP update includes a decrease in PEEP, the PEEP setting has not changed over a second period of time. The second period of time may be different than the first period of time or may be greater than the first period of time. If the determined PEEP update includes the increase in PEEP, one or more measures of a hemodynamic status of the patient may indicate that the hemodynamic status of the patient is above a first threshold.

A change the PEEP setting may be based on a set of one or more PEEP change eligibility conditions being met, the set of conditions including that: if the determined PEEP update includes an increase in PEEP, one or more measures of a hemodynamic status of the patient indicate that the hemodynamic status of the patient is above a first threshold.

The controller may be configured to estimate, assume or use a placeholder respiratory system compliance (Crs) of the patient and update a peak inspiratory pressure (PIP) setting of the ventilator apparatus based at least in part on the estimated Crs of the patient. The Crs of the patient may be estimated based at least in part on application of at least one data fitting algorithm to a set of waveforms associated with respiratory mechanics of one or more breaths administered to the patient.

The controller may be configured to, based at least in part on a determination that a driving pressure or plateau pressure being delivered to the patient is over a threshold, decrease a tidal volume (Vt) delivered to the patient. The controller may further be configured to, based at least in part on the decreased Vt delivered to the patient, increase a respiratory rate (RR) delivered to the patient, such as to maintain a steady Ve. The controller may further be configured to, based at least in part on a Vt being delivered to the patient that is below a Vt threshold, trigger a low Vt alarm. The controller may further be configured to, based at least in part on a capnographic measure that is above a threshold, increase a Ve being delivered to the patient. The capnographic measure may be an EtCO2 measure. The controller may be configured to, based at least in part on a capnographic measure that is below a threshold, reduce a PIP, Vt or Ve being delivered to the patient.

The controller may be configured to, based at least in part on a predicted or ideal bodyweight of the patient determined based at least in part a gender and a height of the patient, determine a set of initial ventilation parameters for the one or more breaths administered to the patient, the initial ventilation parameters including an initial Vt setting or an initial PIP setting used for the one or more breaths administered to the patient.

A minimum PEEP setting of the gas delivery apparatus may be between 0 cm of water (H2O) and 10 cm of water (H2O), between 1 cm of water (H2O) and 10 cm of water (H2O), such as 5 cm H2O. A maximum setting may be between 10 cm H2O and 20 cm H2O, such as 15 cm H2O.

An example, according to some embodiments of the disclosure, of a method for controlling mechanical ventilation being provided to a patient includes a controller: controlling a gas delivery system of a mechanical ventilator to deliver gas to the patient according to an FiO2 setting and a PEEP setting, wherein the FiO2 setting and the PEEP setting are adjustable; controlling the delivery of the gas to the patient according to a first FiO2 value and a first PEEP value; receiving signals representative of an oxygen concentration of the patient's blood from an oximetry sensor of the mechanical ventilator during the delivery of the gas to the patient, the oximetry sensor being coupled with the gas delivery system; determining the oxygen concentration of the patient's blood based at least in part on the received signals, based at least in part on the determined oxygen concentration of the patient's blood, controlling the gas delivery system to adjust the FiO2 setting to an updated FiO2 setting, and based at least in part on the updated FiO2 setting, controlling the gas delivery apparatus to adjust the PEEP setting to an updated PEEP value.

Some implementations may include one or more of the following features. Some embodiments include, based at least in part on the determined oxygen concentration of the patient's blood, controlling the gas delivery system to adjust the FiO2 setting to the updated FiO2 setting at least in part by actuating an oxygen source valve. Furthermore, some embodiments include, based at least in part on the updated FiO2 setting, controlling the gas delivery apparatus to adjust the PEEP setting to the updated PEEP value at least in part by actuating an exhalation valve.

An example, according to some embodiments of the disclosure, of a system for providing mechanical ventilation to a patient includes: a gas delivery system for delivering gas to a patient, including: an oximetry sensor for generating signals representative of an oxygen concentration of the patient's blood; a mechanical gas mover; an oxygen source; a patient interface coupled with the mechanical gas mover and the oxygen source; and a controller, coupled with the oximetry sensor and the compressor, for controlling the gas delivery system to deliver gas to the patient according to a FiO2 setting and a PEEP setting, wherein the FiO2 setting and the PEEP setting are configured to be adjustable, the controlling of the gas delivery system including: receiving the signals representative of the oxygen concentration of the patient's blood from the oximetry sensor during the delivery of the gas to the patient, determining the oxygen concentration of the patient's blood based at least in part on the received signals, based at least in part on the determined oxygen concentration of the patient's blood, controlling the gas delivery system to adjust the FiO2 setting to an updated FiO2 setting, including actuating at least one oxygen source valve according to the updated FiO2 setting, the at least one oxygen source valve being coupled with, and for adjusting gas flow from, the oxygen source, and based at least in part on the updated FiO2 setting, control the gas delivery system to adjust the PEEP setting to an updated PEEP setting, including actuating at least one exhalation valve according to the updated PEEP setting, the at least one exhalation valve being coupled with the compressor and the patient interface.

An example, according to some embodiments of the disclosure, of a mechanical ventilator apparatus includes: a gas delivery apparatus, having a patient interface, configured to deliver gas to a patient; and a controller, including a processor and a memory, in communication with the gas delivery apparatus, the controller being configured to: control the gas delivery apparatus to deliver the gas to the patient according to a FiO2 setting and a PEEP setting, wherein the FiO2 setting and the PEEP setting are configured to be adjustable, control the delivery of the gas to the patient according to a first FiO2 value and a first PEEP value, based at least in part on the first FiO2 value, determine an updated PEEP value selected from at least two PEEP values including the first PEEP value associated with a first FiO2 range and a second PEEP value associated with a second FiO2 range, wherein the second FiO2 range overlaps with the first FiO2 range, wherein the updated PEEP value is determined so as to differ from the first PEEP value if one or more PEEP change conditions are met, wherein the one or more PEEP change conditions include that a F102 value for the FiO2 setting has changed so as to fall outside of the first FiO2 range, and control the gas delivery apparatus to adjust the PEEP setting to the updated PEEP value.

Some implementations may include one or more of the following features. The adjustment to the PEEP setting may include a change in the PEEP setting from a first PEEP level to a second PEEP level. The first FiO2 value may be inside of the first FiO2 range. The updated PEEP value may be determined based at least in part on the first FiO2 value and the first PEEP value.

The at least two PEEP values may include at least three PEEP values including a third PEEP value associated with a third FiO2 range, wherein the third FiO2 range overlaps with the second FiO2 range, and wherein, for the PEEP setting to be adjusted so as to be changed to a different PEEP setting, the FiO2 setting is changed so as to fall outside of one of the FiO2 ranges associated with one of the at least three PEEP values.

The mechanical ventilator apparatus may include an oxygen saturation (SpO2) sensor configured to generate signals representative of oxygen saturation of the patient, wherein the controller is configured to: receive the generated signals representative of oxygen saturation, determine a level of SpO2 of the patient based on the received signals, based at least in part on the level of SpO2 of the patient, determine a FiO2 update for the gas being delivered to the patient, control the gas delivery apparatus to make an adjustment to the FiO2 setting of the gas being delivered to the patient to an updated FiO2 setting according to the determined FiO2 update, and based at least in part on the updated FiO2 setting, determine the updated PEEP value.

An example, according to some embodiments of the disclosure, of a mechanical ventilator apparatus includes: a gas delivery apparatus, having a patient interface, configured to deliver gas to a patient; and a controller, including a processor and a memory, in communication with the gas delivery apparatus, the controller being configured to: control the gas delivery apparatus to deliver the gas to the patient according to a FiO2 setting and a PEEP setting, wherein the FiO2 setting and the PEEP setting are configured to be adjustable, control the delivery of the gas to the patient at a first delivery time according to a first FiO2 value and a first PEEP value, based at least in part on the first FiO2 value, determine an updated PEEP value selected from at least two PEEP values including the first PEEP value associated with a first FiO2 range and a second PEEP value associated with a second FiO2 range, wherein the second FiO2 range overlaps with the first FiO2 range, wherein a change in the PEEP setting is based at least on a FiO2 value for the FiO2 setting of the gas being delivered to the patient, at a second delivery time that is subsequent to the first delivery time, falling outside of the first FiO2 range, and control the gas delivery apparatus to adjust to the PEEP setting according to the updated PEEP value.

Some implementations may include one or more of the following features. The FiO2 setting may have changed from a previous FiO2 value to the first FiO2 value. The updated PEEP value may be determined is based at least in part on the FiO2 setting and the PEEP setting prior to the adjustment to the PEEP setting. The at least two PEEP values may include at least three PEEP values including a third PEEP value associated with a third FiO2 range, wherein the third FiO2 range overlaps with the second FiO2 range, and wherein changing the PEEP setting requires that the FiO2 setting must have changed so as to fall outside of one of the FiO2 ranges associated with one of the at least three PEEP levels.

An example, according to some embodiments of the disclosure, of a method for controlling mechanical ventilation being provided to a patient includes a controller: controlling a gas delivery system of a mechanical ventilator to deliver gas to the patient according to an FiO2 setting and a PEEP setting, wherein the FiO2 setting and the PEEP setting are adjustable; determining that a current PEEP setting corresponds with a first PEEP value, the first PEEP value being associated with a first FiO2 range; determining a current FiO2 setting of the gas being delivered to the patient; if the current FiO2 setting falls within the first FiO2 range, maintaining the current PEEP setting at the first PEEP value; and if the current FiO2 setting falls outside of the first FiO2 range, adjusting the current PEEP setting from the first PEEP value to a second PEEP value, the second PEEP value being associated with a second FiO2 range that overlaps with the first FiO2 range.

Some implementations may include one or more of the following features. Some embodiments may include the controller, subsequent to the adjustment of the current PEEP setting from the first PEEP value to the second PEEP value, determining an updated FiO2 setting for the gas being delivered to the patient; if the updated FiO2 setting falls within the second FiO2 range, maintaining the current PEEP setting so as to remain at the second PEEP value; and if the updated FiO2 setting value falls outside of the second FiO2 range, adjusting the current PEEP setting from the second PEEP value to the first PEEP value or to a third PEEP value, the third PEEP value being associated with a third FiO2 range.

An example, according to some embodiments of the disclosure, of a mechanical ventilator apparatus includes: a gas delivery apparatus, having a patient interface, for delivering gas to a patient; and a controller, including a processor and a memory, in communication with the gas delivery apparatus, the controller being configured to: control the gas delivery apparatus to deliver the gas to the patient according to a FiO2 setting and a PEEP setting, wherein the FiO2 setting and the PEEP setting are configured to be adjustable, determine that a current PEEP setting corresponds with a first PEEP value, the first PEEP value being associated with a first FiO2 range, determine a current FiO2 setting of the gas being delivered to the patient, based on whether the current FiO2 setting falls within the first FiO2 range, maintain the current PEEP setting at the first PEEP value, based on whether the current FiO2 setting falls outside of the first FiO2 range, adjust the current PEEP setting from the first PEEP value to a second PEEP value, the second PEEP value being associated with a second FiO2 range that overlaps with the first FiO2 range, subsequent to the adjustment of the current PEEP setting from the first PEEP value to the second PEEP value, determine an updated FiO2 setting for the gas being delivered to the patient, based on whether the updated FiO2 setting falls within the second FiO2 range, maintain the current PEEP setting so as to remain at the second PEEP value, and based on whether the updated FiO2 setting value falls outside of the second FiO2 range, adjust the current PEEP setting from the second PEEP value to the first PEEP value or to a third PEEP value, the third PEEP value being associated with a third FiO2 range.

Some implementations may include one or more of the following features. In some embodiments, the third FiO2 range overlaps with the second FiO2 range; wherein a portion of the third FiO2 range is higher than the second FiO2 range, and a portion of the second FiO2 range is higher than the first FiO2 range; and wherein the third PEEP value is higher than the second PEEP value, and the second PEEP value is higher than the first PEEP value.

In some embodiments, if the updated FiO2 setting falls below the second FiO2 range, decrease the current PEEP setting from the second PEEP value to the first PEEP value; and if the updated FiO2 setting falls above the second FiO2 range, increase the current PEEP setting from the second PEEP value to the third PEEP value.

An example, according to some embodiments of the disclosure, of a mechanical ventilator apparatus includes: a gas delivery apparatus, having a patient interface, configured to deliver gas to a patient; an oximetry sensor configured to generate signals representative of an oxygen concentration of the patient's blood; and a controller, including a processor and a memory, in communication with the gas delivery apparatus and the oximetry sensor, the controller being configured to: control the gas delivery apparatus to deliver the gas to the patient according to a FiO2 setting and a PEEP setting, wherein the FiO2 setting and the PEEP setting are configured to be adjustable, receive the signals representative of the oxygen concentration of the patient's blood from the oximetry sensor during the delivery of the gas to the patient, determine the oxygen concentration of the patient's blood based at least in part on the received signals, determine whether at least one of a plurality of PEEP change eligibility conditions is met, and if the at least one of the plurality of PEEP change eligibility conditions is met, control the gas delivery apparatus to adjust the PEEP setting to an updated PEEP value.

Some implementations may include one or more of the following features. In some embodiments, determining that at least one of the plurality of PEEP change eligibility conditions are met includes: determining that the FiO2 setting has not changed by at least a first amount in at least a first period of time, or determining that the oxygen concentration of the patient's blood has been below a desaturation threshold for more than a second period of time. In some embodiments, the controller is configured to determine the at least one of the plurality of PEEP change eligibility conditions is met at a current delivery time.

In some embodiments, determining that at least one of the plurality of PEEP change eligibility conditions are met includes: determining that determining that the FiO2 setting has not changed by at least a first amount in at least a first period of time, or determining that the FiO2 setting has continuously increased for at least a second period of time.

In some embodiments, determining that at least one of the plurality of PEEP change eligibility conditions are met includes: determining that determining that the FiO2 setting has not changed by at least a first amount in at least a first period of time, or determining that the FiO2 setting has continuously decreased for at least a second period of time.

In some embodiments, the updated PEEP value is determined based at least in part on the FiO2 setting and a PEEP value prior to the adjustment to the PEEP setting to the updated PEEP value.

An example, according to some embodiments of the disclosure, of a mechanical ventilator apparatus includes: a gas delivery apparatus, having a patient interface, configured to deliver gas to a patient; an oximetry sensor configured to generate signals representative of an oxygen concentration of the patient's blood; and a controller, including a processor and a memory, in communication with the gas delivery apparatus and the oximetry sensor, the controller being configured to: control the gas delivery apparatus to deliver the gas to the patient according to a FiO2 setting and a PEEP setting, wherein the FiO2 setting and the PEEP setting are configured to be adjustable, receive the generated signals representative of the oxygen concentration of the patient's blood, determine the oxygen concentration of the patient's blood based on the received signals, determine whether a set of PEEP change eligibility conditions is met, the set of conditions including that: if a determined PEEP update includes an increase in PEEP, the PEEP setting has not changed in a first period of time, and if the determined PEEP update includes a decrease in PEEP, the PEEP setting has not changed in a second period of time, the second period of time being different than the first period of time, and if the set of conditions are met, then control the gas delivery apparatus to adjust the PEEP setting according to the determined PEEP update.

An example, according to some embodiments of the disclosure, of a mechanical ventilator apparatus includes: a gas delivery apparatus configured to deliver gas to a patient; an oximetry sensor configured to generate signals representative of an oxygen concentration of the patient's blood; and a controller, including a processor and a memory, in communication with the gas delivery apparatus and the oximetry sensor, the controller being configured to: control the gas delivery apparatus to deliver the gas to the patient according to a FiO2 setting and a PEEP setting, wherein the FiO2 setting and the PEEP setting are configured to be adjustable, receive the generated signals representative of the oxygen concentration of the patient's blood, determine the oxygen concentration of the patient's blood based on the received signals, determine whether a set of PEEP change eligibility conditions are met, the set of conditions including that: if a determined PEEP update includes an increase in PEEP, one or more measures of a hemodynamic status of the patient indicate that the hemodynamic status of the patient is of at least a predetermined level, and if the set of conditions are met, then control the gas delivery apparatus to adjust the PEEP setting according to the determined PEEP update.

An example, according to some embodiments of the disclosure, of a mechanical ventilator apparatus includes a gas delivery apparatus, having a patient interface, configured to deliver gas to a patient; an oximetry sensor configured to generate first signals representative of an oxygen concentration of the patient's blood; a controller, comprising a processor and a memory, in communication with the gas delivery apparatus and the oximetry sensor, the controller being configured to: receive the signals representative of the oxygen concentration of the patient's blood from the oximetry sensor during the delivery of the gas to the patient, determine the oxygen concentration of the patient's blood based at least in part on the received signals, control the gas delivery apparatus to deliver the gas to the patient according to a PEEP setting and an FiO2 setting, wherein: the PEEP setting is configured to be adjustable based at least in part on the FiO2 setting; and the FiO2 setting is configured to be adjustable based at least in part on the determined oxygen concentration of the patient's blood, wherein adjusting the FiO2 comprises: determining a decrease in the oxygen concentration of the patient's blood; determining a correction value, wherein the correction value is increased based at least in part on the determined decrease in the oxygen concentration of the patient's blood; and adjusting the FiO2 setting by adding the correction value to the FiO2 setting.

Some implementations may include one or more of the following features. In some embodiments, the controller is configured to determine the decrease in the oxygen concentration of the patient's blood from a previous time or time period to a current time or time period, wherein the previous time or time period is immediately previous to the current time or time period. Determining the correction value may include determining an increase in the correction value calculated based on a constant term multiplied by a term representing the determined decrease in the oxygen concentration of the patient's blood. Furthermore, determining the correction value may include determining an increase in the correction value calculated based on a constant term multiplied by a term representing the determined decrease in the oxygen concentration of the patient's blood, wherein the constant term has a magnitude of, for example, between 0 and 0.1, between 0.1 and 0.2, between 0.2 and 0.3, or within another suitable range.

In some embodiments, the correction value may not be decreased based at least in part on a determined increase in the SpO2. The decrease in the oxygen concentration of the patient's blood may be determined based at least in part on a moving average of measured SpO2 values over the previous time period relative to a moving average of measured SpO2 values over the current time period.

Adjusting the FiO2 setting may include creating a tendency for the FiO2 to change so as to cause the SpO2 to approach a target SpO2, and may include using a term calculated as a constant multiplied by a value representing a difference between a measure of recent SpO2 and the target SpO2. The tendency may be greater if the measure of recent SpO2 is outside of an SpO2 range that includes the target SpO2. The range may be 0.93-0.99. Adjusting the FiO2 setting may include, if the measure of recent SpO2 is outside of the SpO2 range, using a term calculated as a first constant multiplied by the difference between the measure of recent SpO2 and the target SpO2, and, if the measure of recent SpO2 is inside of the SpO2 range, using a term calculated as a second constant multiplied by the difference between the measure of recent SpO2 and the target SpO2, wherein the second constant is less than the first constant.

An example, according to some embodiments of the disclosure, of a mechanical ventilator apparatus, includes: a gas delivery apparatus, having a patient interface, configured to deliver gas to a patient; an oximetry sensor configured to generate first signals representative of an oxygen concentration of the patient's blood; a capnography sensor configured to generate second signals representative of a carbon dioxide concentration or partial pressure of expired gas from the patient; a controller, comprising a processor and a memory, in communication with the gas delivery apparatus and the oximetry sensor, the controller being configured to: receive the first and second signals; determine the oxygen concentration of the patient's blood based at least in part on the received first signals and the carbon dioxide concentration or partial pressure of the expired gas of the patient based at least in part on the second signals; and control the gas delivery apparatus to deliver the gas to the patient according to a FiO2 setting, a PEEP setting, and a Ve setting, wherein: the FiO2 setting is configured to be adjustable based at least in part on the determined oxygen concentration of the patient's blood; the PEEP setting is configured to be adjustable based at least in part on the FiO2 setting; and the Ve setting is configured to be adjustable based at least in part on the determined carbon dioxide concentration or partial pressure of the expired gas of the patient.

The Ve setting may be configured to be increased or decreased based at least in part on the carbon dioxide concentration or partial pressure of the expired gas of the patient. Determining the carbon dioxide concentration or partial pressure may include using EtCO2 or an EtCO2 average over a period of time. The period of time may be one minute. The Ve setting may be configured to be increased based at least in part on the carbon dioxide concentration or partial pressure of the expired gas of the patient being above a first threshold, and an updated Ve setting may be calculated as Ve*1.1.

The Ve setting may be configured to be decreased based at least in part on the carbon dioxide concentration or partial pressure of the expired gas of the patient being below a second threshold, wherein the second threshold is lower than the first threshold, and an updated Ve setting may be calculated as Ve/1.1.

The first threshold may be between 50-60 mm Hg or 55 mm Hg. The second threshold may be between 25-35 mm Hg or 30 mm Hg.

A PIP setting may be configured such that, if the carbon dioxide concentration or partial pressure of the expired gas of the patient is between the first threshold and the second threshold, then the PIP setting is updated based at least in part on a Vt setting.

### BRIEF DESCRIPTION OF THE DRAWINGS

Various aspects of embodiments of the present disclosure are discussed below with reference to the accompanying figures, which are not intended to be drawn to scale. The figures are included for illustrative purposes and a further understanding of the various aspects and examples, and are incorporated in and constitute a part of this specification, but are not intended to limit the scope of the disclosure. The drawings, together with the remainder of the specification, serve to explain principles and operations of the described and claimed aspects and examples. In the figures, identical or nearly identical components that are illustrated in various figures may be represented by like numerals. For purposes of clarity, not every component may be labeled in every figure. Herein, where a single element is recited, it is implied that one or more may be included in various embodiments.

FIG. 1 illustrates an example system incorporating closed loop control in mechanical ventilation of a patient, in accordance with embodiments of the present disclosure.

FIG. 2 illustrates an example portable ventilation system incorporating closed loop control, in accordance with embodiments of the present disclosure.

FIG. 3 is a block diagram illustrating example software-architecture related aspects of a ventilation system incorporating closed loop control, in accordance with embodiments of the present disclosure.

FIG. 4A is a flow diagram illustrating an example method of operation of ventilation control aspects of a ventilation system incorporating closed loop control, in accordance with embodiments of the present disclosure.

FIG. 4B is a flow diagram illustrating an example method of operation of ventilation control aspects of a ventilation system incorporating closed loop control, in accordance with embodiments of the present disclosure

FIG. 5 is a flow diagram illustrating an example method of operation of an FiO2 control aspects of a ventilation system incorporating closed loop control, in accordance with embodiments of the present disclosure.

FIGS. 6, 7A and 7B are flow diagrams illustrating example methods of operation of PEEP control aspects of a ventilation system incorporating closed loop control, in accordance with embodiments of the present disclosure.

FIG. 8 is a block diagram illustrating aspects of example PEEP selection rules, in accordance with embodiments of the present disclosure.

FIG. 9 is a flow diagram illustrating an example multi-mode method of operation of a ventilation system incorporating closed loop control, in accordance with embodiments of the present disclosure.

FIG. 10 is a flow diagram illustrating an example method of operation of an initiation mode of a ventilation system incorporating closed loop control, in accordance with embodiments of the present disclosure.

FIG. 11 is a flow diagram illustrating an example method of operation of an example test breaths mode of a ventilation system incorporating closed loop control, in accordance with embodiments of the present disclosure.

FIG. 12A is a flow diagram illustrating an example method of operation of a gain related algorithm, in accordance with embodiments of the present disclosure.

FIGS. 12B-12E are graphical illustrations of example animal study results tracking FiO2 setting relative to SpO2, in accordance with embodiments of the present disclosure.

FIG. 13 is a flow diagram illustrating aspects of an example method of operation of a desaturation related algorithm, in accordance with embodiments of the present disclosure.

FIG. 14 is a flow diagram illustrating aspects of an example method for determination of ventilation parameters based on calculated respiratory parameters, in accordance with embodiments of the present disclosure.

FIG. 15 illustrates an example respiratory pressure waveform and related model fitted waveform that can be used in calculation of ventilation parameters, in accordance with embodiments of the present disclosure.

FIG. 16 illustrates an example respiratory flow waveform that can be used in calculation of ventilation parameters, in accordance with embodiments of the present disclosure.

FIG. 17 illustrates an example respiratory volume waveform that can be used in calculation of ventilation parameters, in accordance with embodiments of the present disclosure.

FIG. 18 illustrates example animal study results demonstrating aspects of closed loop control, including FiO2 setting adjustment based on measured SpO2, and PEEP setting adjustment based on FiO2 setting, in accordance with embodiments of the present disclosure.

FIGS. 19A-19B illustrate example animal study results demonstrating aspects of closed loop control, including settings adjustment based on EtCO2, in accordance with embodiments of the present disclosure.

FIG. 20A illustrates a simplified example portable ventilator and display, with FiO2 closed loop control enabled, in accordance with embodiments of the present disclosure.

FIG. 20B illustrates a simplified example portable ventilator and display, with FiO2 closed loop control paused, in accordance with embodiments of the present disclosure.

FIG. 20C illustrates a simplified example portable ventilator and display, with PEEP closed loop control enabled, in accordance with embodiments of the present disclosure.

FIG. 20D illustrates a simplified example portable ventilator and display, with PEEP closed loop control paused, in accordance with embodiments of the present disclosure.

FIG. 20E illustrates a simplified example portable ventilator and display, with FiO2 and PEEP closed loop control enabled, in accordance with embodiments of the present disclosure.

FIG. 20F illustrates a simplified example portable ventilator and display, with FiO2 and PEEP closed loop control paused, in accordance with embodiments of the present disclosure.

FIG. 20G illustrates a simplified example displayed menu of a portable ventilator display, in accordance with embodiments of the present disclosure.

FIG. 20H illustrates a simplified example displayed message of a portable ventilator display, with FiO2 CLC enabled, in accordance with embodiments of the present disclosure.

FIG. 20I illustrates a simplified example displayed message of a portable ventilator display, with PEEP CLC enabled, in accordance with embodiments of the present disclosure.

FIG. 21 illustrates aspects of an example pneumatic system that can be used with a portable ventilator, in accordance with embodiments of the present disclosure.

FIG. 22 illustrates aspects of example external gas supply systems that can be used with a portable ventilator, in accordance with embodiments of the present disclosure.

FIG. 23 illustrates aspects of example patient circuits that can be used with a portable ventilator, in accordance with embodiments of the present disclosure.

### DETAILED DESCRIPTION

Herein, in some instances, variations of a term may be utilized that may refer to the same or similar concepts, and certain terms may have meanings that are informed by a particular context. Various ventilation parameter related terms or abbreviations, including fraction of inspired oxygen (FiO2), positive end-expiratory pressure (PEEP) and others, may refer to ventilation related settings, even though the word “setting” may or may not be stated. Furthermore, reference to a ventilation parameter or parameter setting may be used to refer to the parameter in a conceptual or definitional sense, or the value associated with a particular setting (e.g., “PEEP of 5 cm H2O”). In the ventilation field, a PEEP setting may sometimes be called a Baseline Airway Pressure (BAP) setting, with both terms referring to the same setting. As such, it should be noted that, herein, with reference to a setting, PEEP and BAP are to be understood to be used alternatively and to refer to the same setting. A user, as described herein, may include an individual operating, supervising or in whole or in part responsible for operation of a device such as a portable ventilator, even if, during a particular period of time while the device is operating, the user may not be interacting with the device.

An alert or alarm, as used herein, may be presented for the attention of a user, such as by being visually or audibly presented, such as via a display, graphical user interface (GUI) or speaker of a device. However, an alert or alarm may also include reference to alert or alarm conditions that are algorithmically identified, recognized or determined by a computerized device and not necessarily presented or displayed. Herein, the term optimizing may include, for example, improvement or improved operation in one or more aspects, for example, relative to an actual, potential or hypothetical less optimized situation or less optimized operation.

Herein, a ventilator can include, for example, a ventilator or ventilation device, apparatus or system, whether or not portable, and whether or not functionality other than ventilation aspects is provided by the device, apparatus or system. Herein, the term adjusting can include changing as well as not changing or maintaining without change, as may be appropriate. Herein, a determined parameter value can include a determined estimated or determined approximated value for the parameter. Herein, the term monitoring can refer to or include, for example, monitoring or tracking performed by a computerized device utilizing one or more algorithms and not by a person or user, or monitoring by a person or user, or both. Herein, the term continuous can include, among other things, on a periodic basis (with identical or different periods), on a frequent basis, on a repeated basis, or cyclically, for example.

Herein, terms such as hypercapnia, hypocapnia and normocapnia are not intended to be limited to particular ranges or clinical meanings, but are used in a relative sense, such as to indicate relatively high, relatively low, or intermediate, in a particular embodiment, for example. Furthermore, in some embodiments, associated ranges, or some of them, may overlap. In some embodiments, normocapnia may have a lower EtCO2 threshold of, e.g., in mm Hg, 20, 25, 30, 35, 40, 45 or 50 and an upper threshold of, e.g., in mm Hg, 30, 35, 40, 45, 50, 55 or 60. Hypercapnia may include an EtCO2 of at or above a threshold of, e.g., in mm Hg, 30, 35, 40, 45, 50, 55 or 60. Hypocapnia may include an EtCO2 of at or below a threshold of, e.g., in mm Hg, 20, 25, 30, 35, 40, 45 or 50.

The term closed loop control, as used herein, may refer to control of one or more ventilation related or patient related parameters, such as with relatively little or no required user action, participation or intervention, and can include reference to, but is not limited to reference to, fully automated or fully automatically regulated control. Closed loop control may include, for example, device facilitated or algorithmically facilitated tracking, control and adjustment of one or more parameters, which may or may not include user involvement or participation. Where user involvement or participation is included, it may include, for example, confirming a suggested or recommended ventilation setting change or configuration, deciding on implementing a course of action, selecting one of several suggested courses of action, responding to a presented alert or alarm, or other decisions, choices or actions. User involvement or participation could also include, for example, setting or changing a parameter, where a closed loop control algorithm proceeds from there, initially according to the user-set or user-changed parameter setting. In various embodiments, if there is user involvement, it may be, for example, among other things, in whole or in part user-initiated, or in whole or in part prompted, suggested, recommended or required.

In some embodiments, closed loop control may be utilized but may be subject to manual adjustment or override by the user. For example, in some embodiments, although FiO2 and PEEP (BAP) may be algorithmically and automatically controlled, a user may be able to intervene and manually change the FiO2 and/or PEEP (BAP) setting. In some embodiments, following any manual adjustments, closed loop control of FiO2 and/or PEEP (BAP) may resume from that point, at least until any further manual adjustments are made.

Various embodiments as described herein may apply to, for example, out-of-hospital or pre-hospital patient care (although not limited to such care). Some aspects of embodiments described herein take into account practical factors relating to such care contexts. For example, in pre-hospital patient care, the care provider may be less trained than a hospital-based care provider. Furthermore, no support or less support from other care providers and/or hospital systems may be available. Also, pre-hospital care may involve less patient and equipment physical stability, and may involve movement or transport. Additionally, pre-hospital patient care may extend for long periods of time, sometimes extending to many hours, a day or several days. During this time, the resulting physical and mental stress, and overall exhaustion, of the patient and the care provider can be significant factors to consider in determining optimal ventilator operation, procedures and algorithms. As such, in some embodiments, these pre-hospital conditions are taken into account, such as in connection with determination of parameter values, ranges and thresholds, frequency of change of parameters, overall simplicity, and balances between parameter stability and the need for adjustment. In some embodiments, such balances and optimization approaches may be thought of as providing or favoring a form of conceptual “guardrails” in view of the particular concerns and elevated potential risks that tend to accompany pre-hospital care contexts and situations.

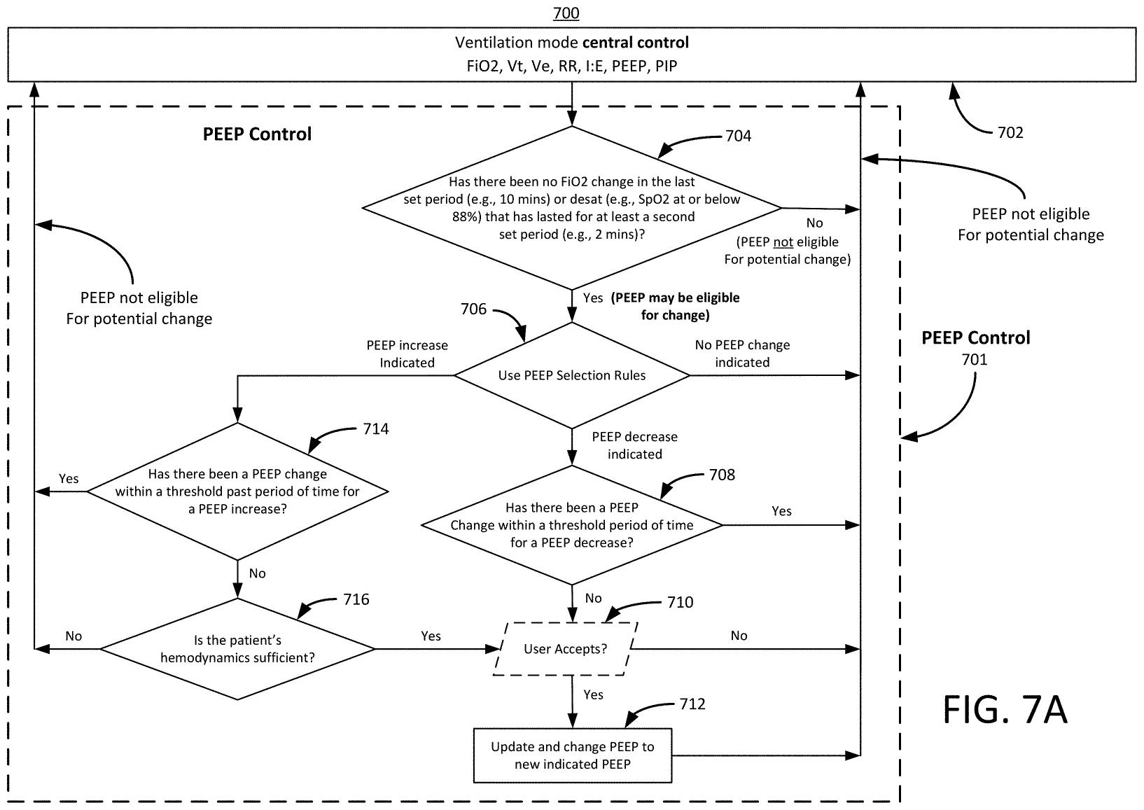

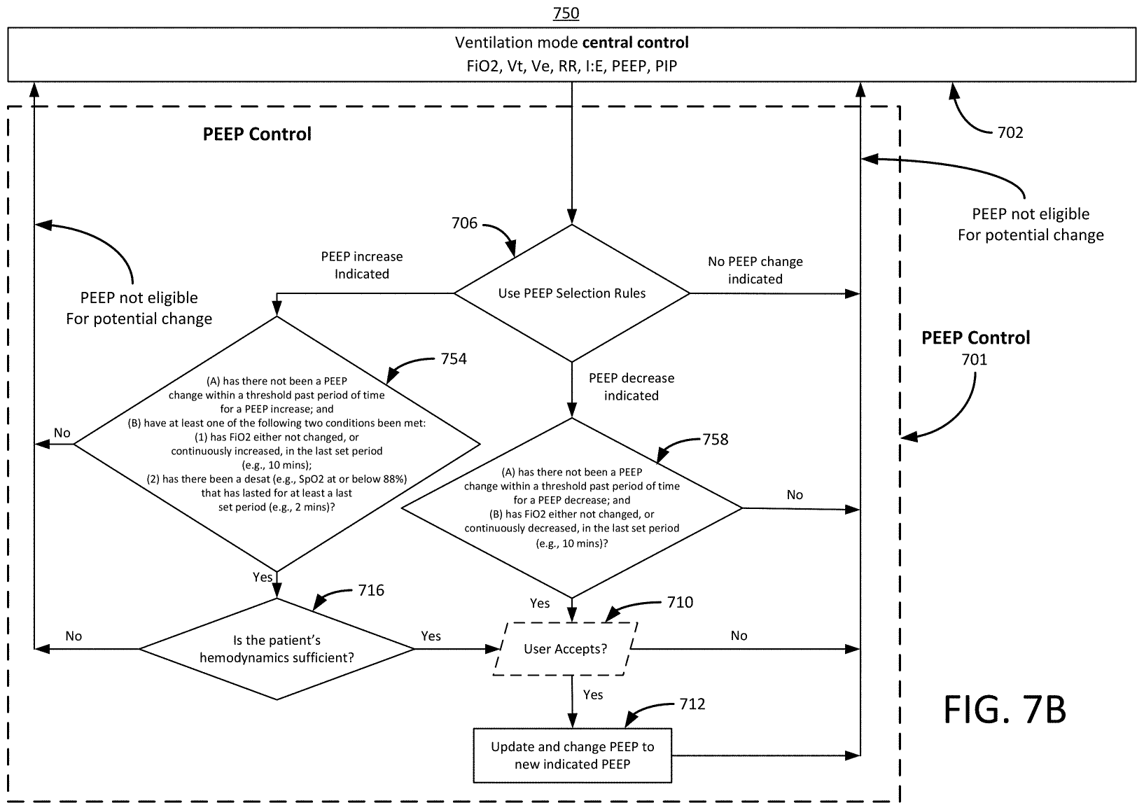

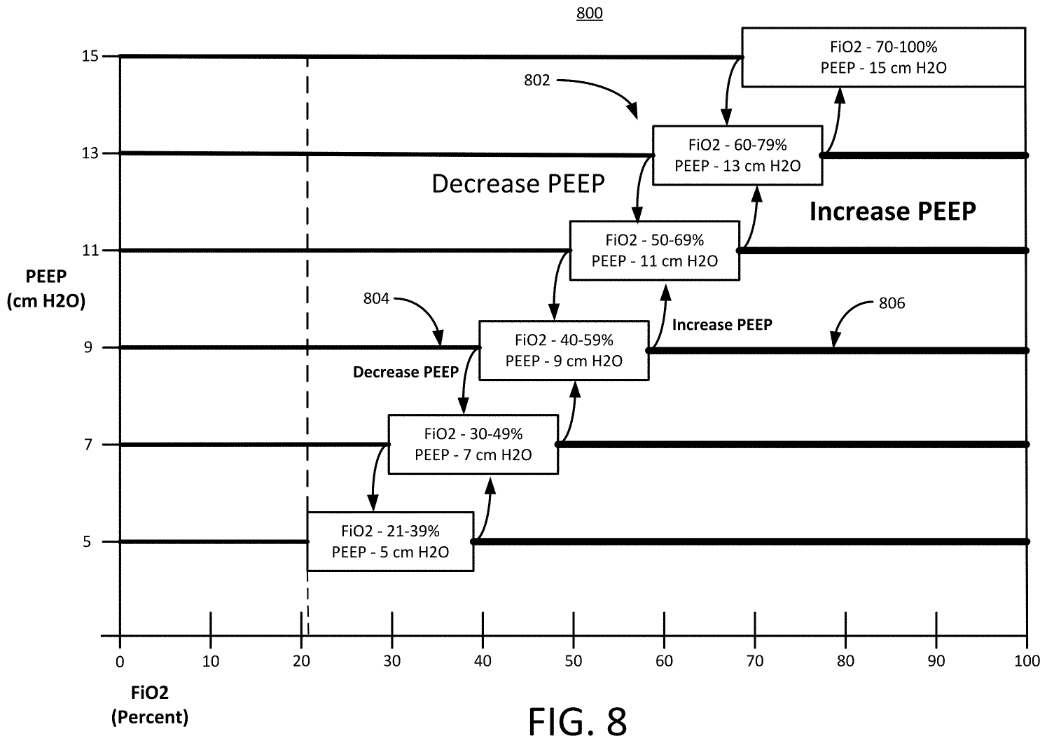

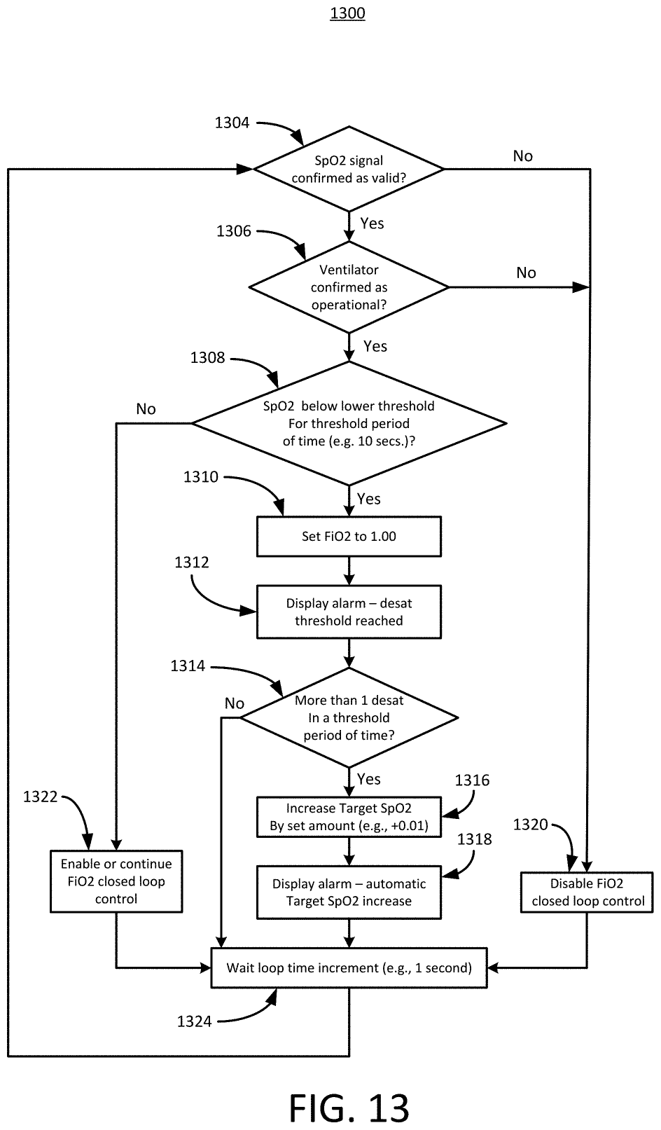

In some embodiments, overall, optimization in view of pre-hospital care related factors can favor increased simplicity, increased stability and a generally more limited or conservative approach with regard to ventilation parameter value determinations and adjustments. Such approaches may include one or several of the following. Some embodiments may include frequent user checks and confirmations, user warnings, or user alerts. Some embodiments may include determined and displayed context-sensitive guidance provided to the user, which may provide additional user support, given pre-hospital circumstances, in which a user with limited training may be providing care under stressful and distracting circumstances. For example, some embodiments provide optimized approaches including greater overall stability, less frequent changes in parameters, and more or greater change in conditions required for changes in parameter values. Furthermore, some embodiments include use of more conservative parameter values, or more conservative high or low limits, such as with regard to parameters that may present a potentially high degree of patient risk. These may include, for example, PEEP, PIP, driving or plateau pressure, Vt and Ve. For example, aspects that may reflect such optimization may include PEEP change eligibility rules, as described with reference to FIG. 7, which can limit and specify conditions required for PEEP changes or potential PEEP changes, and/or can be used in determining a maximum PEEP. Aspects reflecting such optimization may further include PEEP selection rules, as described with reference to FIG. 8, which can specify a change in FiO2 required to indicate a PEEP change in view of a current PEEP level. However, in some circumstances, pre-hospital considerations may warrant optimizations or balances in favor of rapid or increased changes, such as, for example, increasing FiO2 to 100% upon detection of a defined patient desaturation condition, as described with reference to FIG. 13.

In various embodiments, FiO2 may be continuously adjusted based on measured patient SpO2, which is used as an indicator of patient oxygen saturation. Generally, a lower SpO2, or decreasing SpO2 (as may be determined, for example, based on a single or current SpO2 measurement, or several SpO2 measurements over a recent period of time) may tend to indicate a “sicker” patient, or patient with more impaired lung or respiratory system function, who is in need of more oxygenation support. In some embodiments, generally, when a patient's SpO2 is below a target level (such as a pre-determined SpO2 value), and potentially also based at least in part on how much below, and/or decreasing, FiO2 may algorithmically tend to be increased in order to increase support of a patient's oxygenation. When SpO2 is above the target level, and potentially also based at least in part on how much above, FiO2 may algorithmically tend to be decreased, since the patient may not be in need of as much oxygenation support. As such, FiO2 may be increased or decreased based at least in part on the need of the patient as indicated at least in part by SpO2 and/or, for example, some other indication(s), such as one or more other non-invasively sensed, measured or determined indications of patient oxygen saturation. Algorithmically determined factors, such as derivative gain and proportional gain factors, may affect a direction (increase or decrease) and amount of FiO2 adjustment.

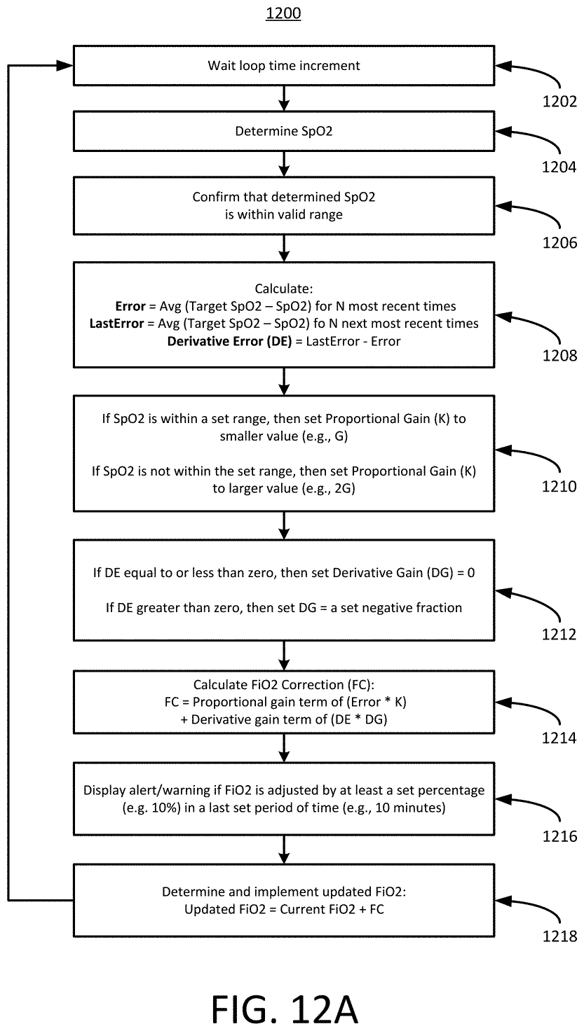

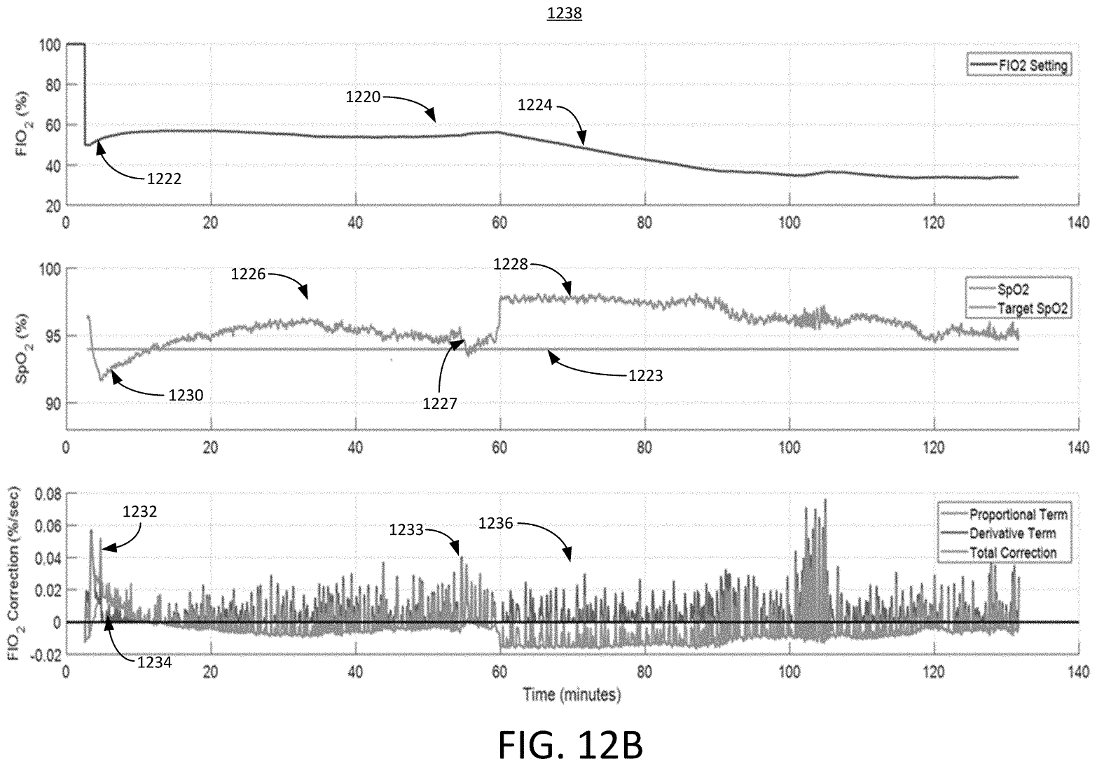

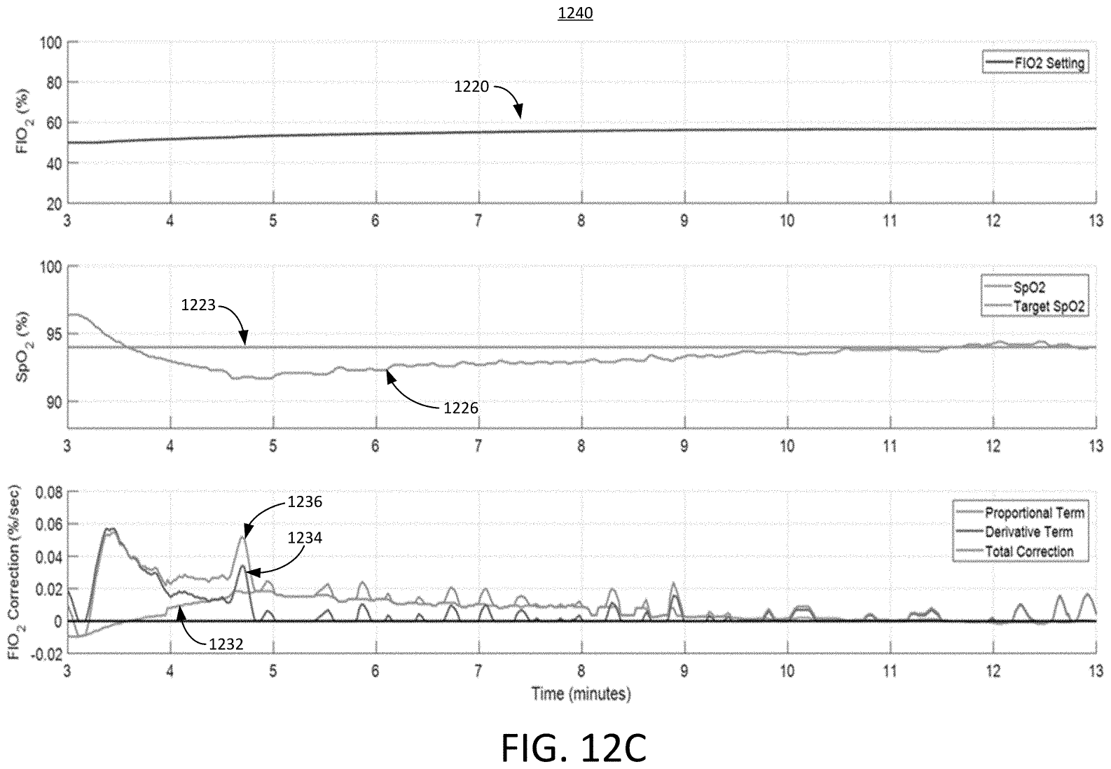

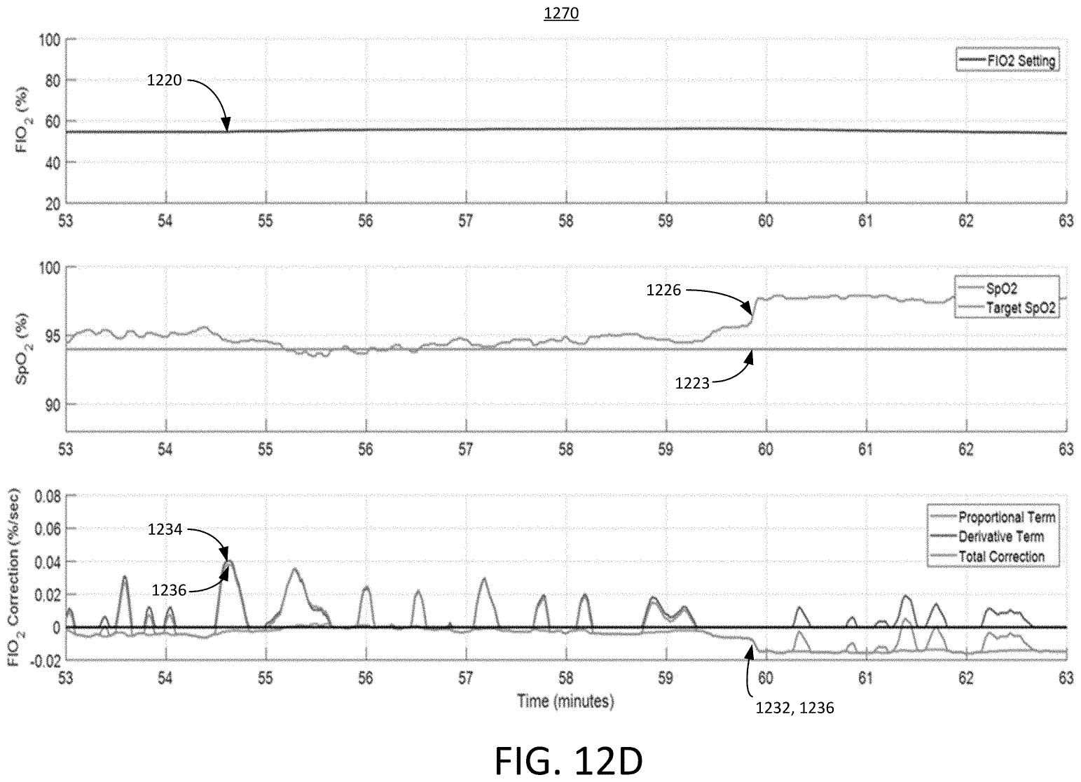

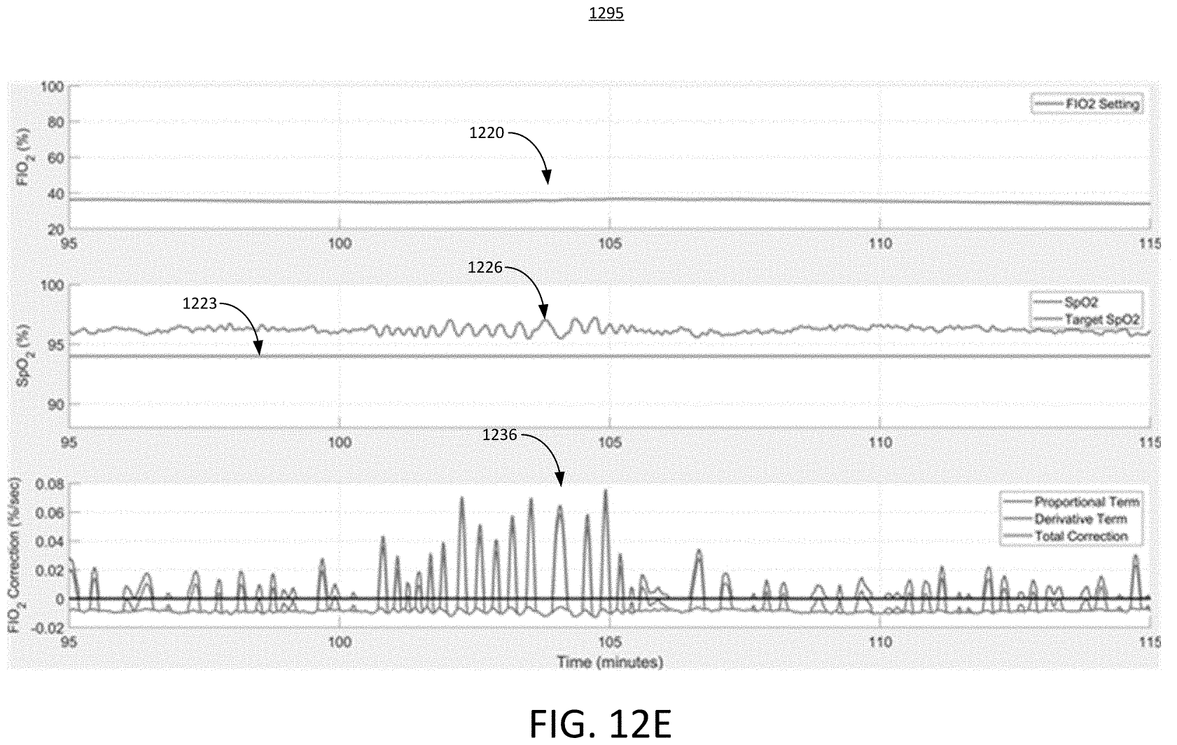

In some embodiments, one or more previously measured SpO2 values may be taken into account algorithmically in determining an FiO2 adjustment. In particular, in some embodiments, proportional gain and derivative gain may take into account one or more previously measured SpO2 values. Generally, in some embodiments, derivative gain may take into account an effective rate of decrease in SpO2 for some period of time up to and including the time of the currently measured SpO2 (such as may include use of a moving average). Derivative gain may tend to increase the FiO2 adjustment when SpO2 is decreasing, such as in a manner that may be associated with, or proportional to, a determined rate of SpO2 decrease. However, in some embodiments, derivative gain may not operate to tend to decrease FiO2 when SpO2 is increasing, and may in this regard be asymmetric in operation. Furthermore, in some embodiments, proportional gain may take into account a determined magnitude of the difference between the current (or current and recent) SpO2 and a target SpO2, where a larger difference may lead to a greater adjustment. Embodiments of algorithms for closed loop control of FiO2, including use of derivative gain and proportional gain, are described with reference to FIG. 12A.

In some embodiments, PEEP is adjusted based at least in part on FiO2, but also based at least in part on the current PEEP. Since FiO2 may be adjusted based at least part on patient SpO2, and since SpO2 may be associated with how “sick,” compromised or functionally impaired the patient's lungs or respiratory system are, conceptually, FiO2 may to some degree represent or effectively operate as a surrogate or indicator of how “sick” the patient's lungs or respiratory system are. For example, in some instances, a relatively low and/or decreasing SpO2 may indicate substantial and/or increasing degree of functional lung impairment, and may result in a relatively high FiO2. This high FiO2 may, under appropriate circumstances, warrant and lead to an increase in PEEP (as may be determined with reference to, for example, PEEP change eligibility rules and PEEP selection rules), where PEEP may help essentially open alveoli and support respiratory function. Conversely, in some instances, a relatively high and/or increasing SpO2 may indicate less substantial and/or decreasing lung impairment (i.e., generally, the patient may be breathing “better” on their own), and may lead to a relatively low FiO2, which may, in some instances, warrant and lead to a decrease in PEEP (as may be determined with reference to, for example, PEEP change eligibility rules and PEEP selection rules).

As such, adjusting PEEP based at least in part on FiO2 may result in PEEP being adjusted based at least in part on an indication of how “sick” or functionally impaired the patient's lungs or respiratory system may be.

PEEP can support a patient by, for example, increasing alveolar pressure and volume, which can essentially distend and prevent the collapse of alveoli, improving oxygenation. However, too high a PEEP can cause risk to a patient. For example, too high a PEEP can lead to an increased thoracic pressure, which in turn can lead to a decrease in patient blood pressure by inhibiting or otherwise limiting venous blood return, causing low blood pressure related risk to the patient. Also, since it can take some time for an increased PEEP to have full effect on alveoli, the full effect of an increased PEEP may take some time, such as minutes, to fully emerge. That can be a reason to avoid increasing PEEP too rapidly, since there may be a possibility of essentially “overshooting the mark” in terms of PEEP increase rapidity and creating patient risk. Furthermore, overly high or over-frequent changing of the PEEP can create other risks to the patient, such as risk of barotrauma, damaging or rupturing alveoli, pneumothorax, pulmonary interstitial emphysema, pneumomediastimum, fibrogenesis, inflammation or a damaging immune response. As such, while it can be important to increase PEEP where warranted, in can also be important to avoid increasing PEEP too much or too frequently. In some embodiments, algorithms including PEEP change eligibility rules and PEEP selection rules provide an optimized approach to PEEP control, balancing assessed need for PEEP adjustment with need for avoiding too high a PEEP and avoiding over-frequent changes in PEEP. A more conservative or slower approach to decreasing PEEP may be warranted, relative to increasing PEEP, since decreasing PEEP is not undertaken in order to address a need of the patient for increased support.

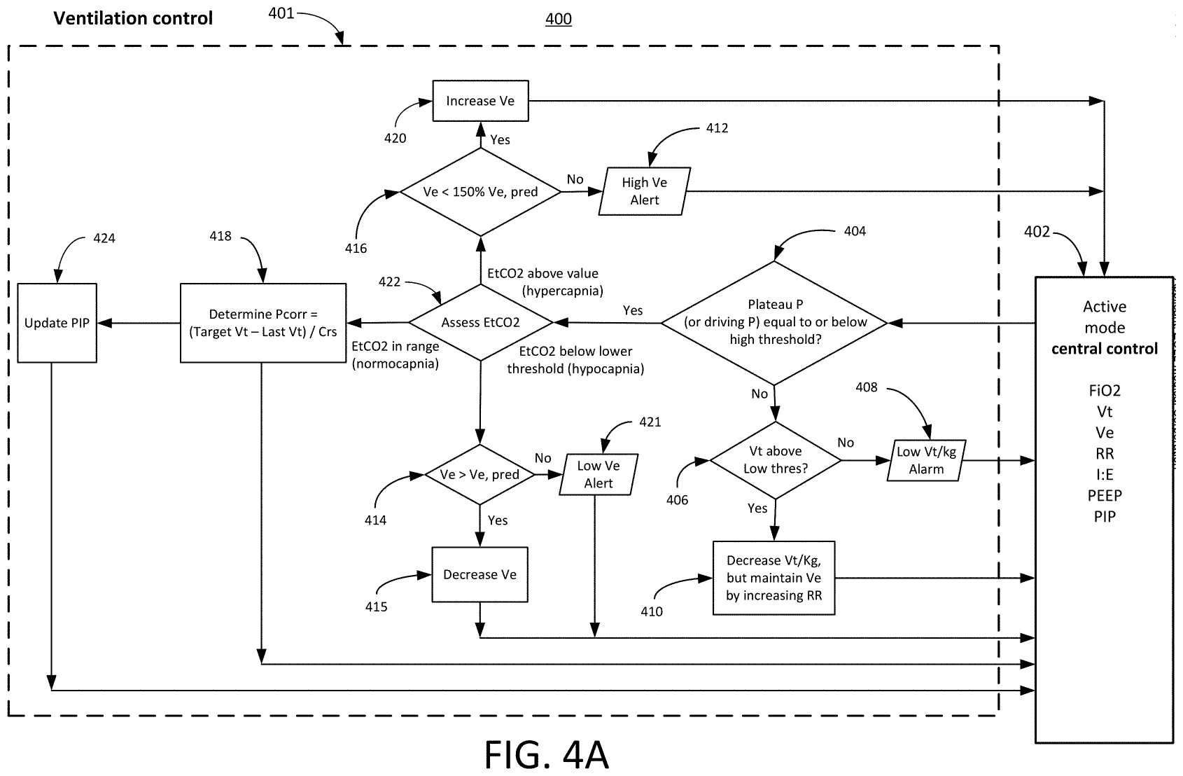

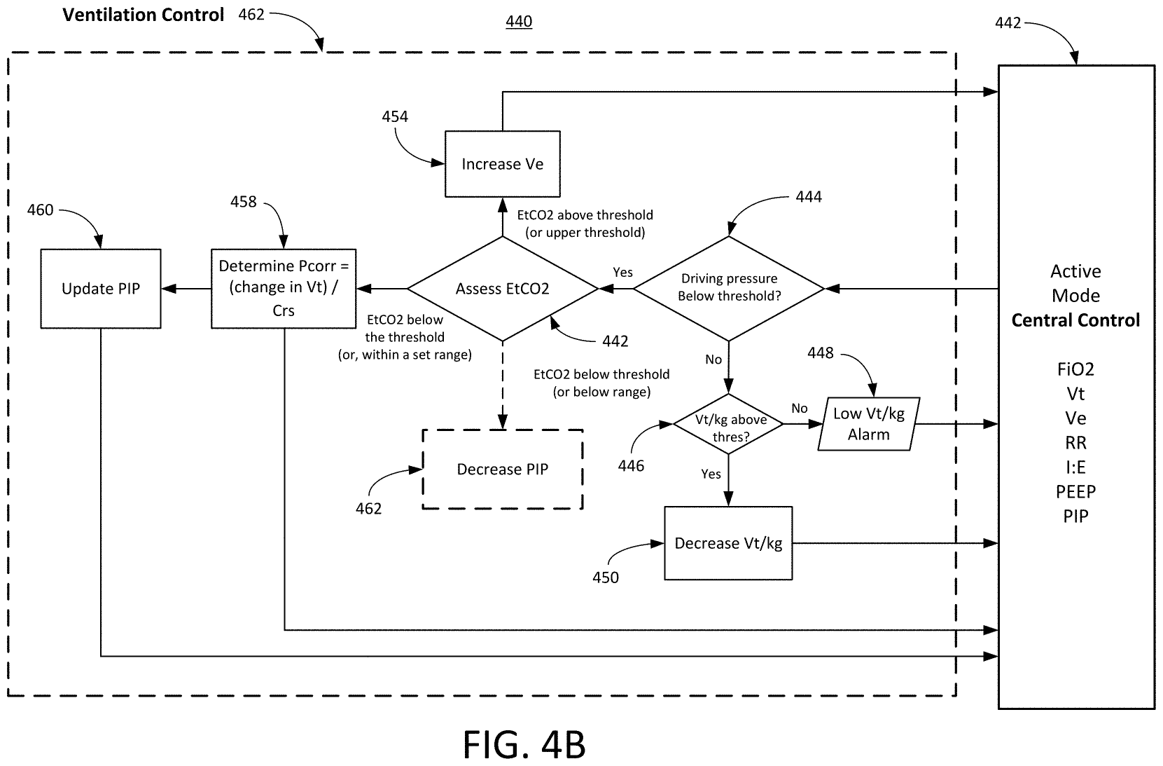

In some embodiments, various parameters in addition to FiO2 and PEEP may be continuously adjusted or adjusted using closed loop control. These may include, for example, adjustments that may be based at least in part on EtCO2, which may indicate or suggest normocapnia, hypercapnia or hypocapnia. For example, in some embodiments, parameters including Vt, Ve and PIP may be continuously adjusted, as described with reference to FIG. 4A.

Apparatus, systems and methods are presented herein that relate to providing mechanical ventilation to a patient, as well as controlling, optimizing, regulating and automating aspects thereof, including with regard, for example, to safety and performance. Systems, apparatus and methods presented herein may include closed loop control of one or more parameters associated with the mechanical ventilation, the patient or both. While embodiments of the present disclosure are applicable to both hospital and non-hospital settings, some embodiments may be particularly advantageous in non-hospital, out-of-hospital or field settings, such as for use with, or as, a portable ventilator or ventilation system to be operated or overseen by a user or care provider with limited ventilation related training or experience. In some embodiments of closed loop control as described herein, by reducing necessary user monitoring or control of various ventilation or patient related parameters, while yet optimizing such parameters, patient care, safety, outcomes and even survival rates can be significantly improved, particularly in pre-hospital situations. In some embodiments, one or more initial ventilation settings may be determined or optimized based at least in part on patient data, which may be at least in part user provided, such as the gender and height, or estimated height, of the patient.

In some embodiments, during ventilation of a patient, an oxygen concentration of the patient's blood, such as SpO2, or another patient oxygenation related parameter, is continuously measured, determined and tracked. Based at least in part on the determined oxygen concentration of the patient's blood, an FiO2 setting of the ventilator, or other oxygen related setting that may be associated with provided breathing gas, is appropriately adjusted to an updated FiO2 setting, such as may include appropriate actuation of an oxygen source valve, actuation, adjustment, opening/increasing the opening of, or closing/increasing the closing of one or more other valves or settings associated with an oxygen source or concentrator, or in other ways. In various embodiments, based at least in part on the updated FiO2 setting, or based at least in part on the adjustment to the FiO2 setting, a PEEP setting of the ventilator is appropriately adjusted, such as may include appropriate actuation of an exhalation valve, actuation or adjustment of one or more other valves or settings, or in other ways.

The monitoring of the oxygen concentration of the patient's blood, checking/updating of the FiO2 setting and checking/updating of the PEEP setting may be performed on a very frequent basis, such as, e.g., every fraction of a second, every second, every more than one second or several seconds, every minute or several minutes, or based on irregularly or varying duration periods. In some embodiments, FiO2 may be adjusted more frequently than PEEP. Monitoring and adjusting of parameters may be implemented or facilitated by one or more controllers that may execute one or more appropriate algorithms stored in one or more memories. Additionally, optimized initial settings may be determined and utilized.

In some embodiments, PEEP selection rules are provided. According to some embodiments, a PEEP setting may be changed only if an FiO2 setting changes so as to fall outside of an FiO2 range associated with the current PEEP setting. Moreover, in some embodiments, each of a number of possible PEEP settings is associated with a particular FiO2 range, but FiO2 ranges, or adjacent FiO2 ranges, may overlap. As a result, the current PEEP setting is maintained unless the FiO2 setting changes not only (a) enough to fall into a portion of the current FiO2 range that overlaps with another FiO2 range associated with a different PEEP setting, but (b) enough to go beyond the overlapping portion as well as into an FiO2 range associated with a different PEEP setting. FIG. 8 and the description thereof illustrate a detailed example of embodiments incorporating these features. Generally, requiring FiO2 change beyond the applicable range, as described in embodiments herein, in order to change the PEEP setting (whether an increase or decrease) can effectively limit the rate or frequency of change of PEEP, balanced with the need to change PEEP over time when warranted. This, in turn, can have advantages such as allowing more time for a previous PEEP change to have full effect (since a PEEP change takes some time to have full effect on alveoli) before changing PEEP. It can also have the advantage of avoiding abrupt or over-frequent PEEP oscillations (which can disrupt, break open, or otherwise damage alveoli) and achieving overall throttling or smoothing of PEEP change over time, for example.

In some embodiments, the above can be viewed as creating an intended, throttled or balanced tendency to maintain, rather than change, the current PEEP setting, while yet indicating PEEP change under appropriate circumstances. In some embodiments, this, in turn, can improve patient safety and outcome, given that, for example, while PEEP setting changes are necessary under appropriate circumstances, overly frequent, sudden and/or large changes in PEEP setting may cause serious risk to the patient, since over-frequent PEEP changes can break apart alveoli and damage a patient's lungs, for example. Therefore, it can be optimal to appropriately balance various factors, which can be accomplished by some embodiments disclosed herein.

Furthermore, in some embodiments, possible or allowed PEEP settings are determined to include a discrete set of particular PEEP settings, or levels, each of which is associated with an FiO2 range, where adjacent ranges may overlap. In some embodiments, a PEEP setting can only be adjusted so as to change at most by one level up or down, even if, for example, the FiO2 setting changes dramatically.

Additionally, some embodiments provide or utilize particular rules or conditions, or PEEP change eligibility rules or conditions (and/or ineligibility rules or conditions), which must be met in order for a PEEP setting change to be allowed and actually made, even if, for example, it may be indicated by the PEEP selection rules. In some embodiments, even if a PEEP setting might otherwise be indicated by the PEEP selection rules, unless and until the specified eligibility conditions are met (or ineligibility conditions avoided), PEEP is be maintained at the current setting. In some embodiments, for example, these conditions or rules provide an appropriate or optimal balance of factors affecting performance and safety, such as by balancing the advantage of appropriate change and rate of change with the advantages of, for example, appropriate relative stability and limited rate of change.

Generally, in some embodiments, the time periods required for PEEP change eligibility may reflect a balance of the advantages of stability relative to the need for change, as described above. In some embodiments, the PEEP change eligibility rules may include that PEEP has not been changed in at least a specified period of time, and the specified period of time may be different depending on whether a PEEP increase or decrease is being evaluated. Furthermore, in some embodiments, the specified period of time for a PEEP increase (e.g., in minutes, between 2-3, 3-4, 4-5, 5-7, 7-10, 10-15, 15-20, 20-25, 25-30, 30-35 or 35-40) may be less than the specified period of time for a PEEP decrease (e.g., in minutes, between 10-20, 20-30, 30-40, 40-50, 50-60, 60-70, 70-80, 80-90, 90-100, 100-110 or 110-120). Reasons for this may include that, if a PEEP increase is indicated, that can mean that the patient's lungs are becoming more “sick” or functionally impaired, which may warrant relatively fast action to change PEEP in order to provide the patient with more support, whereas decreasing PEEP does not increase patient support, and so a longer period of time for a PEEP decrease may be warranted.

Furthermore, in some embodiments, PEEP change eligibility rules may include that the FiO2 setting has not changed, or has not changed by at least a particular amount, during a first period of time (which can be termed, for convenience, a “steady state” situation with regard to FiO2), or the patient has been in what may be termed a desaturation condition for at least a second period of time, such as may be indicated by patient SpO2, as described with reference to FIG. 13. In some such embodiments, PEEP can only be changed, even if a PEEP change is otherwise indicated, if at least one of two conditions are met: (1) the FiO2 has been steady state (no change in FiO2, or no change beyond a certain minimal threshold, e.g., 1%, or, e.g., between 0.1-0.5%, between 0.5%-1%, between 1-1.5%, between 1.5%-2%, between 2-10% or between 10-20%) for the first period of time (e.g., in seconds, 0-15, 15-30, or 30-60, or, in minutes, 1-2, 2-5, 5-10, 10-12, 12-15, 15-20, 30-45 or 45-60), or (2) the patient has been in a desaturation condition (e.g., SpO2 of at or below, e.g., 85%, 86%, 87%, 88%, 89%, 90%, 91% or 92%) for at least a second period of time (e.g., in seconds, 0-15, 15-30, or 30-60, or, in minutes, 1-2, 2-5, 5-10, 10-12, 12-15 or 15-20).

In some embodiments, conditions (1) and (2) may prevent allowing for a potential PEEP change under conditions where the FiO2 suddenly changes, or seems to suddenly change, for reasons that might not reflect the correct patient conditions under which to allow change of the PEEP setting, such as, for example, as may result from erroneous or inaccurate measurement of SpO2 or erroneous determination of current FiO2. However, condition (2) may include a smaller time period requirement in order to allow for a potential PEEP change to provide an enhanced or prioritized ability to rapidly respond to patient desaturation, which can indicate a critical and urgent emergency warranting urgent increased patient support, which may include a PEEP increase, for example.

In some embodiments, PEEP change eligibility rules may include that, for a PEEP increase, the PEEP has not changed for at least a first period of time, and for a PEEP decrease, that the PEEP has not changed for at least a second period of time. The first and second periods of time may be different. As such, in some embodiments, particular time thresholds are set, which thresholds must be met in order to increase or decrease PEEP, even if a PEEP increase or decrease is otherwise indicated. Furthermore, the thresholds may differ based on whether the indicated PEEP change is an increase or a decrease. As such, in some embodiments, PEEP selection rules, or aspects thereof, may be referenced as part of or related to use of PEEP change eligibility rules.

Additionally, in some embodiments, PEEP change eligibility conditions may include that one or more measures of the patient's hemodynamic status, as may, for example, be determined at least in part based on a blood pressure measurement, is of at least a predetermined level, which blood pressure measurement may, in various embodiments, be obtained by the ventilator or another system or device in communication with the ventilator, such as a separate critical care monitor, defibrillator, or other device, for example. In some embodiments, this condition may ensure that the patient's hemodynamic status is sufficient to warrant a change in PEEP.

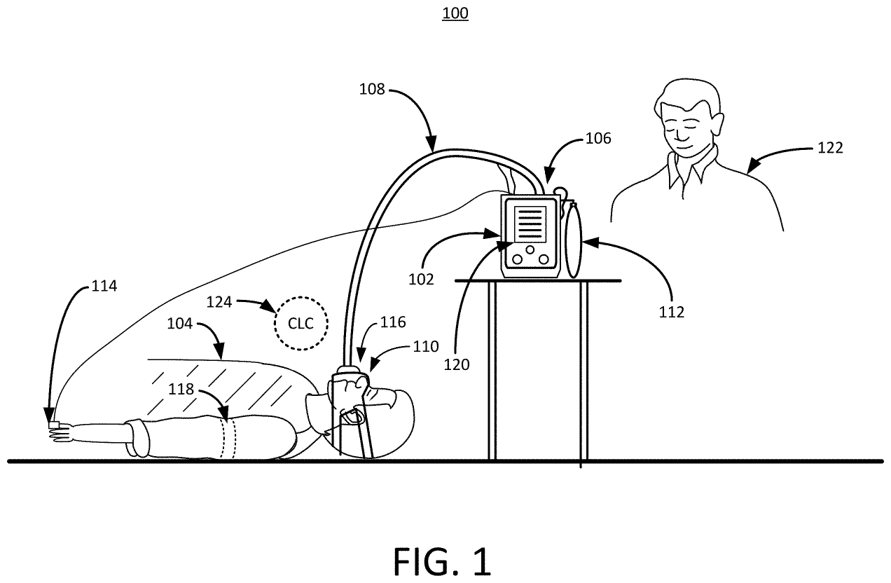

FIG. 1 illustrates an example system 100 incorporating closed loop control, as conceptually represented by broken circle 124, in mechanical ventilation of a patient 104, in accordance with embodiments of the present disclosure. While a portable (e.g., may be carried or practically carried or taken by a care provider to a patient at an out-of-hospital or pre-hospital location) ventilator 102 is shown, embodiments are contemplated in which a non-portable ventilator is provided or utilized. When operating, the portable ventilator 102 may provide breathing gas to a patient 104 via a gas delivery apparatus 106, including a patient circuit 108 that includes a facemask 110, though, in some embodiments, ventilation may be provided via intubation rather than via a facemask. A user 122 is also shown. The portable ventilator 102 may be coupled with an oxygen source, such as an oxygen tank 112, and may also be coupled or integrated with other devices. The portable ventilator 102 may also include various sensing, measuring, computerized, electrical, mechanical, coupling and output components. As depicted, the portable ventilator 102 includes an oximetry sensor 114, such as a pulse oximeter or other sensor for providing a direct or indirect measurement, estimation or indication of oxygen saturation (SpO2) or other blood oxygen content or concentration related parameter, a capnographic sensor 116 or capnograph, and a blood pressure sensor/monitor 118, and may also include one or more flow sensors such as pneumotachometers, or pressure sensors, among other things.

The portable ventilator 102 includes a display and user interface 120 that may provide data relating to various patient physiological, respiratory and ventilation related parameters, and may include other output or presentation components, such as a speaker. In some embodiments, the display and user interface 120, or other output devices, may provide a display that is integrated to include data relating to operation of other coupled devices that may also be in use with the patient, such as, for example, a defibrillator. The display and user interface 120 may also allow user interaction, including to obtain or display data, change settings, accept suggested or recommended settings changes, or view or respond to alarms or alerts, among other things.

The portable ventilator 102 is capable of providing closed loop control 124 of one or more ventilation or patient related parameters, as described with regard to various embodiments herein, such as may include use of a controller that may execute one or more algorithms in implementing methods described herein. The location or setting depicted in FIG. 1 may include, among other things, a pre-hospital or non-hospital location or setting, which could include an emergency vehicle setting or other venue or location. The portable ventilator 102 may, for example, be stored at or near the location or setting, or may be carried or taken to the location or setting to provide emergency care to the patient 104.

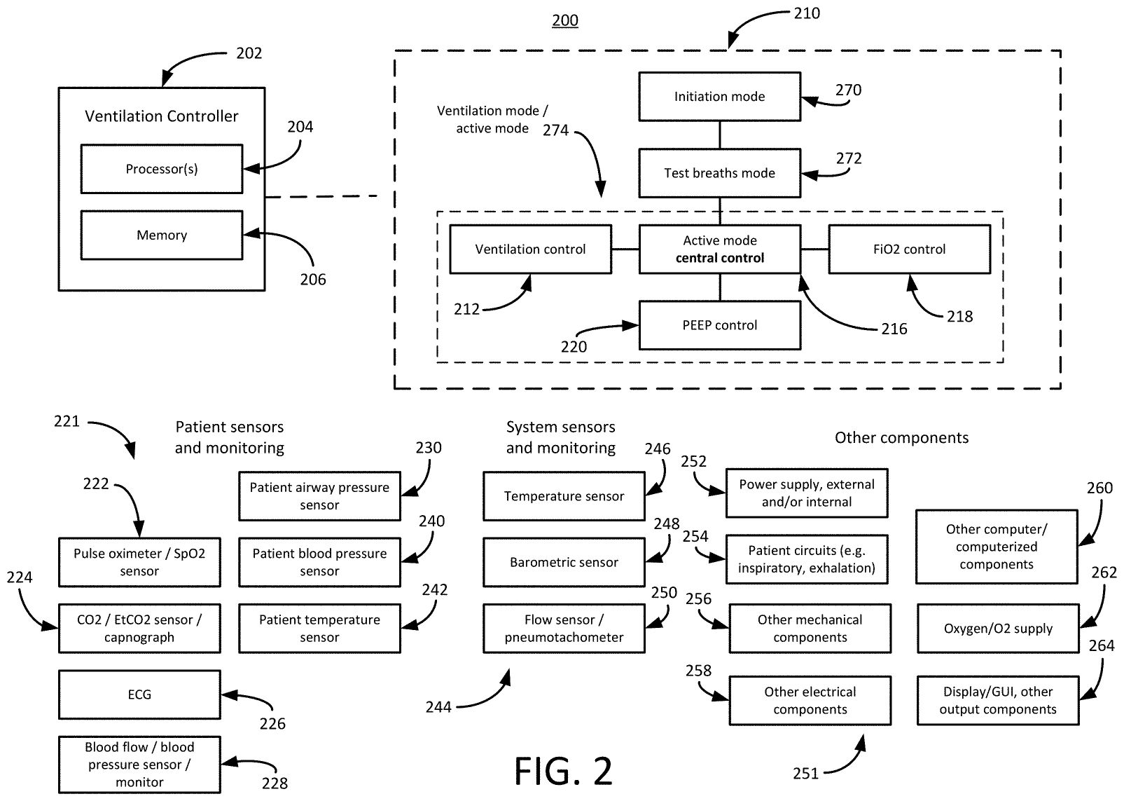

FIG. 2 illustrates an example portable ventilation system or ventilator 200, incorporating closed loop control, in accordance with embodiments of the present disclosure.

The ventilation system or ventilator 200 may include various components, such as patient sensors and monitoring components 221, system sensors and monitoring components 244, and other components 251. The patient sensors and monitoring components 221 may include an oximetry sensor 222, such as a pulse oximeter or other sensor for providing a direct or indirect measurement, estimation or indication of oxygen saturation (SpO2) or other blood oxygen content or concentration related parameter, a CO2/EtCO2 sensor/capnograph 224, an ECG component 226 and a patient blood flow sensor 228.

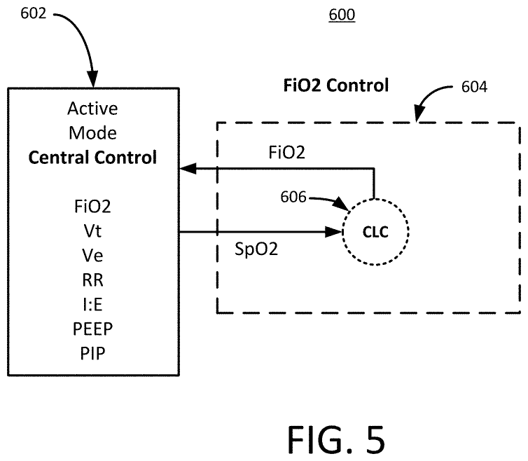

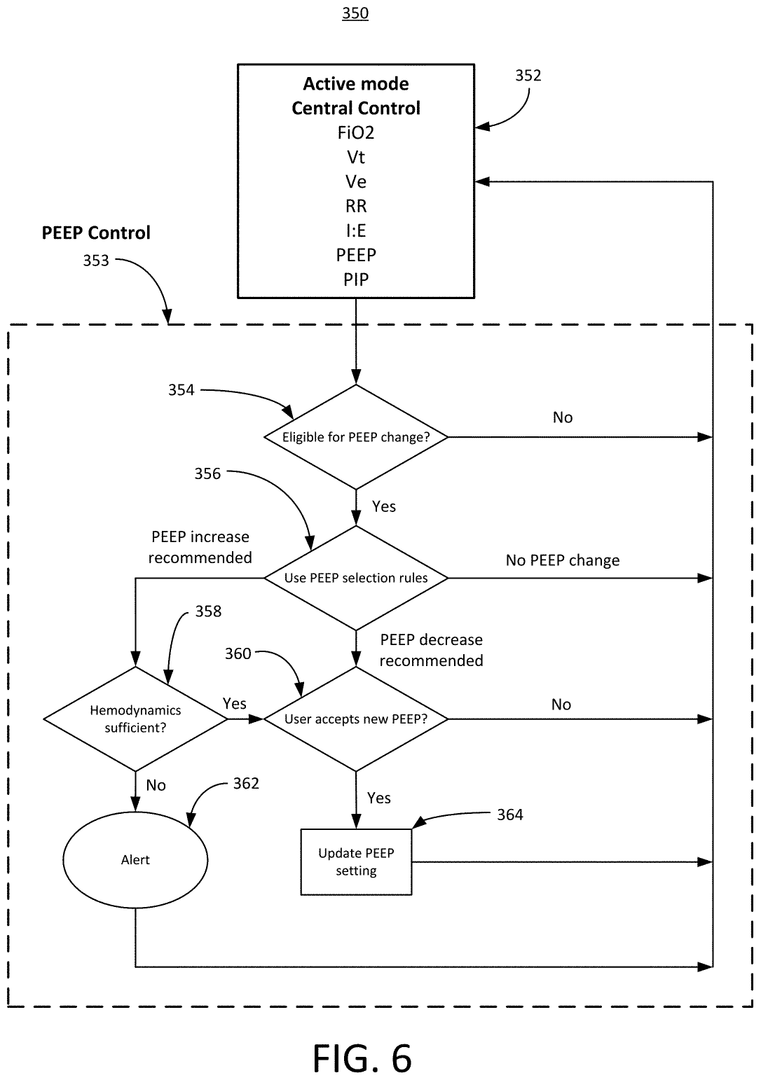

Various combinations of these components are used in performing closed loop control of ventilation parameters according to various embodiments, such as, for example, while ventilation is being provided to a patient via a gas delivery apparatus 106, and using a facemask 110, as depicted in FIG. 1, coupled with the patient, or intubation. For example, as described with reference to FIG. 5, measured patient SpO2 (such as may be measured, for example, using an oximetry sensor 114, as depicted in FIG. 1, such as a pulse oximeter coupled with the patient) is used in determining FiO2 updates. As described with reference to FIG. 4A, measured EtCO2 (such as may be measured using a capnographic sensor 116 or capnograph, as depicted in FIG. 1, coupled with the patient) is used in determining parameter updates, including Vt, Ve, RR and PIP, such as in connection with patient hypercapnia, hypocapnia and normocapnia conditions. As described with reference to FIG. 6-7, patient hemodynamics, such as measures of blood flow including systolic blood pressure, may be used in connection with PEEP change eligibility (such as may be measured, for example, using a blood pressure sensor or monitor 118, as depicted in FIG. 1, coupled with the patient).

The system sensors and monitoring components 244 may include a temperature sensor 246, a barometric sensor 248 and flow sensor(s) 250 such as those that utilize pneumotachometer(s), or other types of sensed parameters. Other components 251 include an external and/or internal power supply 252, patient circuits 254, including inspiratory and exhalation circuits, other mechanical components 256, other electrical components 258, other computer or computerized components, such as one or more central processing units, processors and memories 260, and on oxygen/O2 supply 262, and a display/GUI and potentially other output components 264.

Also included is ventilation control software 210 with closed loop control capability, which may be stored in whole or in part in the one or more memories 206 of the ventilation controller 202 and executed in whole or in part by the one or more processors 204 of the ventilation controller 202.

The ventilation controller 202 software includes components including an initiation mode engine 270, a test breaths mode engine 272 and a ventilation mode/active mode engine 274. Various other software may be included, including software that may be stored or executed in whole or in part outside of the portable ventilator or ventilation system 200.

The active mode engine 274 includes a ventilation control engine 212, an FiO2 control engine 218 and a PEEP control engine 220, where each or some of these engines 212, 218, 220 may be in coordination with a central control engine 216 or operate independently, and each may or may not also be in coordination with each other or some of the other engines. Different aspects of the ventilation closed loop control may be activated, for example, ventilation control 212, FiO2 control 218, and/or PEEP control 220 may be activated alone or in combination. In some embodiments, FiO2 closed loop control is activated without PEEP closed loop control, such that PEEP is manually adjusted by the user; or PEEP closed loop control is activated without FiO2 closed loop control, such that FiO2 is manually adjusted by the user; or ventilation closed loop control may be activated where FiO2 and PEEP are based manually adjusted by the user.

In some embodiments, FiO2 closed loop control can provide advantages including minimizing the time that a patient's SpO2 is below or substantially below a target value, providing rapid oxygenation response to patient SpO2 desaturation events and helping prevent patient hypoxemia and hyperoxymia.

It is noted that, while the modes and engines are depicted separately for conceptual purposes, they may be implemented in a combined, integrated or different manner, such as embodiments in which aspects of the roles of each of the modes or engines are distributed differently or combined in various ways. Moreover, other conceptual frameworks may also be utilized in various embodiments.

In some embodiments, the ventilator system or ventilator 200 may be operated in one of several overall system modes of operation. One (or several) such system modes may include closed loop control of FiO2, PEEP, and ventilation, which, for convenience and without limitation, may be referred to herein as ventilation/PEEP/FiO2 controller (VPFC) system mode. In some embodiments, operation in VPFC system mode includes initiation mode 270, test breaths mode 272 and active mode (or ventilation mode) 274, which modes may, in some embodiments, occur in order and represent phases of operation in VPFC system mode. In some embodiments, after turning on the ventilator 200, the user may select VPFC system mode. However, embodiments are contemplated in which VPFC system mode is entered into or begun without user selection or input.

In various embodiments, various types of closed loop control, such as VPFC mode, can be started or engaged, as well as stopped or disengaged, in different ways. In some embodiments, VPFC or other aspects of closed loop control may start automatically, such as at the onset of active mode ventilation. In some embodiments, the ventilator 200 may include a control component, such as a physical button, control knob or GUI component, which the user may press, engage or actuate in order to start, select or turn on VPFC or a particular aspect of closed loop control. For example, in some embodiments, the user may be provided with options to select and initiate, for example, VPFC, closed loop control of FiO2 but manual PEEP control or adjustment, or closed loop control of PEEP but manual FiO2 control or adjustment. Furthermore, in some embodiments, the user may be provided with options to start, stop or re-start various types of closed loop control at different times, so that VPFC or other aspects of closed loop control are available, but also subject to disengagement, and so are made to be essentially on-demand. Also, as mentioned previously, in some embodiments, during operation of aspects of closed loop control, the user may be able to change a particular setting or settings, such as FiO2 or PEEP, and then closed loop control may resume from that point and initially with that setting or those settings. Furthermore, in some embodiments, a user may be provided with an option to stop or disengage VPFC or some other aspect of closed loop control, and later the user may restart it. Still further, in some embodiments, multiple or different users may oversee or operate the ventilator, such as at different times, and each user may take different actions.

In some embodiments, for example, a user, such as a minimally trained user, may initiate active mode ventilation in VPFC mode (or another aspect of closed loop control). At some point during active mode ventilation, that user or another user, such a more trained user, may change from VPFC (or another aspect of closed loop control) to manual, or more manual, operation. Alternatively, active mode ventilation may be initiated without VPFC or some other aspect of closed loop control, and VPFC or some other aspect of closed loop control may be started or engaged later, such as if the user becomes distracted (or during the period of user distraction), or by a less trained user who may later arrive at the scene, for example.

The following provides an exemplary overview of operation in VPFC system mode, according to some embodiments. Further details, regarding various aspects and in various embodiments, are provided with reference to later figures.

In some embodiments, at the start of initiation mode 270, or after the user selects VPFC system mode, the user may be prompted (via a display of the ventilator 200) to enter the patient's gender and height. From this, the controller 202 may determine an associated bodyweight, which may be called the patient's “predicted” bodyweight (PBW). However, the determined bodyweight may not be the patient's actual bodyweight, and may represent an ideal bodyweight, approximated, typical or other associated bodyweight to be used, for example, in determination or optimization of certain ventilation settings. Predicted bodyweight may be used, as opposed to actual bodyweight, for example, since actual bodyweight may fluctuate substantially from person to person, and even between individuals of similar characteristics such as height and gender, due to different amounts of muscle and fat from person to person, while lung volume, capacity and function remain relatively unaffected by such factors. As such, in some embodiments, PBW may be used at least in part as an indicator associated with an individual's respiratory capacity, and may generally be a better indicator than actual patient bodyweight.

During initiation mode 270, the controller 202 may utilize the PBW in determining one or more ventilation parameter settings to be used for one or several operational breaths to be delivered to the patient during initiation mode, which may be utilized, for example, in confirming correct operation of the ventilator 200, which may include confirming that there are no leaks, or significant leaks, in the circuit. For example, PBW may be used in setting initial Vt, such as may be set as Vt (in ml)=A*PBW (in kg), where A may be, e.g., 5, 6, 7, 8 or 9 (in ml/kg). Furthermore, for example, initial Ve (in ml/min) may be set as B*PBW, where B (in ml/(min*kg) may be, e.g., 80, 90, 100, 110, 120 or 150.

Once initiation mode 270 is successfully completed, the ventilator 200 may proceed to test breaths mode 272. During test breaths mode 272, one or more test breaths are delivered to the patient and respiratory data is obtained. The respiratory data is used in determining one or more patient respiratory parameters, such as estimated patient respiratory system compliance (Crs) and, in some embodiments, one or more additional parameters, such as estimated patient respiratory system resistance (Rrs). In some embodiments, if Crs cannot be estimated or sufficiently estimated then a default value, such as 100 ml/cm H2O may be used. The determined patient Crs is then used in determining an initial peak inspiratory pressure (PIP(0)) setting to be used at the start of active mode 274.

In some embodiments, active mode 274 may include closed loop control of FiO2 and PEEP, and possibly one or more other parameters, as described in further detail with reference to later figures herein. During active mode 274, central control 216, in coordination with ventilation control 212, FiO2 control 218 and PEEP control 220, sets or adjusts ventilation settings including FiO2, PEEP, PIP, target tidal volume (Vt), target minute volume (Ve), respiratory rate (RR), inspiratory:expiratory ratio (I:E). However, in some embodiments, one or more of ventilation control 212, FiO2 control 218, PEEP control 220, or other components, may set or adjust, or participate in the setting or adjusting of, certain of those or other ventilation settings.

In some embodiments, the controller 202 continuously monitors for autoPEEP (PEEPi), which may result from incomplete emptying of the lungs when an expiratory phase is too short, and may create risk of dynamic hyperinflation of the lungs. In some embodiments, PEEPi is detected and measured based on detection and measurement of a gas flow existing at the end of an exhalation period. When PEEPi is detected, the controller 202 may, in some embodiments, continuously adjust I:E to increase expiratory, or may decrease Vt/kg, which can reduce or eliminate PEEPi.

As described in detail herein, the controller 202 may utilize a target SpO2, such as 94% (however, in various embodiments, the target SpO2 may be, e.g., between 93%-98%, between 93%-96% or between 96%-98%). On a continuous basis, actual measured patient SpO2 may be used as input in adjusting the FiO2 setting, which changed FiO2 setting or adjustment may further be used in adjusting the PEEP setting.

In some embodiments, determined parameters including patient end-tidal carbon dioxide (EtCO2), a determined patient airway pressure waveform (P), patient airway flow waveform (Vdot), and volume waveform (V) may be used in determining active mode initial settings for Vt, Ve, PIP, RR and I:E, and/or, in some embodiments, parameters can be used that are derived at least in part from the Vdot and V waveforms, which can include plateau pressure, driving pressure, Crs and Rrs. Furthermore, the controller 202 may continuously use determined patient Crs and Rrs in determining adjustments to Vt, such as may maintain Vt at a safe level. The controller 202 may also continuously adjust RR, such as to maintain Ve at a constant level when Vt is adjusted, where Ve=Vt*RR.

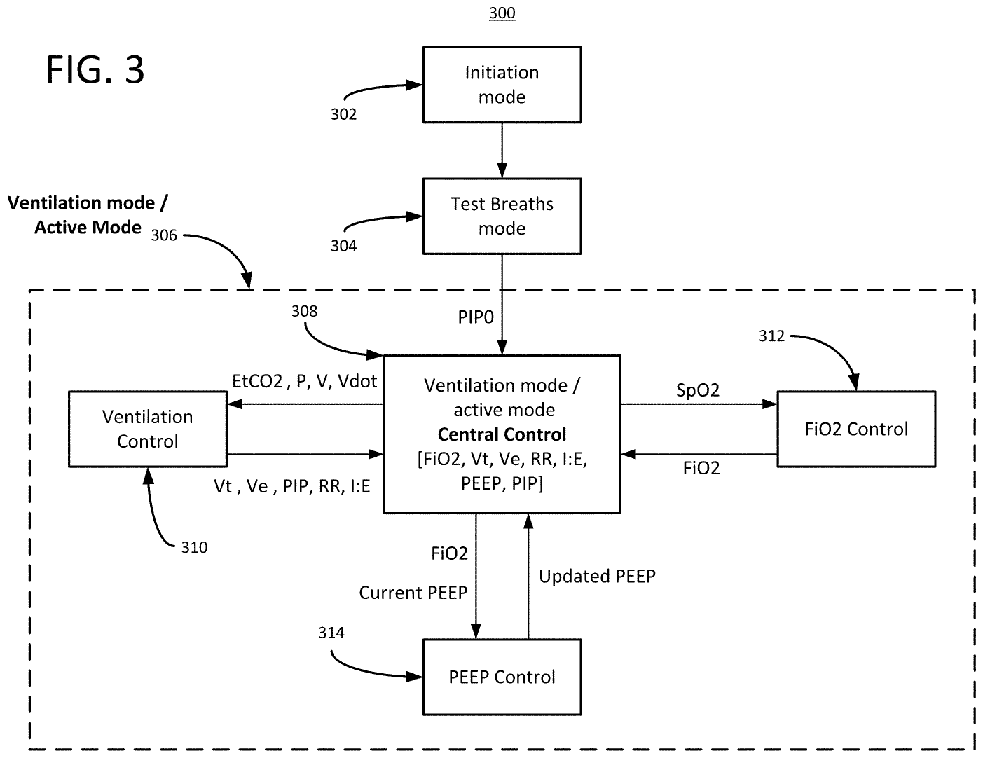

FIG. 3 is a block diagram illustrating example conceptual or software architecture-related aspects 300 of a ventilation system incorporating closed loop control, in accordance with embodiments of the present disclosure, which may be implemented by a controller, such as controller 202 as depicted in FIG. 2. An initiation mode 302, a test breaths mode 304 and an active mode 306 are shown. During active mode 308, the controller 202 may cause delivery of ventilation to the patient, such as, for example, via a gas delivery apparatus 106 and using a facemask 110, as depicted in FIG. 1, coupled with the patient, or intubation, using ventilation parameters that are continuously updated, which may include FiO2 and PEEP. It is to be understood that other embodiments are contemplated that do not use the particular exemplary modes described herein.

In initiation mode 312, the user may be prompted to enter the patient's gender and height, which may be used in determining the patient's PBW. Based on the determined patient's PBW, several ventilation parameter settings may be determined for use in test breaths mode 304.

In various embodiments, in initiation mode, various calculations may be utilized to determine test breaths parameter settings. In one example, in porcine use and studies (examples of which are provided herein with reference to later figures), Vt (in ml) may be calculated as 10*actual weight in kg, Ve (in ml/min) as actual weight in kg*140, and RR as Ve/Vt, where RR may be rounded to the next higher integer value. However, various other calculations and values may be used in various embodiments.

In another example, in human use, the following calculations may be utilized. The PBW (in kg) may be calculated as C+D*(height in cm-E), where C may be, e.g., between 35-60, D may be, e.g., between 0.80 and 1.00, and E may be, e.g., between 140-160, and where, in some embodiments, at least C may be slightly lower for a female than for a male. For Vt greater than or equal to, e.g., a value between 150-250 ml, rounding may be performed to, e.g. the nearest 1-10 ml, 3-7 ml, and for Vt less than or equal to, e.g., a value between 15-200 ml, rounding is performed to, e.g., the nearest 0.5-5 ml, 0.5-3 ml. However, various other calculations and values may be used in various embodiments, such as various calculations that are based on, or functions of, height, gender and/or one or more other patient physical or health-related characteristics.

In some embodiments, some or all of the results of the calculations made in initiation mode 302 are displayed to the user. The user may, for example, be prompted to accept the determined parameters and then couple the patient to the ventilator.

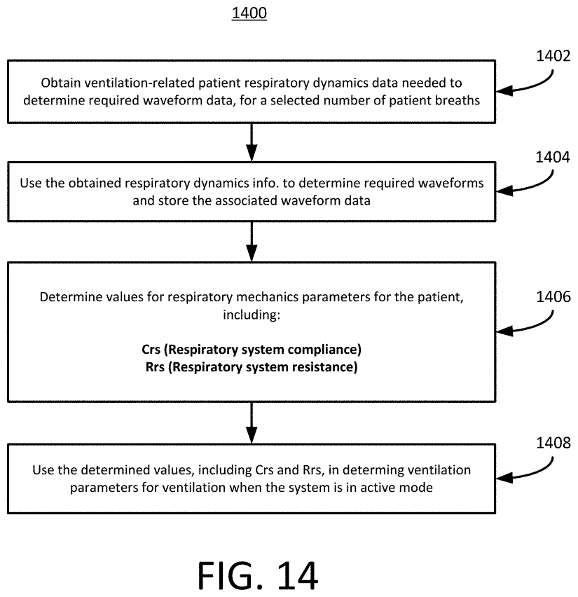

In some embodiments, following completion of initiation mode 302, the system 300 proceeds to test breaths mode 304. In test breaths mode 304, the system 300 may deliver one or several ventilation test breaths, such as, for example, via a gas delivery apparatus 106 and using a facemask 110, as depicted in FIG. 1, coupled with the patient, or intubation, based on which the system 300 may determine one or more patient parameters and/or particular ventilator settings to utilize at the start of active mode 306. For example, in some embodiments, in the test breaths mode 304, a patient Crs, such as an estimated patient Crs, is determined and used in determining a PIP(0) ventilator setting. As described further with reference to FIG. 14, in some embodiments, Crs may be calculated or estimated using patient respiratory dynamics data and the equation of motion for the respiratory system. Furthermore, in some embodiments, in test breaths mode 304, the following particular parameter settings may be utilized: I:E=1:3, PEEP=5 cm H2O, FiO2=0.5 (or 50%). However, in various embodiments, other initial settings may be used.

Following successful completion of the test breaths mode 304 and determination of the patient Crs, the system 300 may proceed to active mode 306. In some embodiments, the system may proceed to active mode 306 even if patient Crs cannot be determined. For example, in some embodiments, if Crs cannot be determined, a default Crs value may be utilized, such as a Crs value of 50-150 ml/cm H2O, 70-130 ml/cm H2O, 80-120 ml/cm H2O, 90-110 ml/cm H2O, for example. In some embodiments, the default value may be determined to be relatively high, since, in some embodiments, that will result in relatively small changes in a PIP correction value (Pcorr) (as described with reference to FIG. 4A), thus providing a relatively conservative approach.

In some embodiments, during active mode 306, the system 300 delivers continuously adjusted ventilation to the patient, which may include closed loop control of one or more patient and/or ventilation related parameters.

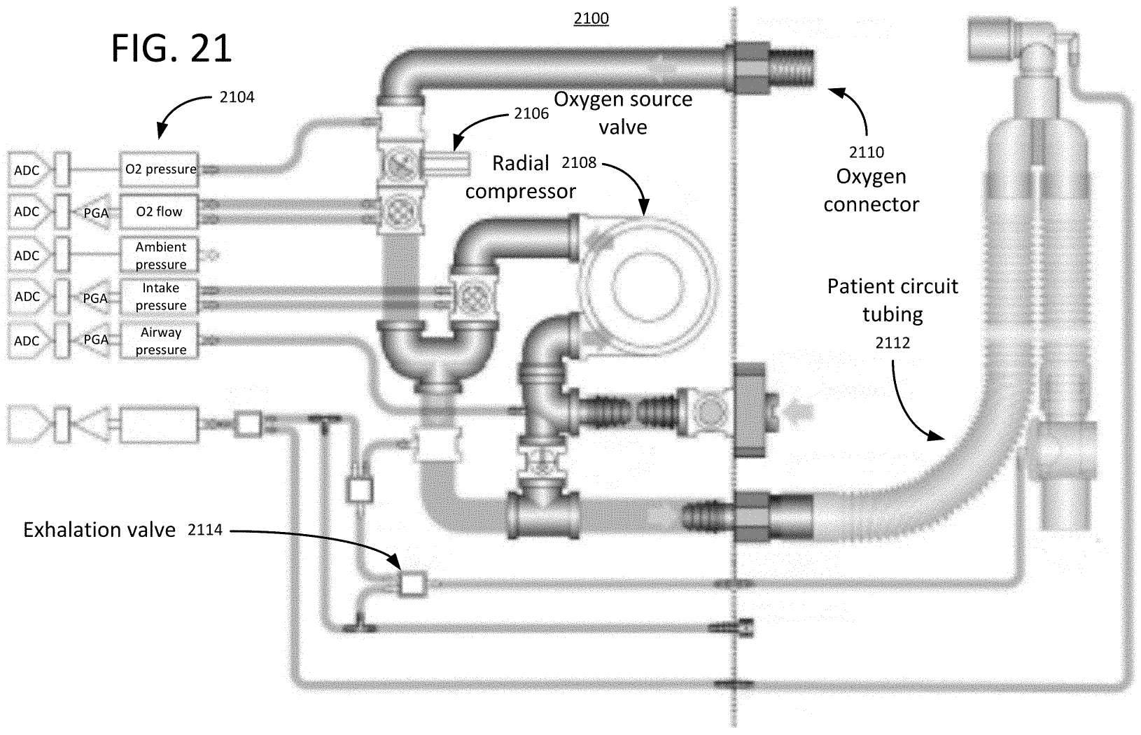

In some embodiments, during active mode 306, a number of ventilation parameters are continuously adjusted, including FiO2 (such as, for example, via actuation of an oxygen source valve 2106, as depicted in FIG. 21) and PEEP (such as, for example, via actuation of an exhalation valve 2114, as depicted in FIG. 21). Other parameters that are adjusted during ventilation mode 308 in a continuous manner may include PIP, Vt, Ve, RR and I:E. In some embodiments, FiO2 is continuously adjusted based on a target patient oxygenation level, such as an SpO2 of 94%. In some embodiments, FiO2 starts at 21% (or, e.g., 21-25%). However, some embodiments, a user may select an initial FiO2 setting, such as between 21%-100%, for example.

On a continuous basis, central control 308 may provide data to ventilation control 310, including current EtCO2 as well as pressure (P) and volume (V) waveforms, and/or parameters derived at least in part from the P and V waveforms. Ventilation control 310 may use that data, potentially in addition to other data, in determining values for Vt, Ve, PIP, RR and I:E, which it then sends to central control 308. Central control 308 may then implement any appropriate adjustments to Vt, Ve, PIP, RR and I:E settings based at least in part on the sent values, as further described with reference to FIG. 4A.

Furthermore, on a continuous basis, central control 308 may provide data to FiO2 control 312, including current patient SpO2. FiO2 control 310 may use that data, potentially in addition to other data, in determining a value for FiO2, which it then sends to central control 308, as further described with reference to FIGS. 5 and 12A. Central control 308 may then implement any appropriate adjustment to the FiO2 setting based at least in part on the sent values. Adjustment of the FiO2 setting may be accomplished via appropriate actuation of an oxygen source valve, or by adjusting an oxygen concentration from an oxygen supply, or in other ways.

Still further, on a continuous basis, central control 308 may provide data to PEEP control 314, including the current FIO2 and current PEEP. PEEP control 314 may then use that data, potentially in addition to other data, in determining an updated value for PEEP, which it then sends to central control 308, as described further with reference to FIGS. 6-8. Central control 308 may then implement any appropriate adjustment to the PEEP setting based at least in part on the sent value. Adjustment of the PEEP setting may be accomplished via appropriate actuation of an exhalation valve, for example.

It is to be understood that, while central control 308, ventilation control 310, FiO2 control 312 and PEEP control are described separately and communication with each other, in some embodiments, some or all of these may be combined or integrated, or may function independently. In such embodiments, communication between combined or integrated components may be less or may be unnecessary.

FIG. 4A is a flow diagram illustrating an example method 400 of operation of a ventilation control engine 401 of a ventilation system incorporating closed loop control, in accordance with embodiments of the present disclosure. The method 400 is conceptually depicted as being implemented by ventilation control 401, in coordination with central control 402.

In some embodiments, particular ventilation parameters may be adjusted, such as, for example, via ventilation delivered via the gas delivery apparatus 106 and using a facemask 110, as depicted in FIG. 1, coupled with the patient, or intubation, within particular ranges, based at least in part on continuously monitored EtCO2 (such as may be monitored, for example, using a capnographic sensor or capnograph 116, as depicted in FIG. 1), which may be indicative of patient hypercapnia, hypocapnia or normocapnia.

CO2 arises in expelled gas from a patient as a byproduct of cellular metabolism and aerobic respiration. CO2 is carried by the blood to, and then expelled by, the lungs. Hypercapnia, as indicated by high EtCO2, can indicate that the lungs are not able to efficiently eliminate or remove CO2 from the body. This can lead to an accumulation or increased concentration of CO2 at the end of each breath, causing an elevated EtCO2 and a hypercapnia condition.

When hypercapnia is detected, a goal of ventilation may be to assist the patient's respiration as needed to allow a sufficient rate of removal of CO2 from the lungs, so as to reduce EtCO2 and return to a normocapnia condition. This may be done by increasing Ve, within a range, where the resulting increase in the volume of gas moved per minute increases the rate of elimination of CO2 and thus reduces EtCO2. Step 420 of FIG. 4A, as described in more detail below, reflects this.

When hypocapnia is detected, this can be indication that more assistance or support is being provided to the patient than is needed in removing CO2, and may indicate that the patient is more efficiently breathing and eliminating CO2 on his or her own, or could indicate patient hyperventilation. Ve may be appropriately decreased (such as, for example, via appropriate control of a gas delivery apparatus 106, as depicted in FIG. 1), providing less assistance to the patient and leading to an increase of EtCO2 and return to a normocapnia condition. Step 415 of FIG. 4A, as described in more detail below, reflects this.

Since Ve=Vt*RR, Ve may be increased (such as, for example, via appropriate control of a gas delivery apparatus 106, as depicted in FIG. 1) by increasing Vt, RR or both, and, conversely, Ve may be decreased by decreasing Vt, RR or both. In some embodiments, whether one or both of Vt or RR is changed, and by how much, may depend on the current Vt. For example, in some embodiments, Vt may be restricted to particular range, such as 100%-150% of a predicted Ve (as shown in steps 420 and 415, described further below). In various embodiments, either or both of Vt and RR may be restricted to particular ranges or thresholds, which may be used in determining whether to adjust either or both, and by how much. As one of many possible examples, in some embodiments, assuming that Ve is to be increased, if Vt is less than a particular threshold value, such as 8 ml/kg (or, e.g., 7 or 9 ml/kg), then Vt may be increased, such as by 1 ml/kg (or, e.g., a fraction of 1 ml/kg or between 1-2 ml/kg), and RR may also be slightly adjusted so as to result in a desired increase in Ve, which may be, e.g., a percentage increase in Ve, such as 10% (or, e.g., between 5-10% or between 10-15%). As another example, in some embodiments, assuming that Ve is to be decreased, to achieve a desired decrease in Ve, if RR is above a particular threshold value, such as 14 BPM (or, e.g., 12-14 BPM or 14-16 BPM), then RR may be decreased, otherwise Vt may be decreased (or both).

However, when ventilation parameter adjustments are made to lead to return to, or maintain, normocapnia, these changes must not cause any particular ventilation parameter to move outside of a safe or permitted range—in such cases, an alert may be provided to the user. For example, as reflected in steps 421 and 412 of FIG. 4A, as described further below, if Ve moves outside of a particular range, a low or high Ve alert may be provided or displayed to a user or care provider, as appropriate.

Furthermore, a plateau pressure or driving pressure that is too high can cause barotrauma that can injure a patient's lungs (where driving pressure is equal to plateau pressure minus PEEP, and can be estimated as Vt/Crs). As such, if the plateau pressure or driving pressure is above a particular threshold, it may be decreased by decreasing Vt/kg accordingly. This is reflected in step 410 of FIG. 4A, as described below.

In FIG. 4, at step 404, it is determined whether a plateau pressure (or, in other embodiments, a driving pressure) is below, or equal to or below, a particular high plateau pressure threshold, such as, for example, 30 cm H2O (however, in various embodiments, the threshold may be, e.g., between 20-50 cm H2O). The purpose of this check is to ensure that the pressure applied to the patient is not too high. If the plateau pressure or driving pressure is sufficiently below the predetermined threshold, then the algorithm may proceed to the next step in assessing whether the patient is hypocapnic, normocapnic, or hypercapnic.

As depicted, at step 404, if the plateau pressure is not equal to or below the high plateau pressure threshold, then, it may be desirable to lower to tidal volume Vt so as to lower the pressure applied to the patient. However, before lowering the tidal volume Vt, then a further check performed at step 406, where the method 400 determines whether Vt/kg is above a particular low Vt/kg threshold, such as, for example, 4 ml/kg (however, in various embodiments, the low Vt/kg threshold may be, e.g., between 2-6 ml/kg).

At step 406, if Vt/kg is not above the low Vt/kg threshold, then, at step 408, a low Vt/kg alarm is triggered. If Vt/kg is above the low Vt/Kg threshold, then, at step 410, Vt is decreased, such as by 1 ml/kg (or, e.g., a fraction of 1 ml/kg, between 1-2 ml/kg, or more than 2 ml/kg). Additionally, in the embodiment depicted, at step 410, since Vt/kg is decreased, in order to maintain Ve without change, RR is increased as appropriate, given that Ve=Vt*RR.

At step 404, if it is determined that the plateau pressure is equal to or below the high plateau pressure threshold, then, at step 422, an EtCO2 parameter, or other carbon dioxide related parameter, is assessed. For example, the EtCO2 parameter may be an EtCO2 average over a time period including the current time, such as a one minute, partial minute or multiple minute EtCO2 average, which may be calculated as an average of multiple time-spaced individual measured EtCO2 values, for example.

At step 422, if the EtCO2 parameter is below a lower threshold (“hypocapnia”), such as 25 mm Hg (or, e.g., 20-50 mm Hg), then, at step 414, Ve is assessed. At step 414, if Ve is not greater than a predicted Ve, then, at step 421, a low Ve alert is triggered, since this may indicate that insufficient gas is being supplied to the patient. At step 414, if Ve is greater than the predicted Ve, then, at step 415, Ve is decreased, for example, such that the new Ve is Ve/F, where F is, e.g., between 1.05-1.15), so that a more appropriate amount of oxygenation support is provided. In some embodiments, Ve may not be decreased again for a predetermined interval at least, e.g., in minutes, 10-20, 20-30, 30-45 or 45-60. In some embodiments, reasons for the predetermined interval may include ensuring enough time for the current Ve to have sufficient or full physiological effect, which can prevent potentially over-decreasing Ve.

At step 422, if the EtCO2 parameter is above an upper threshold (“hypercapnia”), such as 40 mm Hg (or, e.g., 30-60 mm Hg), then, at step 416, Ve is assessed. If Ve is not less than, e.g., 1.4-1.6 predicted Ve, then, at step 412, a high Ve alert is triggered based on a concern that too much gas is being delivered to the patient. If Ve is less than, e.g., 1.4-1.6*the predicted Ve, then, at step 420, Ve is increased, for example, such that the new Ve will be the current Ve*G, where G is, e.g., 1.05-1.15, to provide additional support to the patient in clearing CO2 from the body. In some embodiments, Ve may not be increased again for a predetermined interval at least, e.g., in minutes, 10-20, 20-30, 30-45 or 45-60. In some embodiments, reasons for the predetermined interval may include ensuring enough time for the current Ve to have sufficient or full physiological effect, which can prevent potentially over-increasing Ve.

In some embodiments, an assessment, such as an algorithmic assessment of one or more aspects of an CO2 waveform (e.g., resulting from measured EtCO2 values over a recent period of time) may be used as a factor in determining whether to allow an increase or decrease in Ve. For example, in some embodiments, one or more aspects of the CO2 waveform (e.g., EtCO2 value) must meet one or more threshold conditions in order for Ve to be changed, even if an increase in Ve is otherwise indicated. For example, in some embodiments, a change to Ve may be permitted if the assessed quality of the CO2 waveform (e.g., EtCO2 value) reaches a certain threshold. For example, one or more algorithms, which may include one or more data fitting models, may be used to determine how close the measured CO2 waveform or EtCO2 value is to an associated predicted CO2 waveform or EtCO2 value. A quantitative measure of this closeness, as algorithmically determined, may be taken as an indication of the quality of the measured CO2 waveform. A change to Ve may only be permitted if the quantitative measure meets or exceeds a certain threshold value, for example.

Furthermore, in some embodiments, an increase or decrease in Ve may be proportional to the magnitude of the difference between the current measured EtCO2 and a high or low normocapnia threshold, whichever threshold is closer to the current measured EtCO2. This may be accomplished, for example, by including a proportionality term in the calculation of the new, adjusted Ve. For example, suppose that the normocapnia range is defined as 35-45 mm Hg. A proportionality term could be included such that a hypocapnic EtCO2 of 20 mm Hg will result in a greater decrease in Ve than a hypocapnic EtCO2 of 25, since the magnitude of the difference between 35 (the lower limit of the normocapnia range) and 20 is greater than the magnitude of the difference between 35 and 25. Similarly, a hypercapnic EtCO2 of 55 mm Hg will result in a greater increase in Ve than a hypercapnic EtCO2 of 50, since the magnitude of the difference between 45 (the upper limit of the normocapnia range) and 55 is greater than the magnitude of the difference between 45 and 50. For example, for a hypocapnic EtCO2, a decrease in Ve could be calculated as Ve/(F*F1), where F1 is a term that is proportional to the magnitude of the difference between the current measured EtCO2 and the upper limit of the normocapnia threshold. Similarly, for a hypercapnic EtCO2, an increase in Ve could be calculated as Ve*G*G2, where G2 is a term that is proportional to the magnitude of the difference between the current measured EtCO2 and the upper limit of the normocapnia threshold. Similarly, in various embodiments, other more complex proportionality terms may be used, as well as terms that differ depending on whether an increase in Ve or a decrease in Ve is being calculated. Furthermore, in some embodiments, characteristics of the EtCO2 waveform may also be calculated and factored into the new, adjusted Ve calculations, in instances in which Ve adjustment is indicated and permitted.

At step 422, if the EtCO2 parameter is determined to be between the lower threshold and upper thresholds (“normocapnia”), inclusive of the threshold values (or, in other embodiments, exclusive of one or more of the threshold values), such as 25-50 mm Hg (or, e.g., 20-60 mm Hg), then, at step 418, a PIP Correction Value (Peon) is determined, such as, for example, (target Vt−last Vt)/Crs (some of various possible examples of determination of Crs are described with reference to FIG. 14), but Pcorr may be limited to be within a particular range, such as, e.g., −4 to −2 cm H2O to 2 to 4 cm H2O. In some embodiments, this limitation may help avoid sudden changes in PIP that could create risk to the patient.

In some embodiments, if Ve was changed recently, the method 400 proceeds from step 422 to step 418 even if EtCO2 is outside of the normocapnia range, as well as proceeding to step 420 or 414, as appropriate, in order to update PIP as may be needed in view of the recently changed Ve.

Following step 418, at step 424, PIP is updated accordingly, such as, for example by calculating the new PIP as the current PIP+Pcorr. However, the new PIP may be limited to be within a particular range, such as, for example, between a minimum of PEEP+2 cm H2O and a maximum of a high alarm limit—5 cm H2O. In some embodiments, if the new PIP would fall outside of the range, then an alarm may be triggered.

In FIG. 4B, the method 440 is conceptually depicted as being implemented by ventilation control 462, in coordination with central control 442.

At step 444, it is determined whether the driving pressure (or, in some embodiments, plateau pressure) is below a threshold, such as, for example, 13 cm H2O (however, in various embodiments, the threshold may be, e.g., 11-15 cm H2O).

At step 444, if driving pressure is not below the threshold, then, at step 446, it is determined whether Vt/kg is above a threshold, such as 4 ml/kg. If not, then, at step 448, an alarm may be triggered, such as a low Vt/kg alarm. If so, then, at step 450, Vt/kg is decreased, such as by 1 ml/kg (however, in various embodiments, the decrease may be, e.g., a fraction of 1 ml/kg, or between 1-2 ml/kg).

At step 444, if driving pressure is below the threshold, then, at step 442, the EtCO2 parameter is assessed.

If, at step 442, the EtCO2 parameter is above the a threshold, such as, for example, 45 mm Hg-55 mm Hg, then, at step 454, Ve is increased, such that the new Ve will be the current Ve*1.1, for example (however, in various embodiments, the new Ve may be, e.g., Ve*1.05-1.15).

In some embodiments, at step 442, if the EtCO2 parameter is below a different, lower threshold, such as, for example, 30 mm Hg 4 ml/kg (however, in various embodiments, the lower threshold may be, e.g., between 25-35 ml/kg), then, at step 462, PIP is decreased by 1 cm H2O (however, in various embodiments, the decrease may be, e.g., a fraction of 1 cm, or between 1-2 cm). In some embodiments, if the current PIP=PEEP+2 (however, in various embodiments, the this may be, e.g., PEEP+one or more less than 2), then an alarm may be triggered. However, in some embodiments, however, step 462 is not included. In some such embodiments, if, at step 442, EtCO2 is below the threshold, then the method 440 proceeds to step 458 regardless of the value of EtCO2.

Steps 458 and 460 may be analogous to steps 418 and 420 of the method 400 of FIG. 4B.

FIG. 5 is flow diagram illustrating aspects of an example method 600 of operation of an FiO2 control engine 604 of a ventilation system incorporating closed loop control, in accordance with embodiments of the present disclosure. In some embodiments, FiO2 control 604 may be implemented by a controller using a Proportional-Derivative (PD) control loop with no Integral (I) term, updating at the cycle interval frequency. However, other embodiments may use a Proportional-Integral-Derivative (PID) control loop, or another calculation, algorithm or model entirely.

In some embodiments, there may be a minimum error (e.g., difference between current SpO2 and target SpO2, or average of such differences for multiple recent SpO2 values, as described with reference to FIG. 12), that may trigger an FiO2 change, such as, e.g., a fraction of 1%, 1%, 1-2%, 2-3%, 3-5%, 5-10%, 10-15% or 15-20%. Furthermore, in some embodiments, in the event that a fault condition is detected affecting FiO2 determination (e.g., no 02 source is detected, or no probe is detected coupled to the patient), an alarm may be triggered.

As depicted, Central Control 602 may provide data to FiO2 control 604, including current measured patient SpO2 (such as may be measured, for example, using an oximetry sensor 114, as depicted in FIG. 1, such as a pulse oximeter coupled with the patient). FiO2 control 604 may use that data, potentially in addition to other data, in determining a value for FiO2, which it then sends to central control 602. Central control 308 may then determine and implement any appropriate adjustment to the FiO2 setting based at least in part on the sent values (such as, for example, via appropriate actuation of an oxygen source valve 2106, as depicted in FIG. 21). As depicted, FiO2 control 604 may include closed loop control aspects, as conceptually depicted by feature 606, such as may be used, for example, in regulation of FiO2 based at least in part on a target patient SpO2, such as an SpO2 of 94%, for example. Further detail regarding some embodiments of operation of FiO2 control are provided with reference to FIGS. 12-13 herein.

FIG. 6 is a flow diagram illustrating aspects of a simplified example method 350 of operation of a PEEP control engine 353 of a ventilation control system incorporating closed loop control, in accordance with embodiments of the present disclosure. More detailed example methods are provided with reference to FIGS. 7A and 7B.

The method 350 of FIG. 6 is conceptually depicted as being implemented by ventilation control 353, in communication with central control 352.

In some embodiments, generally, for PEEP to be changed, the PEEP change eligibility rules must indicate that PEEP is eligible for change (whether increase or decrease) and the PEEP selection rules must indicate an increase or decrease in PEEP, potentially among other conditions. In some embodiments, other conditions may include, for an increase in PEEP, that the patient's hemodynamics are evidenced as sufficient. In some embodiments, other conditions may include, for example, that the user accepts a recommended change.

At step 354, the method 350 determines whether PEEP is eligible for change. If not, then PEEP is not changed. If PEEP is eligible for change, then the method 350 proceeds to step 356, at which the PEEP selection rules are used to determine whether a PEEP increase, PEEP decrease, or no PEEP change is indicated. Details regarding an embodiment of PEEP selection rules are provided herein with reference to FIG. 8. It is noted that, while PEEP eligibility determination is depicted as occurring entirely before PEEP selection rule usage, embodiments are contemplated in which that is not the case. For example, in some embodiments, aspects of PEEP eligibility determination may occur before and after PEEP selection rule usage.

At step 354, if it is determined that PEEP is eligible for change, then the method 350 proceeds to step 356. At step 356, it is determined whether, according to the PEEP selection rules, a PEEP increase, a PEEP decrease, or no change in PEEP is indicated. If no PEEP change is indicated, then PEEP is not changed.

If, at step 356, it is determined that, according to the PEEP selection rules, a PEEP increase is recommended, then, at step 358, it is determined whether one or more measures of the patient's hemodynamics is sufficient, such as may be measured, for example, using a blood pressure sensor or monitor 118, as depicted in FIG. 1, coupled with the patient. For example, in some embodiments, a parameter associated with the patient's blood pressure measure may be utilized. For example, systolic blood pressure (SBP) may be required to be at or above a particular threshold, such as 90 mm Hg (or, e.g., 85-95 mm Hg), or mean arterial pressure (MAP) may be utilized, and may be required to be above a particular threshold, such as 60 mm Hg (or, e.g., 50-70 mm Hg), or another parameter may be utilized.

At step 358, if the patient's hemodynamics are determined or evidenced not to be sufficient, then, at step 362, an alert (e.g., a low systolic blood pressure alert) is displayed and PEEP is not changed. At step 358, if the patient's hemodynamics are determined or evidenced to be sufficient, then, at step 360, the user is prompted to accept the recommended new, increased PEEP. If the user accepts, then, at step 364, PEEP is increased accordingly (such as, for example, via appropriate actuation of an exhalation valve, as depicted in FIG. 21). If the user does not accept, then PEEP is not changed. It is noted that, in other embodiments, user acceptance of a PEEP change is not required and the check can be performed automatically without requiring user intervention or confirmation.

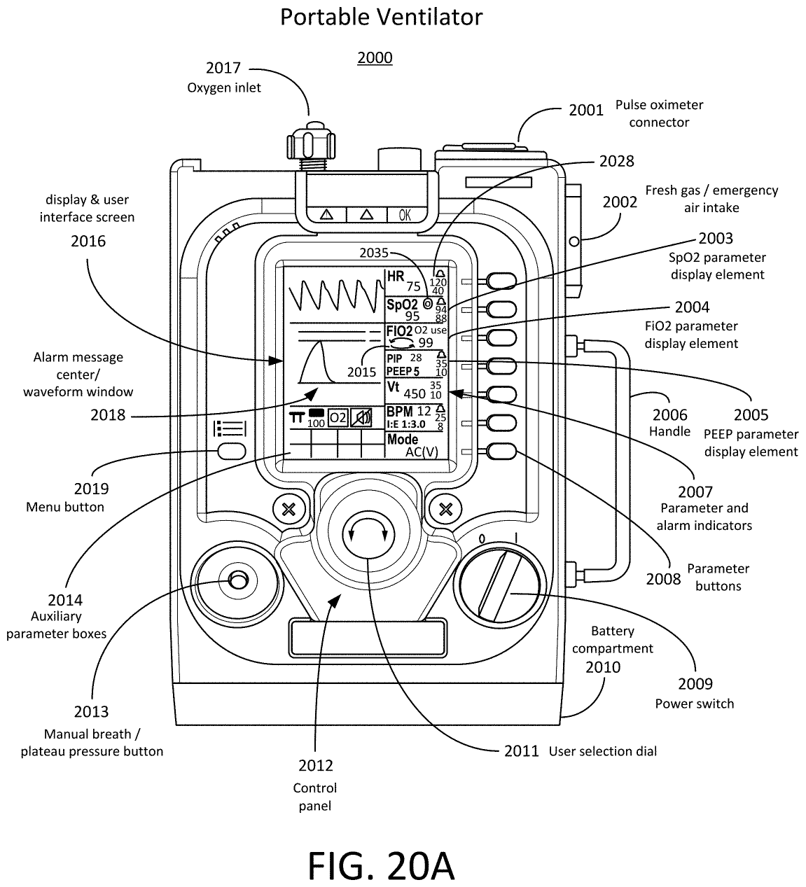

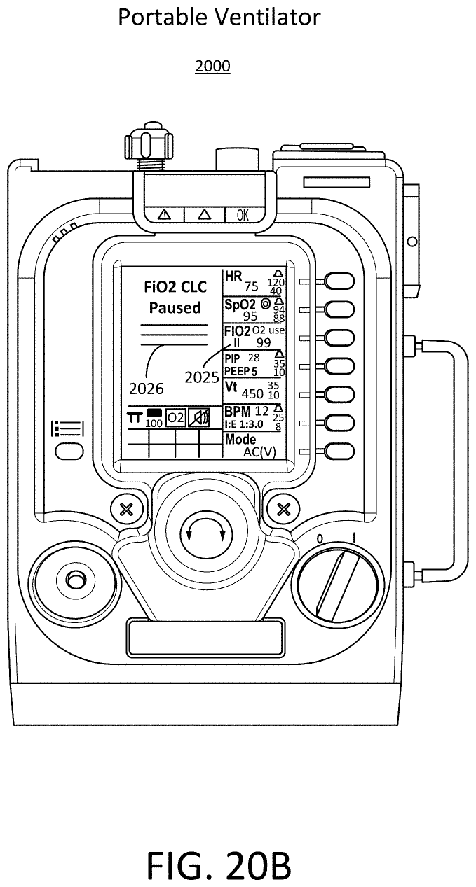

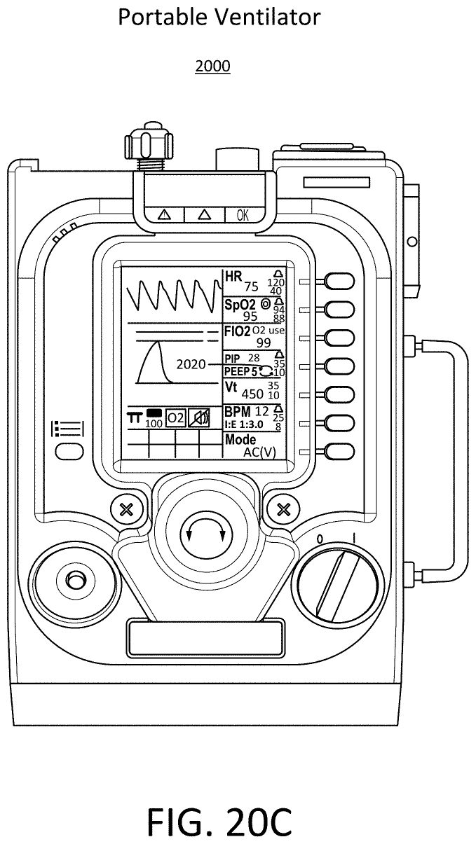

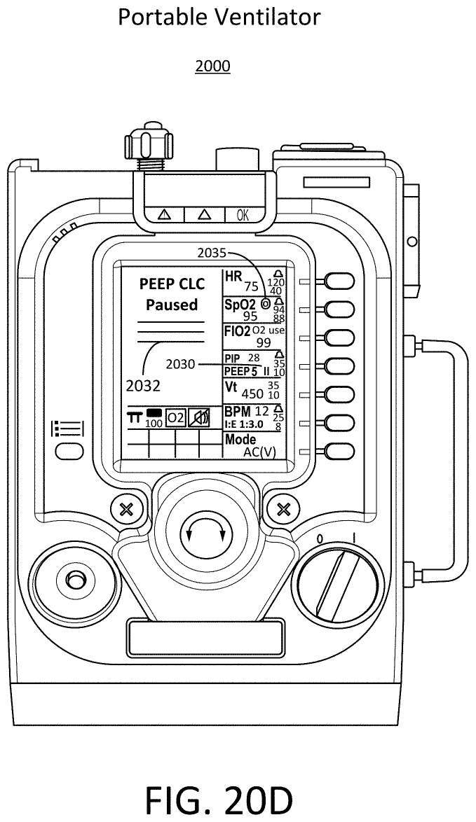

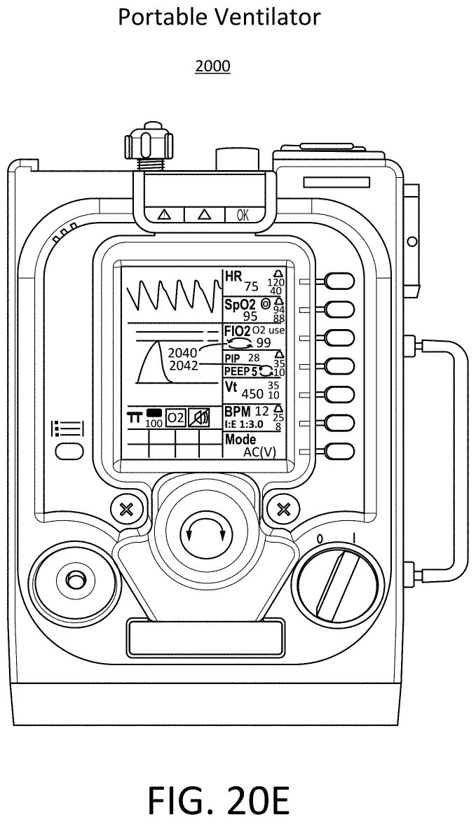

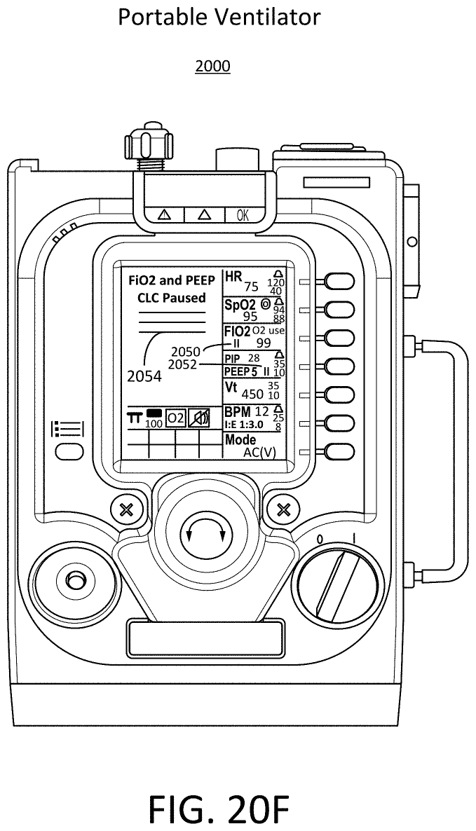

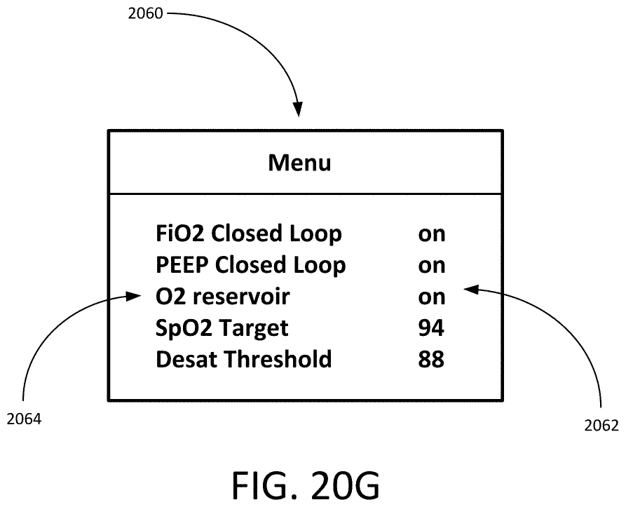

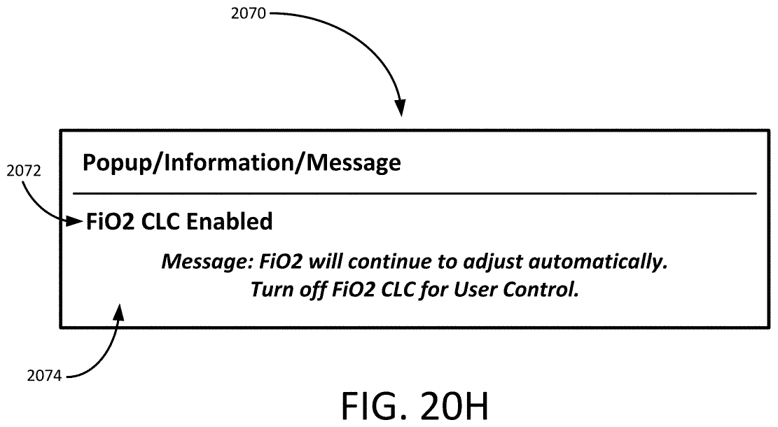

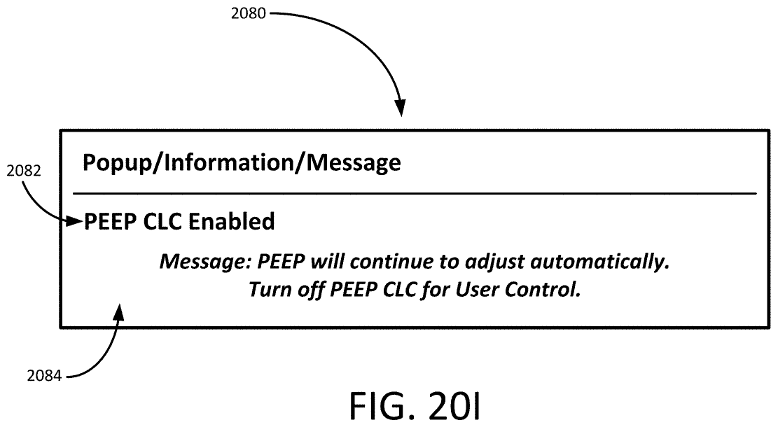

If, at step 356, it is determined that, according to the PEEP selection rules, a PEEP decrease is recommended, then, at step 360, the user is prompted (such as via a graphical user interface of a display of a portable ventilator 2000 as described with reference to FIGS. 20A-H) to accept the recommended new, decreased PEEP. If the user accepts, then, at step 364, PEEP is decreased accordingly (such as, for example, via appropriate actuation of an exhalation control valve 2114, as depicted in FIG. 21). If the user does not accept, then PEEP is not changed. It is noted that, in other embodiments, user acceptance of a PEEP change is not required and the check can be performed automatically without requiring user intervention or confirmation.

FIG. 7A is flow diagram illustrating aspects of an example method 700 of operation of a PEEP control engine 701 of a ventilation system incorporating closed loop control, in accordance with embodiments of the present disclosure. As depicted, PEEP control engine 701 is in coordination with central control 702.

At step 704, the method 700 queries, has there has been no FiO2 change (or, in some embodiments, no FiO2 change beyond a minimum threshold), such as, e.g., 1%, or a fraction of 1%, or 1-2%) in a last certain period of time, which may be 10 minutes (however, in various embodiments, the period may be, e.g., 5-30 minutes) or patient desaturation condition, such as, for example, a patient SpO2 at or below 88% (however, in various embodiments, the threshold may be, e.g., 85%-90%) that currently exists and has lasted for at least another last certain set period of time, such as 2 minutes (however, in various embodiments, the period may be, e.g., 1-3 minutes)? If the answer is “no,” then PEEP is not currently eligible for change. However, if the answer is “yes,” then PEEP may be eligible for change the method 700 proceeds to step 706.

At step 706, the PEEP selection rules are utilized to determine whether a PEEP increase, PEEP decrease, or no change in PEEP is indicated.

At step 706, if no PEEP change is indicated, then PEEP is not changed.

At step 706, if a PEEP increase is indicated by the PEEP selection rules, then, at step 714, the method 700 queries whether there has been a PEEP change within a particular threshold past period of time for a PEEP increase, such as, for example, in the last 10 minutes or in the last 20 minutes (however, in various embodiments, the period may be, e.g., 5-45 minutes). If there has been a PEEP change during the threshold past period of time for a PEEP increase, then PEEP is not eligible for change. If there has not been a PEEP change during the threshold past period of time for a PEEP increase, x then, at step 716 the method 700 queries whether the patient's hemodynamics are sufficient, or evidenced or determined to be sufficient (such as via use of a blood pressure sensor or monitor 118, as depicted in FIG. 1, coupled with the patient). If the patient's hemodynamics are determined not to be sufficient (e.g., which may be indicated by a low systolic blood pressure), then PEEP is not eligible for potential change. In some embodiments, for example, if the patient's hemodynamics are determined not to be sufficient (e.g., as indicated by a high enough systolic blood pressure, for example), then PEEP may not be eligible for potential change for at least a particular predetermined period of time, such as 30 minutes (or, e.g., between 15-45 minutes).

If, however, the patient's hemodynamics are determined to be sufficient (e.g., as indicated by a high enough systolic blood pressure, for example, such as may be measured, for example, using the blood pressure sensor or monitor, as depicted in FIG. 1), then, in some embodiments, at step 710, a user of the ventilation system may be prompted via a graphical user interface to accept an indicated increase in PEEP. At step 710, if the user accepts the indicated increase in PEEP, then, at step 712, PEEP is increased according to the new setting indicated by the PEEP selection rules (such as, for example, via appropriate actuation of an exhalation valve 2114, as depicted in FIG. 21). Furthermore, in some embodiments, a later, additional user confirmation of the PEEP increase is required, such as 10 minutes after the PEEP increase (or, e.g., 5-20 minutes). It is noted that, in some embodiments, user acceptance may not be required, in which case step 710 would be omitted. In some embodiments, however, no user acceptance may be required. For example, the controller may compare the current or most recent systolic blood pressure with a threshold systolic blood pressure value, and allow the PEEP increase if the threshold is met.

If, at step 706, a PEEP decrease is indicated by the PEEP selection rules, then, at step 708, the method 700 queries whether there has been a PEEP change within a particular threshold period of time for a PEEP decrease, such as, for example, in the last hour (however, in various embodiments, the period may be, e.g., 30-120 minutes). If there has been a PEEP decrease during the threshold past period of time for PEEP decrease, then PEEP is not eligible for change. If there has not been a PEEP change during the threshold past period of time, then, at step 710, if the user accepts the indicated decrease in PEEP, then, at step 712, PEEP is decreased (such as via appropriate actuation of the exhalation valve 2114, as depicted in FIG. 21) according to the new setting indicated by the PEEP selection rules. It is noted that, in some embodiments, user acceptance may not be required, in which case step 710 would be omitted.

FIG. 7B illustrates a method 750 that is, in many regards, may be similar to the method 700 of FIG. 7A. However, differences include that boxes 714 and 708 of FIG. 7A have been replaced in FIG. 7B by boxes 754 and 758. Also, box 704 of FIG. 7A is not included in FIG. 7B. Other boxes and steps may be similar between the two methods. Steps 754 and 758 are described as follows.

Step 754 of FIG. 7B replaces step 714 of FIG. 7A. At step 754, the method 750 queries, (A) has there not been a PEEP change within a threshold past period of time for a PEEP increase; and (B) have at least one of the following two conditions been met: (1) has FiO2 either not changed (or, in some embodiments, not changed beyond a minimum threshold, such as, e.g., 1%, or a fraction of 1%, or 1-2%), or continuously increased, in the last set period (e.g., 10 mins), such as by increasing at every check; (2) has there been a desat (e.g., SpO2 at or below 88%) that has lasted for at least a last set period (e.g., 2 minutes)? If the answer is “no,” then PEEP is not eligible for change. If the answer is “yes,” then the method 750 proceeds to step 716.

Step 758 of FIG. 7B replaces step 708 of FIG. 7A. At step 758, the method 750 queries, has there not been a PEEP change within a particular threshold past period of time for a PEEP decrease, such as, for example, in the last 10 minutes or in the last 20 minutes (however, in various embodiments, the period may be, e.g., 5-45 minutes), and has FiO2 either not changed (or, in some embodiments, not changed beyond a minimum threshold, such as, e.g., 1%, or a fraction of 1%, or 1-2%) or continuously decreased (such as by decreasing at every check), in a last certain period of time, which may be 10 minutes (however, in various embodiments, the period may be, e.g., 5-30 minutes)? If the answer is “no,” then PEEP is not eligible for change. If the answer is “yes,” then the method 750 proceeds to step 710.

As discussed above, whereas the exemplary method 700 of FIG. 7A provides that PEEP change occur when FiO2 has remained steady over a set period of time, the exemplary method 750 of FIG. 7B recognizes that an increase or decrease of FiO2 could also serve as an input as to whether PEEP should increase or decrease. In particular, as noted herein, an observed increase in FiO2 over a set period may indicate that the patient's health status is deteriorating and the patient may be in need of increasing PEEP support; therefore, increase of PEEP may be appropriate, and decrease of PEEP may be less appropriate or inappropriate. Conversely, an observed decrease in FiO2 over a set period may indicate that the patient's health status is improving and the patient may not be in need of the added PEEP support.

FIG. 8 is a block diagram illustrating aspects of example PEEP selection rules 800, in accordance with embodiments of the present disclosure. In some embodiments, PEEP selection rules, such as those depicted in FIG. 8, are used, such as by or in a PEEP control engine of a ventilation system, to determine whether a PEEP setting change is indicated. However, in some embodiments, even if a PEEP setting change would be or is indicated by the PEEP selection rules, PEEP is not in fact changed if it is determined that PEEP is not eligible for the change. FIGS. 6 and 7 provide examples of conditions that may be required for PEEP change eligibility, in some embodiments.

In some embodiments, during ventilation of a patient, a PEEP setting change is indicated by the PEEP selection rules only if the FiO2 setting changes so as to fall outside of an FiO2 range associated with the current PEEP setting (such as above the relevant range, as indicated by thick lines, such as line 806, or below the relevant range, as indicated by thin lines, such as line 804). Moreover, in some embodiments, each of a number of possible PEEP settings is associated with a particular FiO2 range, but adjacent FiO2 ranges may overlap. As a result, the current PEEP setting is indicated as maintained unless the FiO2 setting changes not only (a) enough to fall into a portion of the current FiO2 range that overlaps with another FiO2 range associated with a different PEEP setting, but (b) enough to go beyond the overlapping portion as well as into an FiO2 range associated with a different PEEP setting.

In some embodiments, this can be viewed as creating an intended tendency in favor of maintaining the current PEEP setting. For instance, the PEEP selection rules may be structured so as to exhibit what may be viewed as a hysteresis effect, in that whether a PEEP change is currently indicated is affected by the PEEP level in use immediately prior to the determined potential new PEEP level. In some embodiments, this, in turn, can improve patient safety, for example, given that, while PEEP setting changes are necessary under appropriate circumstances, frequent or rapidly changing PEEP settings can cause serious risk to the patient.

Furthermore, in some embodiments, possible or allowed PEEP settings are determined to include a discrete set of particular PEEP settings, or levels, each of which is associated with an FiO2 range, where FiO2 ranges corresponding to neighboring PEEP settings are overlapping. In some embodiments, a PEEP setting can only be adjusted at most so as to change by one level up or down, even if, for example, the FiO2 setting changes dramatically. In some embodiments, this can improve patient safety by not allowing large, immediate jumps in the PEEP setting.

In the example shown in FIG. 8, PEEP can only be set according to one of a discrete set of PEEP levels—as shown, those levels are 5, 7, 9, 11, 13 and 15 cm H2O (however, in various embodiments, the number of levels, setting of each level, and maximum and minimum levels, may be different, such as, e.g., differing by less than, in cm H2O, 0-0.25, 0.25-0.5, 0.5-0.75, 0.75-1, 1-1.25, 1.25-1.5, 1.5-1.75, 1.75-2, 2-2.25, 2.25-2.5, 2.5-2.75, 2.75-3, 3-3.5 or 3.5-4. Furthermore the minimum PEEP setting may be different than 5 cm H2O, such as, e.g., in cm H2O, 0-1, 1-2, 2-3, 3-4, 4-5, 5-6, 6-7, 7-8, 8-9 or 9-10. Furthermore the maximum PEEP setting may be different than 15 cm H2O, such as, e.g., in cm H2O, 10-11, 11-12, 12-13, 13-14, 14-15, 15-16, 16-17, 17-18, 18-19, 19-20, 20-21, 21-22, 22-23, 23-24 or 24-25. As depicted, the boxes 802 represent particular FiO2 ranges corresponding with particular PEEP levels. Specifically, PEEP=5 cm H2O corresponds with FiO2 range 21-39%; PEEP=7 cm H2O corresponds with FiO2 range 30-49%; PEEP=9 cm H2O corresponds with FiO2 range 40-59%; PEEP=11 cm H2O corresponds with FiO2 range 50-69%; PEEP=13 cm H2O corresponds with FiO2 range 60-79%; and, PEEP=15 cm H2O corresponds with FiO2 range 70-100%. (however, in various embodiments, the FiO2 ranges associated with particular PEEP settings or levels may be different).

As can be seen, although each PEEP level corresponds with a particular FiO2 range, adjacent FiO2 ranges overlap. For example, the FiO2 range corresponding with PEEP=7 cm H2O, which is FiO2 of 30-49%, overlaps with the FiO2 range corresponding with PEEP=9 cm H2O, which is FiO2 of 40-59%—specifically, the ranges overlap for FiO2 of 40-49%.

As a specific illustration of operation of the PEEP selection rules, for example, during ventilation of a patient, the current PEEP setting is 9 cm H2O, which has a corresponding FiO2 range of 40-59%. The system, such as a ventilation control engine of a ventilation system, determines the current FiO2 setting.

If the current FiO2 setting is, for example, 45%, then no PEEP change is indicated, since 45% falls within the FiO2 range of 40-59%. This is the case even though 45% also falls within the FiO2 range of 30-49% associated with the next lower PEEP level of 7 cm H2O. Even though the current FiO2 of 45% falls in the overlapping portion of the two ranges, since it does not fall outside of the range corresponding with the current PEEP level, no PEEP change is indicated.

Similarly, if the current FiO2 setting is, for example, 55%, still no PEEP change is indicated. Even though 55% falls within the overlapping portion of the FiO2 ranges corresponding with PEEP=7 cm H2O and PEEP=9 cm H2O, since it falls within the FiO2 range of 40-59% that corresponds with the current PEEP setting of 7 cm H2O, no PEEP change is indicated.

However, if the current FiO2 setting is, for example, 38%, then a PEEP decrease is indicated. An FiO2 of 38% falls below the range of 40-59% corresponding with the current PEEP level of 9 cm H2O, and into the FiO2 range of 30-49% corresponding with a PEEP of 7 cm H2O. In this instance, a PEEP decrease to the next lower level of 7 is indicated by the PEEP selection rules, as suggested by the downward pointing arrow at the left of the box associated with PEEP=9 cm H2O. It is to be understood that actual FiO2 changes, such as from second to second, may be, for example, much less than stated in these examples, and the numbers provided in the examples are merely for illustrative purposes.

Similarly, if the current FiO2 setting is, for example, 61%, then a PEEP increase is indicated. An FiO2 of 61% falls above the range of 40-59% corresponding with the current PEEP level of 9 cm H2O, and into the FiO2 range of 50-69% corresponding with a PEEP of 11 cm H2O. In this instance, a PEEP increase to the next higher level of 11 is indicated by the PEEP selection rules, as suggested by the upward pointing arrow at the right of the box associated with PEEP=9 cm H2O.

Assume that the FiO2 is 61% and the PEEP is in fact increased to PEEP=11 cm H2O (which may assume that PEEP is eligible for the increase), then, at the next check/cycle/time period, the FiO2 range of 50-69%, which corresponds with PEEP=11 cm H2O, is referenced. A PEEP decrease or increase will be indicated only if the PEEP changes so as to fall below or above the range of 50-69%.

In some embodiments, even if FiO2 changes drastically, so as to, for example, fall into an FiO2 range associated with a PEEP level more than one level lower or higher than the current PEEP level, the PEEP will only be indicated as decreased or increased by one level. For example, assume that the current PEEP is 9 cm H2O and the FiO2 is very low (as if it just dropped dramatically), such as 25%. Even though an FiO2 of 25% falls into the FiO2 range corresponding with a PEEP of 5 cm H2O, the PEEP selection rules will indicate a PEEP decrease to one level lower than the current PEEP level. That is, the PEEP selection rules will indicate that the PEEP be decreased to 7 cm H2O, even though the current FiO2 actually falls below the range FiO2 range of 30-49% that corresponds with a PEEP of 7 cm H2O. Similarly, if FiO2 is 100% (as if it just increased dramatically), a PEEP increase would be indicated to a PEEP of 11 cm H2O, even though the FiO2 of 100% falls beyond the FiO2 range that corresponds with PEEP of 11 cm H2O and into the FiO2 range that corresponds with the maximum PEEP of 15 cm H2O, which is several levels higher than the current PEEP of 9 cm H2O.

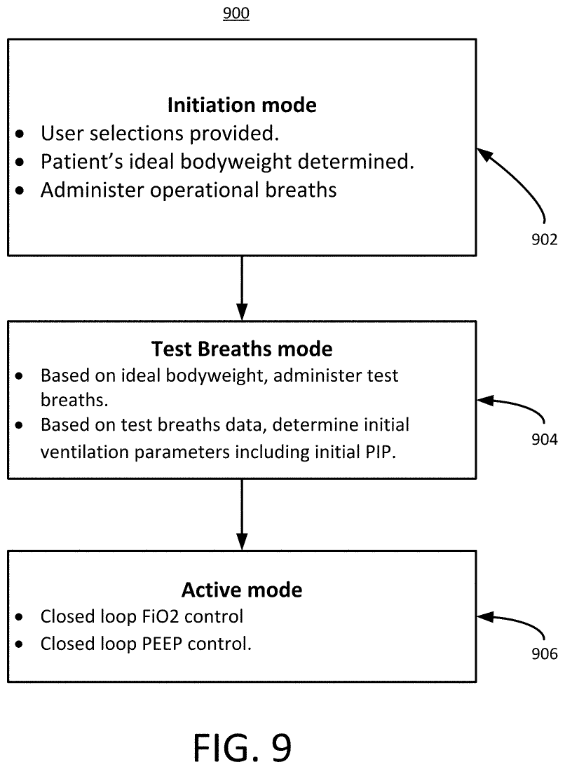

FIG. 9 is a flow diagram illustrating an example multi-mode method 900 of operation of a ventilation system incorporating closed loop control, in accordance with embodiments of the present disclosure. The method 900 includes an initiation mode 902, followed by a test breaths mode 904, followed by an active mode 906.

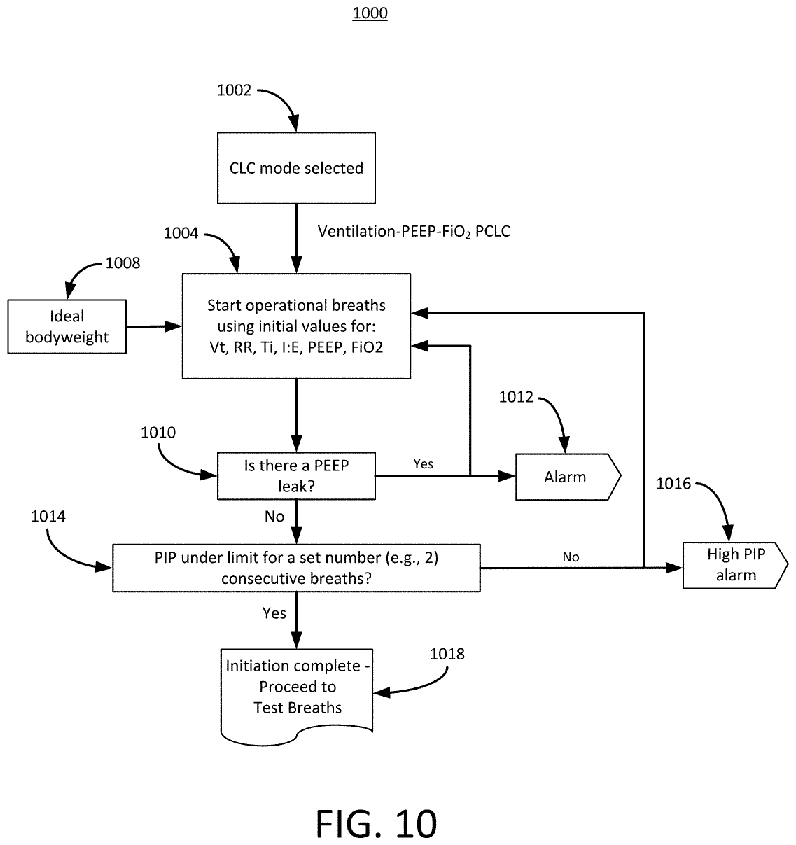

During the initiation mode 902, one or more user selections may be made, such as selection of PRVC ventilation, although in some embodiments, no such user selection is provided and the type of ventilation is set according to a pre-configured default. A user of the ventilator may be prompted to provide the patient's gender and height, and the patient's PBW may be determined. Based on the patient's PBW, one or more ventilation parameters such as the tidal volume to be delivered per breath may be determined. Furthermore, one or several operational breaths are administered to the patient such as, for example, via a gas delivery apparatus 106 and using a facemask 110, as depicted in FIG. 1, coupled with the patient, or intubation. Further details relating to an initiation mode are provided with reference to FIG. 10.

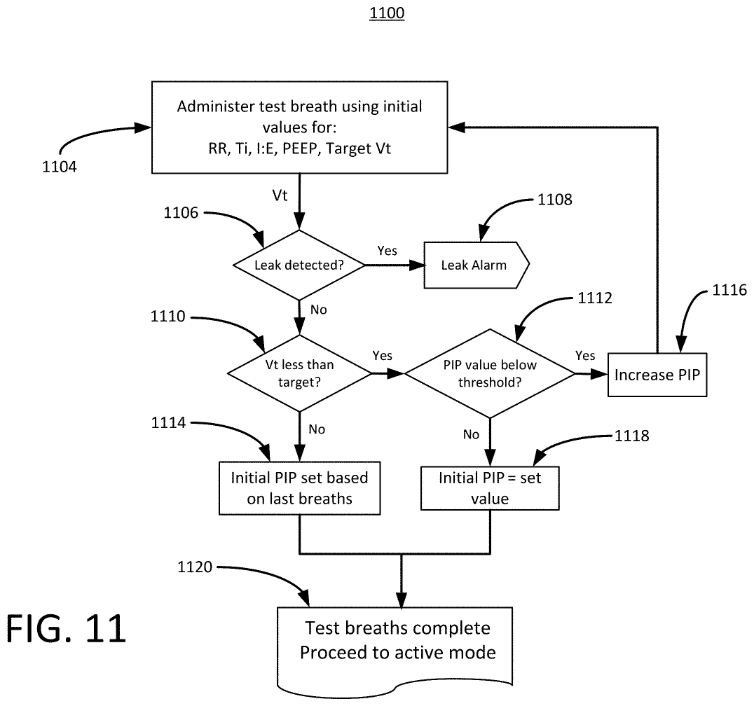

During the test breaths mode 904, one or several breaths may be delivered to the patient such as, for example, via a gas delivery apparatus 106 and using a facemask 110, as depicted in FIG. 1, coupled with the patient, or intubation, using certain ventilation parameters that are determined based at least in part on the patient's gender and PBW. Based on data associated with the breaths, a set of initial active mode ventilation parameters are determined, including an initial PIP (PIP(0)) setting. Further details relating to a test breaths mode are provided with reference to FIG. 11.

During active mode 906, ventilation is provided to the patient such as, for example, via a gas delivery apparatus 106 and using a facemask 110, as depicted in FIG. 1, coupled with the patient, or intubation, which may include closed loop control of FiO2 and PEEP, as detailed in embodiments described herein.

FIG. 10 is a flow diagram illustrating aspects of a method 1000 of operation of an example initiation mode of a ventilation system incorporating closed loop control, in accordance with embodiments of the present disclosure. The purpose of this initiation period may be to ensure that the ventilator is able to operate under normal parameters and limits.

At step 1002, a user selects CLC mode. However, in some embodiments, no such user selection is required. At step 1004, operational breaths are administered, such as, for example, via a gas delivery apparatus 106 and using a facemask 110, as depicted in FIG. 1, coupled with the patient, or intubation, using initial values for parameters including Vt, RR, I:E, PEEP and FiO2.

At step 1010, the method 1000 queries whether there is a PEEP leak, such as, for example, by determining whether measured proximal airway pressure is steady at the end of expiration. This may be done, for example, by determining that airway pressure has not decreased by more than a particular threshold amount or percentage at the end of an expiration. If measured proximal airway pressure is not steady at the end of expiration, then, at step 1012, an alarm, such as a PEEP leak alarm, is triggered, and the method 1000 returns to step 1004. If measured proximal airway pressure is held steady at the end of expiration, then, at step 1014, the method 1000 queries whether PIP is under limit, such as under 35 cm H2O (or, e.g., between 30-40 cm H2O), for a certain number of consecutive operational breaths, such as 2 (however, in various embodiments, the number of breaths may be, e.g., 1-10). If not, then, at step 1012, a high PIP alarm is triggered. If so, then initiation mode is complete and, at step 1018, the method 1000 proceeds to test breaths mode.

FIG. 11 is a flow diagram illustrating aspects of a method 1100 of operation of an example test breaths mode of a ventilation system incorporating closed loop control, in accordance with embodiments of the present disclosure. In some embodiments, in test breaths mode, the system may deliver a series of breaths, and based on associated collected data, determine an initial PIP(0) setting to be used in active mode, as well as ensure that the system is leak-free.

At step 1104, breaths are administered, such as, for example, via a gas delivery apparatus 106 and using a facemask 110, as depicted in FIG. 1, coupled with the patient, or intubation, using initial values for RR, Ti, I:E, PEEP and target Vt. In some embodiments, the target Vt may be determined based on the patient's predicted body weight, as input from the patient's height and gender. At step 1106, the method 1100 queries whether any leaks are detected, such as, for example, by determining if the measured airway pressure is decreasing at the end of expiration or by determining whether a detected amount of flow is not equal to, or not close enough to, the amount that is set to be initially provided by the ventilator. If so, then, at step 1108 a leak alarm is triggered. In some embodiments, if the leak alarm is triggered, the system may continue delivering ventilation with current settings until further use action is taken.

If not, then, at step 1110, the method 1100 queries whether the delivered Vt is less than the target Vt.

At step 1110, if Vt is not less than the target Vt, then, at step 1114, PIP(0) is set based on one or more last breaths. For example, in some embodiments, PIP(0) is calculated by interpolation between the current and previous PIP settings, but, in various embodiments, different models or calculation methods may be used. At step 1120, test breaths mode is complete and active mode can be entered.

If Vt is less than the target Vt, then, at step 1112, the method 1100 queries whether PIP is below threshold. If so, then, at step 1116, PIP is increased, and the method 1100 proceeds back to step 1104. If not, then, at step 1118, PIP(0) is set to a particular value. At step 1120, test breaths mode is complete and active mode can be entered.

In some embodiments, the first breath will have a PIP of 10 cm H2O (however, in various embodiments, the first breath may have a PIP of, e.g., 5-15 cm H2O), which may increase by 5 cm H2O (however, in various embodiments, the increase may be, e.g., 3-7 cm H2O) for each subsequent breath up to a maximum of 30 cm H2O (however, in various embodiments, the maximum may be, e.g., 25-35 cm H2O). Once the delivered volume is greater than or equal to the target volume, the breaths are stopped and initial PIP is calculated by interpolation between the current and previous PIPs. If a PIP of 30 cm H₂O does not achieve the desired target volume, the initial PIP(0) is set to 30 cm H₂O (however, in various embodiments, the PIP(0) may be set to, e.g., 25-35 cm H2O).

FIG. 12A is a flow diagram illustrating an example method 1200 of operation of a gain related algorithm, in accordance with embodiments of the present disclosure, which may, for example, be implemented by a ventilation system as disclosed in embodiments herein.

In particular, FIG. 12A provides an example of use of a proportional gain term and a derivative gain term in determining an overall correction that is applied in adjusting FiO2 based on SpO2. An overview is provided, followed by a detailed example.

In some embodiments, at a given time, the overall adjustment to the FiO2 may be based on a correction that is calculated as the sum of a proportional gain term and a derivative gain term. A positive overall correction value may indicate an increase in FiO2, while a negative overall correction value may indicate a decrease in FiO2. The proportional gain term may operate to affect the FiO2 adjustment to create a tendency to draw the (current) SpO2 toward a Target SpO2. Therefore, if SpO2 is below the Target SpO2, it will tend to increase FiO2 to draw SpO2 up, but if SpO2 is above the Target SpO2, it will tend to draw FiO2 down to draw SpO2 down. Furthermore, the magnitude of the proportional gain term may be proportional to the distance, or value difference, of the SpO2 from the Target SpO2. As such, the further the current SpO2 is from the Target SpO2, the greater the proportional gain term will be. Still further, in some embodiments, if SpO2 is outside of a certain range that includes the target SpO2, then the proportional gain term will be greater. Overall, the proportional gain term may operate to create a tendency to affect the FiO2 adjustment so as to draw the SpO2 toward the Target SpO2, and the magnitude of the proportional gain term may be greater when the SpO2 is further from the Target SpO2. The proportional gain term may be unaffected by whether SpO2 is increasing or decreasing, and based only on the current SpO2.

The derivative gain term may operate as follows. When SpO2 is evidenced as decreasing (as may be indicated by the current SpO2 as compared with the last SpO2, or an average of a set of most recent SpO2 measurements compared to an average of a set of SpO2 measurements prior to that, for example, or in other ways), the derivative gain term may operate to create a tendency to draw the FiO2 up to draw SpO2 up. Furthermore, the magnitude of the derivative gain term may effectively be proportional to the effective rate of decrease of the SpO2, in that it may be proportional to a difference between a current SpO2 (or moving average of most recent SpO2, for example) and a last SpO2 (or a moving average of SpO2 prior to the most recent SpO2, for example). However, when SpO2 is increasing, the derivative gain term may be 0, creating no tendency to draw the FiO2 down to draw the SpO2 down. In this sense, the derivative gain term may be considered to have an asymmetric effect on FiO2 change, tending to increase FiO2 when SpO2 is decreasing, but having no symmetric effect of tending to decrease the FiO2 when SpO2 is increasing. The derivative gain term may be unaffected by the current SpO2, as such, and based only on whether SpO2 is increasing or decreasing, and, if decreasing, at what effective rate.

Therefore, in some embodiments, the proportional gain term may operate to affect the FiO2 adjustment to create a tendency to draw SpO2 toward the Target SpO2, in proportion to how far the SpO2 is from the Target SpO2. The derivative gain term may operate to affect the FiO2 adjustment to increase the FiO2 in proportion to an effective rate of decrease of the SpO2, but have no effect when SpO2 is increasing. Overall, whether positive or negative, the proportional gain term will be greatest when the (current) SpO2 is farthest from the Target SpO2. If SpO2 is increasing, the derivative gain term will be zero, but if SpO2 is decreasing, the derivative gain term will be positive and in proportion to the effective rate of decrease of the SpO2. The proportional gain may, in some embodiments, use a moving average of current or recent SpO2, for example, or other calculation models or methods.

As such, if SpO2 is increasing, the adjustment may be affected only by the proportional gain term. However, if SpO2 is decreasing, the adjustment may be affected by both the proportional gain term and the derivative gain term.

If SpO2 is far above target and decreasing slowly, then the proportional gain term in the calculated FiO2 correction may be negative (tending to draw FiO2 down) and the derivative gain term may be positive (tending to draw FiO2 up), but the proportional gain term may be greater in magnitude than the derivative gain term. In such an instance, the overall correction may be negative (so that FiO2 is decreased) due to the proportional gain term, but the derivative gain term may effectively operate as a partial brake on, or to mitigate the magnitude of, the overall decrease.

However, if SpO2 is slightly above target but decreasing rapidly, then the proportional gain term may be negative and the derivative gain term may be positive, but the proportional gain term may be lesser in magnitude than the derivative gain term. In such an instance, the overall FiO2 adjustment may be positive due to the derivative gain term, but the proportional gain term may effectively operate as a partial brake on, or to mitigate the magnitude of, the overall increase.

As such, in some embodiments, when SpO2 is increasing, the FiO2 correction may be entirely due to the proportional gain term, and FiO2 will change so as to draw the SpO2 towards the Target SpO2. However, when the SpO2 is decreasing, the proportional gain term and the derivative gain term may both operate to affect the FiO2 adjustment, whether in an additive or opposite fashion.

It is notable that, in a rapidly decreasing situation, where, for example, patient SpO2 is much lower than target and decreasing rapidly, both the proportional gain term and the derivative gain term will be large and positive, leading to the FiO2 being adjusted by a large increase. In this instance, the proportional gain term and the derivative gain term both contribute to an appropriate rapid increase in FiO2 in response to what may be an urgent actual or approaching patient desaturation crisis.

In FIG. 12A, at step 1202, the system waits for a predetermined time increment, such as one second, (however, in some embodiments, the wait lop time increment may be different, e.g., a fraction of a second, between 1-2 seconds, multiple seconds, more than one minute, an irregular or varying interval, etc.). At step 1204, patient SpO2 (current SpO2) is determined. For example, an oximetry sensor, coupled with the patient, may be used to obtain signals representation of the patient's SpO2, and a controller may receive the signals and determine the patient's SpO2 based on the signals. At step 1206, the system confirms that the determined SpO2 is within a valid range, such as by confirming that SpO2 is between 0% and 100%. In some embodiments, more than just a single measured SpO2 may be used, such as, for example, an SpO2 moving average, a noise filtered SpO2 value, etc.

At step 1208, the system calculates Derivative Error (DE) as follows.

Derivative Error (DE)=LastError−Error (Equation 1)

Where:

Error=Average of (Target SpO2−SpO2) for N most recent times/cycles

LastError=Average of (Target SpO2−SpO2) N next most recent times/cycles

Conceptually, for a constant Target SpO2, DE will be negative (which will produce a positive Derivative Gain term, as explained below), when Error is greater than LastError—that is, when SpO2 is, overall, decreasing. Conversely, DE will be positive (which will produce a Derivative Gain term of 0, as explained below), when LastError is greater than Error—that is, when SpO2 is, overall, increasing.

In some embodiments, moving averages are utilized, such as to take into account different SpO2 over different amounts of time or samples, or to effectively filter out random or unpredictable variation or noise, or to prevent undesirably rapidly varying or choppy FiO2 or SpO2 response, for example. However, in some embodiments, other methods are used, including methods that use only the current and last SpO2, or that use a different technique in determining a rate or effective of increase or decrease in SpO2, or that use a different technique in filtering or smoothing, for example.

Particularly, in the embodiment depicted, Error is calculated by determining an average of (Target SpO2−SpO2) for measurements for N most recent times, and LastError is calculated by determining an average of (Target SpO2−SpO2) for N next most recent times. A simplified example follows.

Assume that Target SpO2 is 94%, the system loop time increment is 1 second, N=4, and SpO2 has been measured over the last 7 seconds as follows:

TABLE 1
(Measured SpO2 values not actual-actual
changes may be much smaller)

| | Target | Measured | |
|---|---|---|---|
| Time (in seconds) | SpO2 | SpO2 | Error |
| 0 (Current time) | .94 | .91 | .03 |
| 1 (1 second ago) | .94 | .92 | .02 |
| 2 | .94 | .92 | .02 |
| 3 | .94 | .91 | .03 |
| 4 | .94 | .93 | .01 |
| 5 | .94 | .92 | .02 |
| 6 | .94 | .92 | .02 |
| 7 | .94 | .93 | .01 |

LastError is calculated as the average of Target SpO2−Measured SpO2 for each of times 4−7=(0.01+0.02+0.02+0.01)/4=0.06/4=0.015. Error is calculated as the average of Target SpO2−Measured SpO2 for each of times 0−3=(0.03+0.02+0.02+0.03)/4=0.10/4=0.025 (Error=0.025). DE is then calculated as LastError−Error=0.015−0.025=−0.01 (DE=−0.01). The negative DE will lead to a positive derivative gain term, as calculated below. This is appropriate since, overall, SpO2 is decreasing. If SpO2 had been, overall, increasing, DE would have been positive, leading to a zero derivative gain term, which is also appropriate. It is also noted that, if the SpO2 was even lower on average for the more recent times, 0-3 seconds, then DE would still be negative but would have a larger magnitude, which would lead to an even greater derivative gain term, below.

At step 1210, the system determines whether SpO2 is within a particular set range, such as between 93%-99% (however, in various embodiments, a different range may be used, such as between 90%-100%). If SpO2 is within the set range, then a proportional gain term, K, is set to a smaller value (e.g., the value G), whereas, if SpO2 is outside of the set range (e.g., less than 93% or greater than 99%), then K is set to a larger value, such as 2G (or, e.g., between G and 2G, or between 2G and 3G). The proportional gain term can lead to a much more rapid FiO2 response when patient SpO2 is far from a target value such as 94%, which can include a much more rapid FiO2 increase when patient SpO2 is very low. In some embodiments, however, proportional gain is not varied based on any threshold or range.

In some embodiments, G may be, for example, 0.0025 (or, e.g., between 0.001 and 0.015, such as between 0.001 and 0.005, between 0.005 and 0.010, or between 0.010 and 0.015), and 2G may be, for example, 0.005, (or, e.g., between 0.002 and 0.03, such as between 0.002 and 0.01, between 0.01 and 0.02, or between 0.02 and 0.03).

Using the above example, current SpO2 is 0.90, which is outside of the range of 93%-99%. Therefore, K would be the larger value 2G, so K=0.005.

At step 1212, if DE is equal to or less than 0, then derivative gain (DG) is set to 0, whereas, if DE is greater than 0, then DG is set to a set number, which may be a negative fraction.

In some embodiments, DG may be, for example, −0.03 (or, e.g., between 0 and −0.3, such as between 0 and −0.1, between −0.1 and −0.2, or between −0.2 and −0.3).

At step 1214, an FiO2 correction (FC) is calculated as follows.

FiO2 Correction (FC)=(Error*K)+(DE*DG) (Equation 3)

Where (Error*K) is the proportional gain term, and (DE*DG) is the derivative gain term.

In this example, the proportional gain term (Error*K) would be calculated as (0.025)(0.005)=0.000125.

Using the above example, the derivative gain term (DE*DG) would be calculated as (−0.01)(−0.03)=0.0003, which is a positive derivative gain term.

Therefore, both derivative and proportional gain will tend to increase FiO2 in this example, which is appropriate since SpO2 is decreasing (leading to a positive derivative gain term) and below the Target SpO2 (leading to a positive proportional gain term).

Overall, FiO2 Correction, or FC would be calculated as FC=(Error*K)+(DE*DG)=(0.025) (0.005)+(−0.01)(−0.03)=0.000125+0.0003=0.000425 (a positive correction value, or FiO2 increase).

At step 1216, an alert or warning is displayed if the FiO2 is adjusted by at least a set percentage, such as 10% (or, e.g., 5%-20%) in the last set period of time, such as 10 minutes (or, e.g., 5-30 minutes).

At step 1218, an updated FiO2 setting (“updated FiO2”) is calculated as follows.

Updated FiO2=current FiO2+FiO2 correction (FC) (Equation 4)

Using the above example, if current FiO2 is, for example, 0.5, then Updated FiO2 would be calculated as 0.5+FC=0.5+0.000425=0.500425.

The FiO2 setting is then adjusted by being increased to the calculated Updated FiO2, such as, for example, via appropriate actuation of an oxygen source valve 2106, as depicted in FIG. 21. Generally, such adjusting can include, as appropriate, the FiO2 setting being increased, decreased, or kept the same if the FiO2 correction (FC) is equal to 0 so that the updated FiO2 is the same as the current FiO2.

The data depicted in FIGS. 12B-12E, FIG. 18 and FIGS. 19A-19B all relate to the same animal (porcine) study results.

FIGS. 12B-12E provide graphical illustrations 1238, 1240, 1270, 1295 of an example of a set of animal (porcine) study results tracking FiO2 setting relative to SpO2, in accordance with embodiments of the present disclosure.

FIG. 12B shows, over minutes 0-140, plots of porcine SpO2 1226, target SpO2 1223, FiO2 1220, and FiO2 correction 1236, including the proportional gain term 1232 and the derivative gain term 1234. Between approximately minutes four and eleven (as also shown in FIG. 12C), it can be seen that SpO2 1230 is below the target SpO2, leading to a large proportional gain term and an overall large correction, which in turn leads to an increasing FiO2 1222. At approximately time 56 minutes, a spike 1233 can be seen in the derivative gain term 1234, resulting from a rapid decrease in SpO2 1227 at that time. Generally, from approximately time 60 minutes, SpO2 is above the target SpO2, leading to a negative overall correction factor and a consequently decreasing FiO2 1224.

In FIG. 12B (as also shown in FIG. 12E), it can be seen in the SpO2 plot 1226 that, between approximately minutes 101-106, there is rapid, significant oscillation in SpO2. The asymmetric nature of the derivative gain term 1234 (that is, tending to cause an increase in FiO2 when SpO2 is decreasing, but not tending to cause a symmetric decrease in FiO2 when SpO2 is increasing) here leads to a general increase of FiO2 during this time period, even though SpO2 is above target during this time period.

FIG. 12C shows the plots shown in FIG. 12A, over minutes 3-13. As can be seen, at around time 4-5 minutes, SpO2 1226 is below the target SpO2 1223, and decreasing. Therefore, the proportional gain term 1232 and the derivative gain term 1234 are large, and so the FiO2 correction 1236 is large, increasing the FiO2.

FIG. 12D shows the plots shown in FIG. 12A over minutes 53-63. At around time 59.8 minutes, SpO2 1226 is above the target SpO2 1223 and increasing, the proportional gain term 1232 is negative, and the derivative gain term 1234 is zero. As a result, the FiO2 correction 1236 is negative and equal to the proportional gain term (since the derivative gain term is zero), leading to a decreasing FiO2 1220.

FIG. 12E shows the plots shown in FIG. 12A, over minutes 95-115. It can be seen here (as also shown in FIG. 12B) that, in the SpO2 plot 1226, between approximately minutes 101-106, there is rapid, significant oscillation in SpO2. During this time period, the asymmetric nature of the derivative gain terms in the FiO2 corrections here lead to a series of FiO2 corrections, as shown in the FiO2 correction plot 1236, that cause an overall increase in FiO2 during this time period, as shown in FiO2 plot 1220. This is the case even though SpO2 is above the target SpO2 1293 during this time period. This result of the asymmetric gain term is advantageous, since the overall increase in FiO2 during this time period is the safest response to such an oscillation, which oscillation may be caused physiologically, or non-physiologically, such as by a noisy SpO2 signal that may be caused, for example, by motion, poor probe placement, light interference, or other reasons.

FIG. 13 is a flow diagram illustrating aspects of an example method 1300 operation of an example desaturation related algorithm, in accordance with embodiments of the present disclosure, which may, for example, be implemented by a ventilation system as disclosed herein.

At step 1304, the system confirms that the patient SpO2 signal is valid. If not, FiO2 closed loop control is disabled, such as until the validation can be obtained. If the SpO2 signal is validated, then the method 1300 proceeds to step 1306, where the system confirms that the ventilation system is confirmed as operational. Here also, if the ventilation system cannot be confirmed as operational, then, at step 1320, FiO2 closed loop control is disabled or paused, such as until the validation can be obtained. If the ventilation system is confirmed as operational, then the method 1300 proceeds to step 1308.

At step 1308, the method 1300 queries whether SpO2 (such as may be measured, for example, using an oximetry sensor 114, as depicted in FIG. 1, such as a pulse oximeter coupled with the patient), is below a lower threshold (e.g., 88%) for at least a threshold period of time, such as 0.25 seconds (or, e.g., between 0.25-1 second, between 1-5 seconds, between 5-15 seconds or between 15-30 seconds). If not, then, at step 1322 FiO2 closed loop control (e.g., aspects of one implementation of which are described with reference to FIG. 12A) is enabled (or continued) and, at step 1324, the method 1300 waits another increment of time. In some embodiments, the threshold period of time may function as a check to ensure that a short determined very low SpO2 is not incorrectly representative of the patient's SpO2, such as by being caused by movement, incorrect probe placement or artifact.

If, at step 1308, if is determined that SpO2 is below the lower threshold for at least the threshold period of time, then, at step 1310 FiO2 is set to 100% (however, in various embodiments, the new FiO2 setting may be, e.g., 80%-100%), and the algorithm proceeds from there. At step 1312, an alarm is displayed to inform the user that the desaturation threshold has been reached. In some embodiments, this may be done in order to urgently react to and minimize the duration and/or severity of the desaturation event.

Next, at step 1314, the method 1300 queries whether more than one desaturation event (however, in various embodiments, the number of desaturation events may be greater than 1, such as 2-5) has occurred in a threshold period of time (e.g., the last 20, 40 or 60 minutes). If not, then, at step 1324, the method 1300 waits another increment of time.

At step 1314, if it is determined that desaturation has occurred more than once in a threshold period of time (e.g., the last 20, 40 or 60 minutes), then, at step 1316, the target SpO2 is increased by a set amount, such as +0.01 (however, in various embodiments, the amount may be, e.g., between +0.01 and +0.03) and, at step 1318, an alarm is displayed to alert the user of this development (such as via a graphical user interface of a display of a portable ventilator 2000 as described with reference to FIGS. 20A-H). Next, at step 1324 the method 1300 waits another time increment.

FIGS. 14-17 provide a detailed illustration of determination of ventilation parameters based on calculated respiratory parameters, in accordance with embodiments of the present disclosure, which may, for example, be implemented by a ventilation system as disclosed herein.

FIG. 14 is a flow diagram 1400 illustrating aspects of an example method 1400 for determination of ventilation parameters based on calculated patient respiratory parameters and associated waveforms, in accordance with embodiments of the present disclosure.

In some embodiments, an equation relating to mechanics of the respiratory system is used to determine the patient's estimated Crs and may also be used to determine the patient's Rrs.

At step 1402, ventilation related patient respiratory dynamics data is obtained that is needed to determine required waveform data, for a selected number of patient breaths.

At step 1404, the obtained respiratory dynamics data is used to determine required waveforms and store the associated waveforms data.

At step 1406, the waveform data is used in determining values for respiratory mechanics parameters for the patient. In some embodiments, these may include Crs, or respiratory system compliance, and Rrs, or respiratory system resistance.

At step 1408, the determined values for Crs and Rrs are used in determining initial PIP, or PIP(0), when the ventilation system is in active mode.

Additional detail is provided as follows. An equation of motion for the respiratory system may be expressed as follows.

Paw(t)+Pmus(t)=(1/Crs)*V(t)+Rrs*Vdot(t)+PEEP+PEEPi (Equation 5)

In equation 5:
t=time
Paw=airway pressure
Pmus: pressure generated by the patient's inspiratory muscles
V: volume waveform
Vdot=flow waveform
PEEP=PEEP as applied by ventilator
PEEPi=Intrinsic PEEP (as known as auto-PEEP)
Crs: Respiratory system compliance
Rrs: Respiratory system resistance

For a passive patient, Pmus=0, and Equation 5 may simplify to the following.

Paw=(1/Crs)*V+Rrs*Vdot+Po (Equation 6)

In equation 6:
Po is a constant pressure term.
Using the above, for example, a least squares regression (or other mathematical calculation, model or fitting model) may be utilized and performed for each of several breaths in order to calculate estimated Crs, Rrs, and Po using Paw, V and Vdot waveforms.

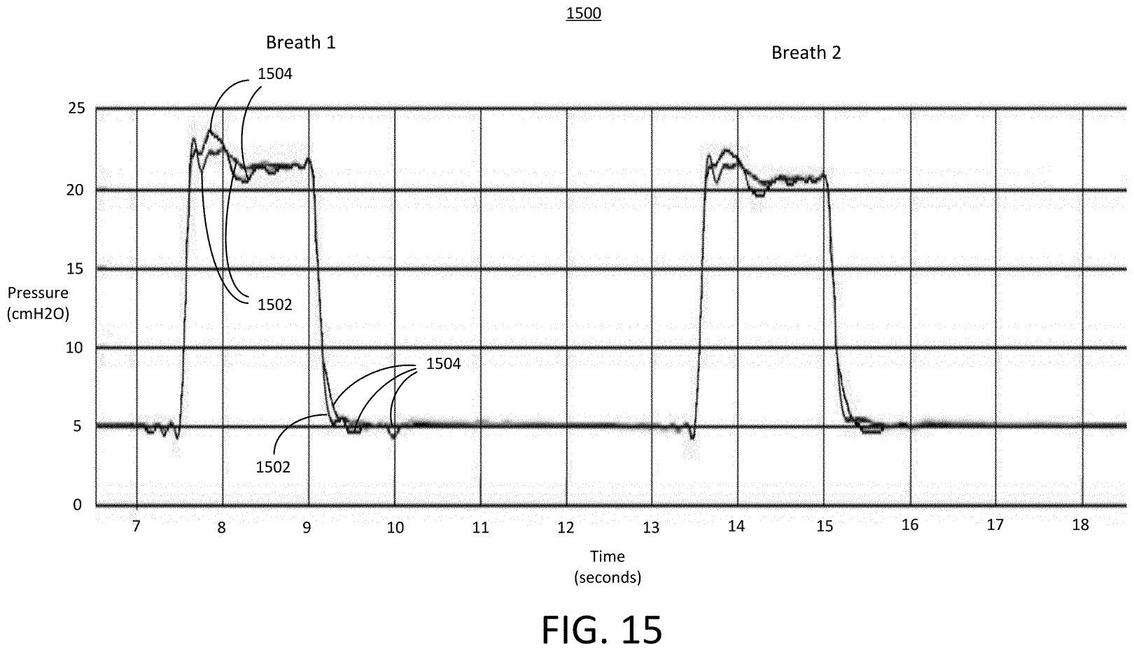

FIG. 15 illustrates waveforms 1500 including an example respiratory pressure waveform 1502 and related model fitted waveform 1504 that can be used in calculation of ventilation parameters, in accordance with embodiments of the present disclosure. In FIG. 15, waveform 1502 represents a respiratory pressure waveform generated from data obtained from two breaths delivered to a patient—breath 1 and breath 2. Waveform 1504 represents a model fitted waveform generated based on waveform 1502. For example, waveform 1504 may be generated based on a least squared regression algorithm performed on data from the waveform 1502.

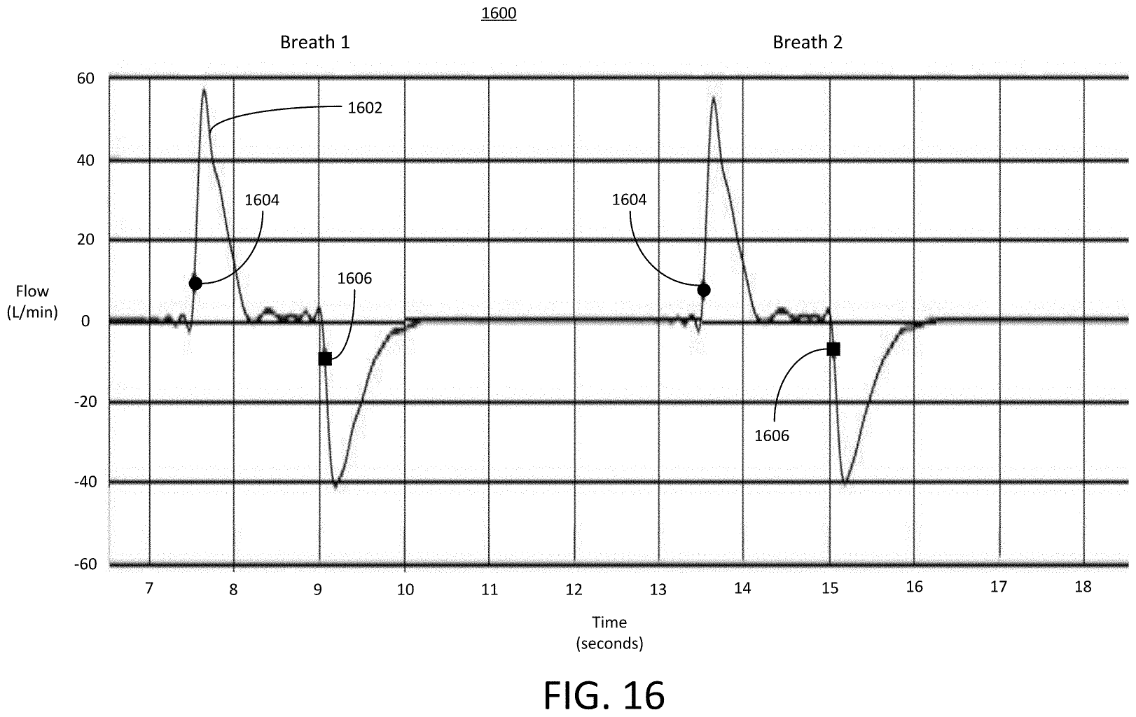

FIG. 16 provides an illustration 1600 of an example respiratory flow waveform 1602 that can be used in calculation of ventilation parameters, in accordance with embodiments of the present disclosure. In FIG. 16, waveform 1602 represents a respiratory flow waveform generated from data obtained from breath 1 and breath 2. In FIG. 16, markers 1604 represent the start of patient inspiration and markers 1606 represent the start of patient expiration.

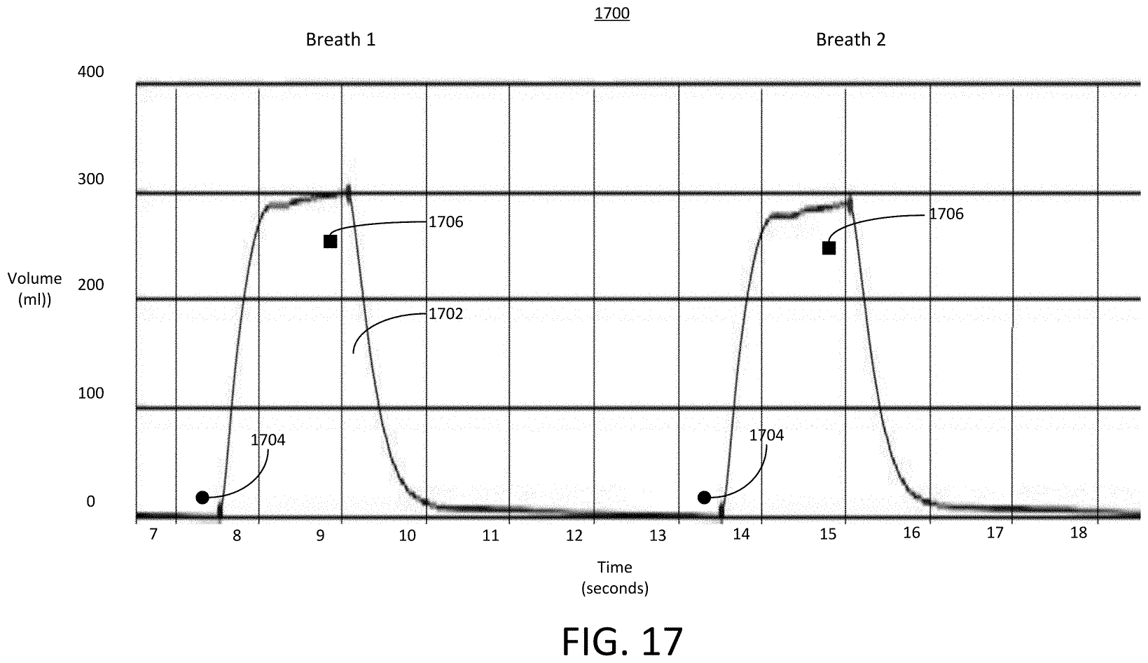

FIG. 17 provides an illustration 1700 of an example respiratory volume waveform that can be used in calculation of ventilation parameters. In FIG. 1700, waveform 1702 represents a respiratory flow waveform generated from data obtained from breath 1 and breath 2. In FIG. 17, markers 1704 represent the start of patient inspiration and markers 1706 represent the start of patient expiration.

In some embodiments, using data associated with the waveforms such as those depicted in FIGS. 14-16, and Equation 5, respiratory parameters may be calculated. For example, with regard to FIGS. 14-16, estimated respiratory parameters Crs and Rrs can calculated, with their values being as follows: Crs=18.4 ml/cm H2O, Rrs=13.1 cm H20/L/s, with Po=5 cm H2O. The calculated Crs and/or Rrs may be used in determining particular ventilation parameters, such as settings for PIP(0) and for Vt in active mode.

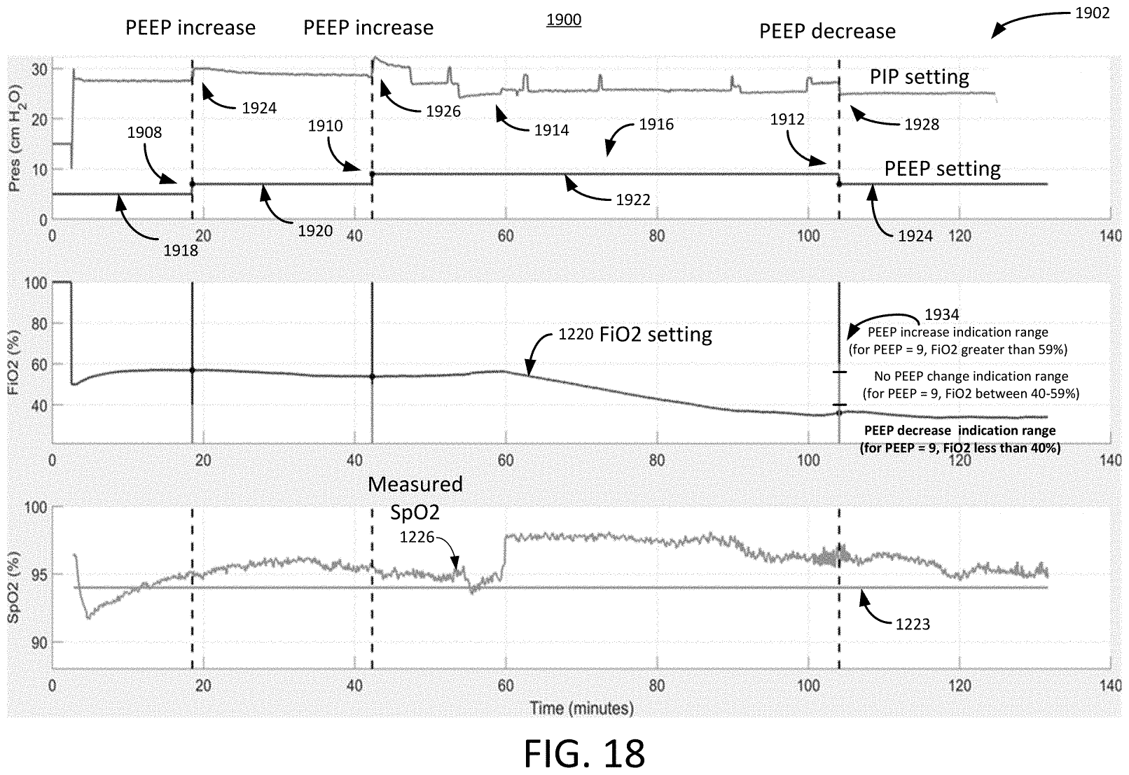

FIG. 18 illustrates example actual animal (porcine) study results 1900 demonstrating aspects of closed loop control, including FiO2 setting adjustment based on measured SpO2, and PEEP setting adjustment based on FiO2 setting, in accordance with embodiments of the present disclosure. FIG. 18 shows FiO2 1220, SpO2 1226 and the target SpO2 1223 of 94% (as also shown in FIG. 12B). The FiO2 setting is continually adjusted based on the SpO2. For example, from around time 4 minutes to around time 12 minutes, it can be seen that the SpO2 is fluctuating somewhat but remains substantially under the target SpO2 of 94%. During this time, FiO2 can be seen generally increasing in response to the below target SpO2. As a result, from around time 4 minutes to time 12 minutes, SpO2 fluctuates but generally increases until it eventually reaches and then exceeds the target SpO2 of 94%. Conversely, from around time 12 minutes to around time 33 minutes, in response to the over target SpO2, FiO2 can be seen generally decreasing.

Graph 1902 shows, in units of pressure (cm H2O), and over time (in minutes), the PEEP setting 1916 and the PIP setting 1914. PEEP increases 1908, 1910 occur at around times 18 minutes and 43 minutes, as well as a later PEEP decrease 1912 at around time 104 minutes. The PEEP setting can be seen varying from levels of 5 cm H2O 1918, to 7 cm H2O 1920, to 9 cm H2O 1922, and back to 7 cm H2O 1924. Sharp PIP increases can be seen 1924, 1926 corresponding to the PEEP increases 1908, 1910, and a sharp PIP decrease can be seen 1928 corresponding to the PEEP increase 1912.

Tracking the PEEP 1916 as shown in graph 1902 relative to FiO2 1930 in graph 1220 provides an illustration of closed loop control of the PEEP based on the FiO2, taking into account PEEP selection rules, one embodiment of which is illustrated with reference to FIG. 8.

As shown in graph 1902, PEEP starts out at 5 cm H2O. At around time 18 minutes, the PEEP selection rules (according to the embodiment depicted with reference to FIG. 8) and PEEP change eligibility rules lead to a PEEP increase from 5 to 7 cm H2O. In particular, at around time 18 minutes, FiO2 is over 39%, so the PEEP selection rules indicate a PEEP increase by one level, from 5 to 7 cm H2O, since the current FiO2 is outside of and above the FiO2 range of 21-39% that corresponds to the current PEEP of 5 cm H2O. This may generally indicate that the subject's lungs became increasingly “sick” or functionally impaired, which led to a higher FiO2, which then led to an increase in PEEP.

Similarly, at around time 42 minutes, FiO2 is over 49%, leading to a PEEP increase by one more level, from 7 to 9 cm H2O, since the current FiO2 is outside of and above the range of 30-49% that corresponds to a PEEP of 7 cm H2O, and since PEEP change eligibility conditions here are met that require that, for a PEEP increase, the PEEP has not changed for at least a predetermined period of time (in this case, 10 minutes) as well as a steady state FiO2 for at least a predetermined period of time (in this case, 10 minutes).

Later, at around time 103 minutes, FiO2 is under 40%, leading to a PEEP decrease by one level, from 9 back to 7 cm H2O, since the current FiO2 is outside of and below the range of 40-59% that corresponds to a PEEP of 9 cm H2O, and since PEEP change eligibility conditions here are met that require that, for a PEEP decrease, the PEEP has not changed for at least a predetermined period of time (in this case, one hour) as well as a steady state FiO2 for at least a predetermined period of time (in this case, 10 minutes). For illustration purposes, relevant FiO2 ranges associated with PEEP of 9 are noted 1934 on the graph 1220. This generally indicates that the animal's lungs are getting less “sick” or functionally impaired, which led to a lower FiO2, which then led to a decrease in PEEP.

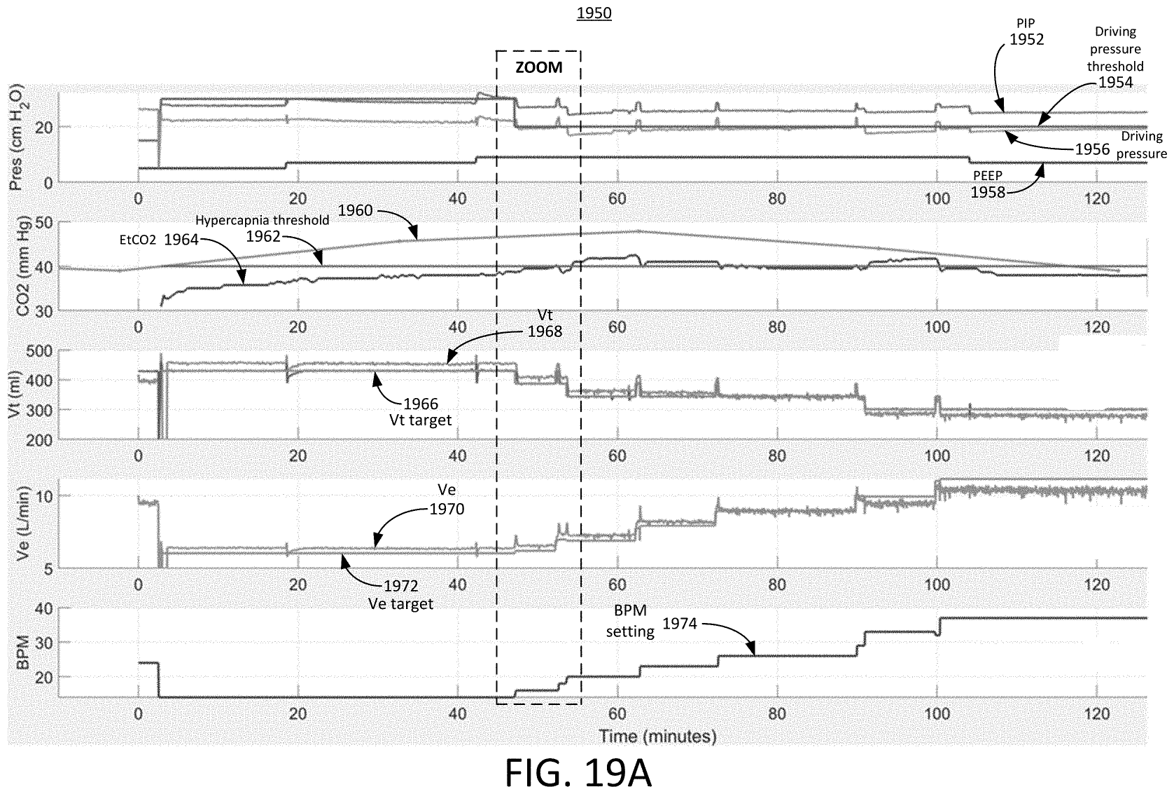

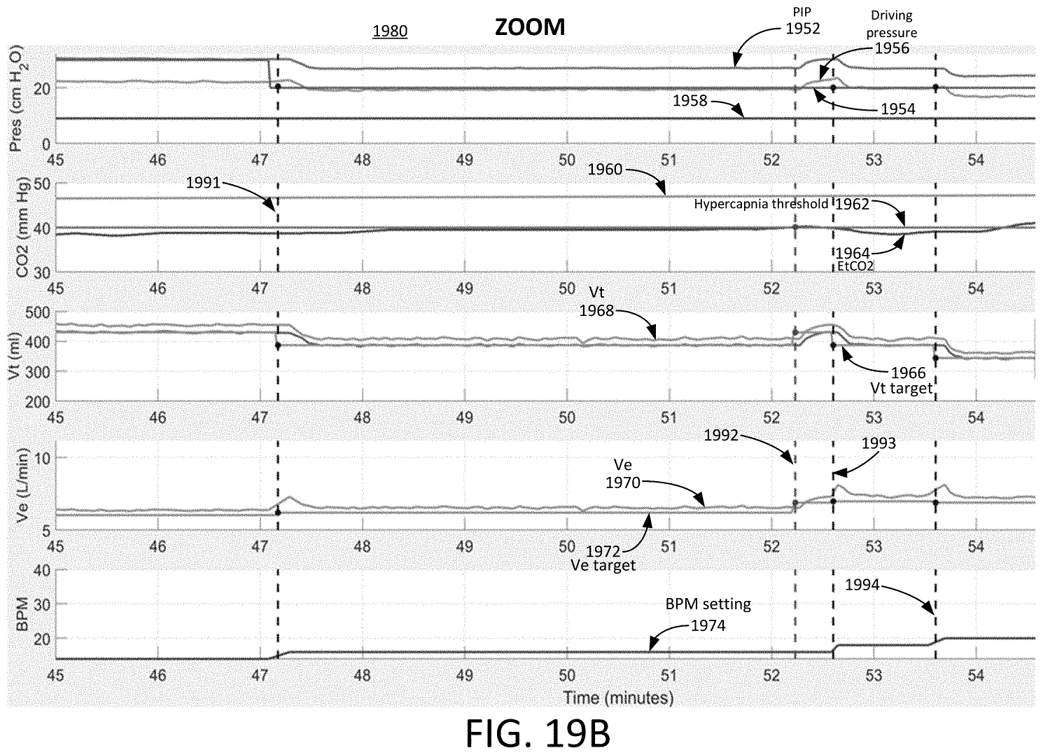

FIGS. 19A-19B illustrates example animal (porcine) study results 1950, 1980 demonstrating aspects of closed loop control, including FiO2 settings adjustment based on EtCO2, in accordance with embodiments of the present disclosure.

FIG. 19A shows (over time 1-120 minutes), and FIG. 19B shows (zoomed over time 45-54 minutes) PIP 1952, measured driving pressure 1956, driving pressure threshold 1954, PEEP 1958, blood gas CO2 partial pressure 1960, measured EtCO2 1964, hypercapnia threshold 1962, measured tidal volume 1968, tidal volume target 1966, measured minute ventilation 1970, minute ventilation target 1972, and breath rate setting 1974, in breaths per minute (BPM). FIGS. 19A and 19B show results corresponding to embodiments described, for example, with reference to FIG. 4 herein. In particular, as shown in FIG. 19B, in response to EtCO2 rising above what is here the hypercapnia threshold 1984 (here, 40 mm Hg), Ve can be seen appropriately increasing, whereas, when EtCO2 is in what is here the normocapnia range (between 25-40 mm Hg), PIP is adjusted based on Vt relative to target Vt.

In FIG. 19A, from approximately minutes 0-50, it can be seen that EtCO2 1964 is, overall, increasing. This can indicate that the subject's lungs are not efficiently removing CO2 from the body, i.e., the respiratory system is not able to keep up with the need to remove CO2.

PEEP 1958 is seen increasing twice during this period—once at approximately time 18 minutes, from the initial PEEP setting of 5 cm H2O to the next higher PEEP level of 7 cm H2O, and then again at approximately time 42 minutes, from 7 cm H2O to the next higher level of 9 cm H2O. It is also notable that, much later, at approximately time 104 minutes, PEEP can be seen decreasing from 9 cm H2O to 7 cm H2O. These PEEP increases and decreases are in accordance with a particular embodiment of PEEP selection rules (where a PEEP change is indicated based on FiO2 and a current PEEP level, as described with reference to FIG. 8) as well as a particular embodiment of PEEP eligibility rules (as described with reference to FIGS. 6 and 7—here, PEEP is eligible for increase only if PEEP has not changed in at least the last 20 minutes, and PEEP is eligible for decrease only if PEEP has not changed in the last one hour).

SpO2 and FiO2 are not shown in FIG. 19A, but are shown and described with reference to FIG. 18. As shown in FIG. 18, FiO2 is generally high enough over approximately minutes 1-42 to lead to the two increases in PEEP, in accordance with the PEEP selection rules. By approximately minute 104, FiO2 is much lower (generally indicating that the subject is in need of less support), which leads to the PEEP decrease at approximately minute 104.

In FIG. 19B, during approximately minutes 45-54, a number of observations can be made. Early in this period, PIP setting (1952) and driving pressure (1956) are relatively high. Between minutes 47 and 48, at approximately the time indicated by dotted line 1991, it can be seen that Vt target (1966) decreases in response to a driving pressure above the driving pressure limit (1954). The PIP setting to achieve this decreased target Vt and therefore the measured Vt also decreases. Since Vt is decreasing, to keep Ve steady, RR must increase—accordingly, it can be seen that the BPM setting (1974) increases during this period. However, during this period, EtCO2 is increasing, indicating that the subject requires more ventilation.

At just after minute 52, as indicated by dotted line 1992, EtCO2 passes above the hypercapnia threshold. As a result, it can be seen that target Ve (1972) increases by increasing the target Vt. To achieve the increased target Vt, the PIP setting and therefore driving pressure increase. This increased driving pressure almost immediately results in the driving pressure exceeding the threshold, as indicated by line 1993. As a result, the target Vt decreases back to its previous level and the BPM setting is increased to maintain the same target Ve. This additional support causes EtCO2 to decrease during this time period. As indicated by dotted line 1994, between minutes 53 and 54, driving pressure again exceeds the threshold and, as a result target Vt decreases. As before, to maintain a constant target Ve, RR increases as shown by an increase in BPM setting.

FIGS. 20A-H illustrate simplified example portable ventilators, displays, and display aspects, in accordance with embodiments of the present disclosure.

As shown in FIG. 20A, the portable ventilator 2000 includes features such as pulse oximeter connector 2001, fresh gas/emergency air intake 2002, handle 2006, power switch 2009, battery compartment 2010, user selection dial 2011, control panel 2012, manual breath/plateau pressure button 2013, menu button 2019, oxygen inlet 2017 and display and user interface screen 2016.

The fresh gas/emergency air intake 2002 provides a gas path and allows ambient air into the device's internal compressor. Built-in filters are used to protect the compressor and patient from particulate matter. The intake 2002 also acts as an anti-asphyxia valve that enables the patient to breathe ambient air, should the ventilator fail. The intake 2002 further contains a particulate filter and permits the user to connect either a bacteria/viral or a chemical/biological filter, depending on ambient conditions. Furthermore, an oxygen reservoir bag assembly may be connected to the intake 2002 to allow for low flow oxygen use with the ventilator 2000 in order to provide a source of supplemental oxygen to patients during ventilation. For example, low flow oxygen sources can be obtained based on a flow meter or an oxygen concentrator. Oxygen may be delivered through the intake 2002 when the ventilator's internal compressor cycles deliver breaths.

A top panel of the ventilator 2000 may have components including, in addition to the intake 2002 and the pulse oximeter connector 2001, a high pressure oxygen input, a gas output, a power cord connector for external AC/DC power, a USB port, an exhalation valve, an exhaust valve and a transducer. The pulse oximeter connector 2001 is used to connect a pulse oximeter that may provide continuous non-invasive monitoring of SpO2 and pulse rate. The portable ventilator 2000 may be operable using external AC/DC power or a battery, such as an internal lithium ion battery.

Furthermore, the portable ventilator 2000 may include at least one display and user interface screen 2016, which may, for example, include a liquid crystal display (LCD). Among other things, the display and user interface 2016 may provide a user with data relating to patient parameters and ventilation parameters, including current ventilator settings, which may be continuously updated. Furthermore, the display and user interface 2016 may include, among other things, various graphical user interface (GUI) aspects, allowing user interaction, such as to access particular data, change ventilator selections or settings, confirm suggested displayed changes to ventilator settings, receive and respond to alarms, etc. In particular, as shown, the display and user interface screen 2016 includes parameter and alarm indicators 2007, an alarm message center/waveform window 2018, parameter buttons 2008 and auxiliary parameter boxes 2014.

In some embodiments, the display and user interface screen 2016 may be divided into a number of sections. For example, as depicted, the top left area of the display and user interface screen 2016 may include airway pressure, flow, volume, capnography and plethysmography (pleth) waveform plots. This section may include displayed plots for airway pressure as well as, when a pulse oximeter is connected, the pleth waveform, and when a CO2 sensor is connected, the capnogram. When a plot is useful to facilitate a parameter adjustment by the user, a message area may display both the plot and a context menu that the user may use to make selections to obtain displayed context relating to the parameter.

The display and user interface screen 2016 also includes a menu display section in the top left area. This section may be used to display a menu after the user presses a menu button on the ventilator's control panel, and may be used to display context menus associated with particular parameters.

The display and user interface screen 2016 also includes an alarm message center/waveform window 2018 in the upper left area, in which visible alarms may at times be displayed. Some alarms may instruct the user to consult a physician, for example. In some embodiments, alarms may be categorized into different levels of priority, such as based on the level and/or urgency of the risk that the particular alarm condition may pose to the patient. Multiple alarms, along with their priorities, that have occurred recently may be available for display to a user, where the user may view the recent alarms by scrolling in a GUI, for example. In some embodiments, if the FiO2 setting is increased by a certain amount, such as 10% (or, e.g., 5-15%) during a predetermined period of time, such as 10 minutes (or, e.g., 5-15 minutes), an alarm is generated. Furthermore, in some embodiments, certain alarms may cause some or all closed loop control aspects, or VPFC mode including FiO2 and PEEP closed loop control, to pause until the user clears the alarm. In some embodiments, during the time of the pause and before the alarm is cleared, the ventilator may operate using current parameter values, such as for FiO2 and PEEP, that were being used at the time that the pause was initiated.

The display and user interface screen 2016 may also include pop-up windows that may provide a user with context-sensitive guidance, such as in connection with manual adjustment of parameter values, for example.

The display and user interface screen 2016 also includes various parameter windows on the right side. Displayed parameters may include, e.g., SpO2, EtCO2, FiO2, PEEP, PIP, Vt, BPM and blood pressure, for example. Each parameter window may display a primary parameter as well as secondary parameters, such as parameters that may be related to the primary parameter or with associated alarm limits. In some embodiments, solid text may be displayed for primary and secondary parameter values that can be adjusted by the user, while outlined text may be used for patient-dependent parameters, for example. Primary parameters may also include mode, which may include a user selectable mode of operation including assist/control (AC), SIMV (synchronized Intermittent Mandatory Ventilation), continuous positive airway pressure (CPAP) and bilevel.

Furthermore, the mode parameter may be associated with secondary parameter choices including volume targeting and pressure targeting. Volume targeting (V) assures that a constant volume is delivered to the patient in the inspiratory time using a constant flow. During volume targeting, the measured PIP parameter is displayed or highlighted. Pressure targeting (P) assures a constant airway pressure for the duration of the inspiratory time. During pressure targeting, the measured Vt parameter is displayed or highlighted.

The display and user interface screen 2016 also includes device-related icons section in the lower left area. This section may include icons that represent, and may provide status information on, for example, the ventilator's power source (which may indicate whether the ventilator is operating on external power or its battery), a battery charging status icon, an oxygen supply attachment icon, and an icon that indicates whether audible alarms or permitted or muted.

The display and user interface 2016 may also include an auxiliary parameter boxes section 2014, which may be located toward the bottom. This section may display parameter boxes that allow the user to adjust a particular parameter using a context menu associated with the parameter.

In some embodiments, a user may take the following steps in setting up. The patient circuit may be attached to the ventilator's top panel. A high pressure oxygen supply, if it is to be used, is attached. The user inspects the fresh gas/emergency air intake filters and may attach other items, such as an oxygen reservoir bag, and biological and chemical filters. The user may choose a power source, such as an external or internal power source. The user may connect the power supply to the ventilator. Once preliminary steps are completed, the user may power on the ventilator 2000 using the ventilator's power switch or button. Once powered on, the ventilator 2000 may perform a self-check, to check for potential alarm conditions as well as the operation of the pneumatic system, power system and internal communications system. During normal start-up, the ventilator's alarms may be muted for, e.g., 2 minutes, to allow the user to connect items including the patient circuit and pulse oximeter, and to perform operational tests.

In some embodiments, after powering on the ventilator 2000, the user may choose from the settings defaults, such as adult, pediatric, mask CPAP, custom (includes use of saved settings values), and last settings (includes use of last-used settings values). The user may select one of the defaults, in which case ventilation will be initiated using the default settings associated with the selection. Alternatively, the user may manually set settings using parameter buttons of the display 2030. Furthermore, the user may select a mode of operation, including, as described briefly above, AC, SIMV, CPAP or bilevel. In AC, the patient receives either controlled or assisted breaths. When the patient triggers an assisted breath, the patient receives a breath, and either a pressure target or a volume target is utilized. In SIMV, the patient receives controlled breaths based on the setting of the breathing rate. In CPAP, the patient receives a constant positive airway pressure while breathing spontaneously. Spontaneous breaths may be either unsupported demand flow or supported using pressure support. In bilevel mode, the ventilator 2000 provides two pressure settings to assist the patient in breathing spontaneously, including a higher inspired positive airway pressure (IPAP) and a lower expiratory positive airway pressure (EPAP).

The parameter and alarm indicators 2007 may include information specifying current parameter values, and may also display other related information such as alarm threshold values 2028, which may indicate, for example, threshold beyond which an alarm may be triggered.

The parameter and alarm indicators 2007 may include an SpO2 parameter display aspect 2003, an FiO2 parameter display aspect 2004 and a PEEP parameter display aspect 2005.

In the SpO2 parameter display aspect 2003, the current patient SpO2 is displayed as “95”, meaning 95%. A target symbol 2035, along with the displayed numbers “94” and “88” indicate that the SpO2 target is set to 94% and the desaturation threshold of SpO2 is set to 88%.

In the FiO2 parameter display aspect 2004, the current FiO2 is displayed as “99”, meaning 99%. Double arrows 2015 (which, in some embodiments, may be animated as displayed), indicate that FiO2 closed loop control is currently turned on and operating. The displayed “O2 in use” text indicates that an attached oxygen supply is being used.

In some embodiments, various alerts or warnings may be displayed to the user on the display and user interface screen 2016 before the user initiates FiO2 closed loop control. For example, the user may be warned not to use FiO2 closed loop control if the user suspects that pulse oximetry may not operate correctly or may not be available, or if the patient has carboxyhemoglobin poising (i.e., carbon monoxide poisoning), in which case the user may be advised to follow the local standard of care. The user may also be warned not to use FiO2 closed loop control for patients with a core temperature of less than 35 degrees Celsius. Furthermore, in some embodiments, pulse oximetry and a high pressure oxygen source may be required for FiO2 closed loop control, and failure of availability of either of these resources may cause FiO2 closed loop control to be paused and may cause an associated alarm to be displayed to the user.

In the PEEP parameter display aspect 2005, the current PIP is displayed as 28, meaning 28 cm H2O, and the current PEEP is displayed at 5, meaning 5 cm H2O. PIP alarm threshold levels are also displayed.

FIG. 20B illustrates a simplified example portable ventilator and display, with FiO2 closed loop control paused, whether by user selection or by the ventilator 2000 without user selection. A pause symbol 2025 replaces the double arrows 2015, as displayed in FIG. 20A, to indicate that FiO2 closed loop control has been paused. Also, “FiO2 CLC paused” is displayed. Furthermore, additional associated information 2026 may also be displayed. As described with reference to FIG. 13, FiO2 may be paused if the ventilator cannot confirm that it is operational or if the patient SpO2 signal cannot be confirmed as valid, or may also be paused at user selection. In some embodiments, the displayed additional information 2012 may include a particular detected alarm condition, or associated code, which led to the pause in FiO2 closed loop control. The displayed additional information may also include may one or more messages that may instruct the user to make certain checks, such as a check that the oxygen source pressure is correct or that the pulse oximeter is properly connected, for example. In some embodiments, FiO2 closed loop control does not resume until any associated alarm conditions are resolved and cleared.

In some embodiments, alarm conditions that can lead to pause in FiO2 closed loop control can be of various types. For example, some alarms may relate to a failure of a pneumatic system ventilator self-check, such as may relate to the compressor or oxygen flow paths, an internal communication failure, or a failure relating to a sensor, transducer or associated signal. Some alarms may relate to the conditions described with reference to FIG. 13, such as if the ventilator cannot confirm that it is operational or if the patient SpO2 signal cannot be confirmed as valid.

FIG. 20C illustrates a simplified example portable ventilator and display, with PEEP closed loop control enabled. Double arrows 2020 (which, in some embodiments, may be animated as displayed), indicate that PEEP closed loop control is currently turned on and operating.

FIG. 20D illustrates a simplified example portable ventilator and display, with PEEP closed loop control paused, whether by user selection or by the ventilator 2000 without user selection. A pause symbol 2030 replaces the double arrows 2020, as displayed in FIG. 20C, to indicate that PEEP closed loop control has been paused. Also, “PEEP CLC paused” is displayed. Furthermore, additional associated information 2032 may also be displayed.

FIG. 20E illustrates a simplified example portable ventilator and display, with FiO2 closed loop control and PEEP closed loop control both turned on and operating, as indicated by double arrows 2040 and 2042.

FIG. 20F illustrates a simplified example portable ventilator and display, with FiO2 and PEEP closed loop control both paused, as indicated by the displayed pause symbols 2050 and 2052. Also, “FiO2 and PEEP CLC paused” is displayed. Furthermore, additional associated information 2054 may also be displayed.

Although not depicted, FiO2 closed loop control may be paused (and so indicated) while PEEP is turned on and operating (and so indicated), or PEEP closed loop control may be paused (and so indicated) while FiO2 closed loop control is turned on and operating (and so indicated).

FIG. 20G illustrates a simplified example displayed menu 2060 of a portable ventilator display. In the embodiment depicted, the menu 2060 includes displayed aspects 2064 for FiO2 closed loop control (indicated as “on”, meaning turned on and operating), PEEP closed loop control (indicated as “on”, meaning turned on and operating), 02 reservoir (indicated as “on”, meaning connected), SpO2 target (indicated as “94”, meaning 94%) and desat (desaturation) threshold (indicated as “88”, meaning 88%). In various embodiments, some or each of the displayed aspects 2064 are selectable by the user, such as to change “on” to “off” to pause, or turn back on, FiO2 closed loop control or PEEP closed loop control, or to change the SpO2 target or desat threshold. In various embodiments, the various displayed aspects 2064 of the menu 2060, or various combinations thereof, may be separately included in multiple different menus. In various embodiments, menus may be accessed by a user in various different ways, such as by pressing and holding an appropriate one or more of the parameter buttons 2008, and user selections may be made in various ways, such as may include use of the user selection dial 2011 and/or pressing of one or more physical or GUI buttons, for example.

FIGS. 20H-I illustrate simplified example displayed messages 2070, 2080 of portable ventilator displays. In some embodiments, once the user turns on FiO2 closed loop control or PEEP closed loop control, a popup message may be displayed that indicates that FiO2 closed loop control has been turned on 2072 or that PEEP closed loop control has been turned on 2082, or both. The display may also provide one or more additional messages or reminders 2074, 2084, such as to remind the user than FiO2 closed loop control or PEEP closed loop control will continue to adjust automatically, but that the user can elect to manually turn off FiO2 closed loop control or PEEP closed loop control, and control FiO2 and/or PEEP manually 2082, 2084.

FIG. 21 illustrates aspects of an example pneumatic system 2100 that can be used with a portable ventilator, in accordance with embodiments of the present disclosure. As shown, the pneumatic system 2100 includes an oxygen connector 2110, patient circuit tubing 2112 that may include patent inhalation and exhalation circuit components, an exhalation valve 2114, a radial compressor 2108 or other gas moving component such as a blower, an oxygen valve 2106, and components 2104 associated with control and/or measurement of oxygen pressure, oxygen flow, ambient pressure, intake pressure and patient airway pressure, which may include programmable-gain amplifiers (PGAs), analog-to-digital converters (ADCs), and other components.

The oxygen valve 2106 and the compressor 2108 may provide the appropriate gas mixture for the patient during ventilation. The system 2100 may include transducers for pressure measurements including oxygen input supply and barometric pressure. The patient circuit tubing 2112 may include an inspiratory portion that provides gas to the patient, as well as an expiratory portion that exhausts gas directly to the atmosphere without return to the ventilator 2000. The ventilator 2000 pneumatically controls the exhalation valve 2114. A transducer within the ventilator 2000 measures the airway pressure during ventilation.

An external high pressure gas source connects to the ventilator 2000 using a high pressure oxygen input port. The source may be a medical grade oxygen system or oxygen cylinder supply, for example.

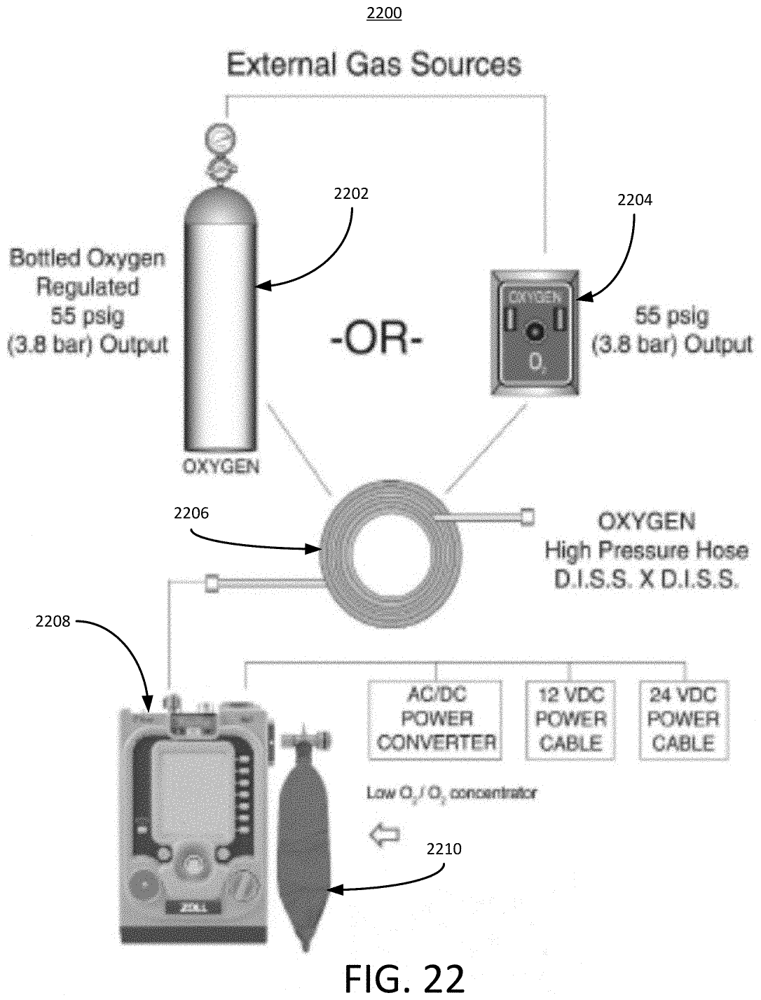

In some embodiments, a portable ventilator, such as, for example, the portable ventilator 102, as depicted in FIG. 1, or the portable ventilator 2000, as depicted in FIG. 20A, may be coupled with a supplemental oxygen source (some examples of types of oxygen sources are provided with reference to FIG. 22). In various embodiments, the portable ventilator may be capable of supplying oxygen, using the supplemental oxygen source, in a number of different ways, or the portable ventilator may be capable of supplying oxygen in any one of several different ways. In various embodiments, the manner in which oxygen is supplied may be determined without user selection, or may be selected by a user, and may or may not require user confirmation.

In some embodiments, a reservoir bag may be used that allows entrainment of oxygen from a low pressure oxygen source. For example, a user may adjust the flow rate of oxygen based at least in part on current SpO2 (such as, for example, may be measured using an oximetry sensor 114, as depicted in FIG. 1, such as a pulse oximeter coupled with the patient).

In some embodiments, the portable ventilator includes and uses a variable rate regulatory valve, in which an oxygen output rate allowed or facilitated by the variable rate regulatory valve may be varied and changed to a particular oxygen flow rate of a range of possible oxygen flow rates, such as may range from 0 l/min to 200 l/min (or, e.g., up to 220, 250 or 275 l/min). The variable rate regulatory valve may be used in providing a variable and controllable oxygen flow rate for gas provided by a gas delivery apparatus (such as, for example, gas delivery apparatus 106, as depicted in FIG. 1) of the portable ventilator, such as using a facemask 110, as depicted in FIG. 1, coupled with the patient, or intubation, during the providing of mechanical ventilation. In some embodiments, the variable rate regulatory valve may attach to a gas inlet (such as, for example, oxygen inlet 2017, as depicted in FIG. 20A) of the portable ventilator and a high pressure oxygen source, and potentially one or more other devices and/or sensing or monitoring components.

In some embodiments, the variable rate regulatory valve may be controlled automatically or without need for user interaction. For example, this may be based at least in part on the patient's continuously monitored SpO2 and in accordance with an FiO2 setting or adjustment that may be determined based at least in part on the current SpO2. In some embodiments, this arrangement may be used in providing FiO2 CLC. In some embodiments, a portable oxygen concentrator (POC) may be used. FiO2 CLC may be used in controlling and regulating the output of the POC for entrainment of oxygen into the gas delivery apparatus during mechanical ventilation. Furthermore, in some embodiments, including embodiments in which the variable rate regulatory valve or a POC is used, PEEP CLC may also be included in control of the gas delivery apparatus. In some embodiments, PEEP CLC may be based least in part on a current FiO2 setting and a current PEEP setting.

FIG. 22 illustrates aspects of an example external gas supply system 2200 that can be used with a portable ventilator 2208, in accordance with embodiments of the present disclosure. Depicted components include an oxygen tank 2202 or other oxygen output 2204, high pressure hose for use with oxygen supply 2206, and an oxygen tank 2210 shown coupled to the portable ventilator 2208.

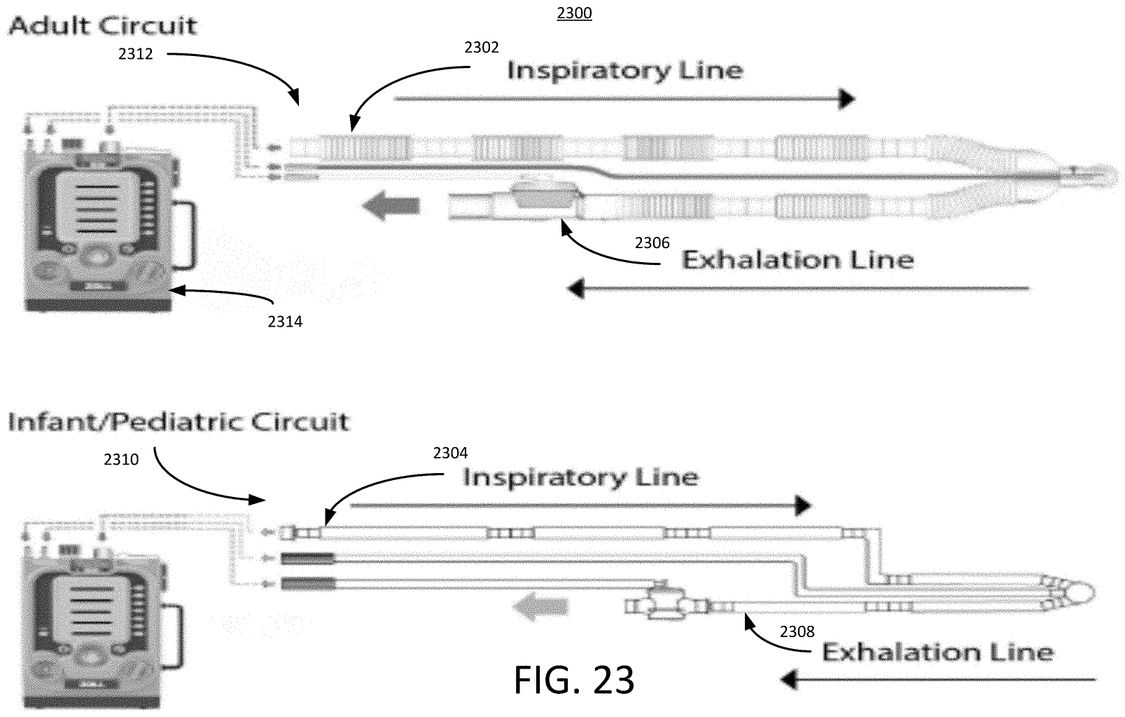

FIG. 23 illustrates aspects of example patient circuits 2300 that can be used with a portable ventilator 2314, in accordance with embodiments of the present disclosure. Depicted components include an adult circuit 2312, including an inspiratory line 2302 and an expiratory line 2306, and an infant/pediatric circuit 2310, including an inspiratory line 2304 and an expiratory line 2308.

1. A mechanical ventilator apparatus, comprising:
a gas delivery apparatus, having a patient interface, configured to deliver gas to a patient;
an oximetry sensor configured to generate signals representative of an oxygen concentration of the patient's blood; and
a controller, comprising a processor and a memory, in communication with the gas delivery apparatus and the oximetry sensor, the controller being configured to:
control the gas delivery apparatus to deliver the gas to the patient according to a FiO2 setting and a PEEP setting, wherein the FiO2 setting and the PEEP setting are configured to be adjustable,
control the delivery of the gas to the patient according to a first FiO2 value and a first PEEP value,
receive the signals representative of the oxygen concentration of the patient's blood from the oximetry sensor during the delivery of the gas to the patient,
determine the oxygen concentration of the patient's blood based at least in part on the received signals,
based at least in part on the oxygen concentration of the patient's blood, control the gas delivery apparatus to adjust the FiO2 setting to an updated FiO2 setting, and
based at least in part on the updated FiO2 setting, control the gas delivery apparatus to adjust the PEEP setting to an updated PEEP value.

2. The mechanical ventilator apparatus of claim 1, comprising controlling the delivery of the gas to the patient according to a first FiO2 value for the FiO2 setting and comprising controlling the delivery of the gas to the patient according to a first PEEP value for the PEEP setting.

3. (canceled)

4. The mechanical ventilator apparatus of claim 1, wherein the oximetry sensor comprises a pulse oximetry sensor comprising an SpO2 sensor, wherein the oxygen concentration of the patient's blood is an oxygen saturation, wherein the gas is a breathing gas.

5-7. (canceled)

8. The mechanical ventilator apparatus of claim 1, wherein the updated PEEP value is determined based at least in part on the updated FiO2 setting and the first PEEP value.

9. The mechanical ventilator apparatus of claim 1:
wherein the adjustment to the FiO2 setting comprises an adjustment to the FiO2 setting from a first FiO2 level to a second FiO2 level,
wherein the adjustment to the PEEP setting comprises an adjustment in the PEEP setting from a first PEEP level to a second PEEP level, and
wherein the determined PEEP update is based at least in part on:
the second FiO2 level, and
the first PEEP level.

10. The mechanical ventilator apparatus of claim 1, wherein the PEEP setting is adjusted based on a selection from at least two PEEP levels comprising a first PEEP level associated with a first FiO2 range and a second PEEP level associated with a second FiO2 range, wherein the first FiO2 range overlaps with the second FiO2 range,
wherein the PEEP update is determined so as to differ from the PEEP setting if one or more conditions are met,
wherein the one or more conditions comprise that a level of FiO2 of the gas being delivered to the patient has changed so as to fall outside of the first FiO2 range.

11. The mechanical ventilator apparatus of claim 10, wherein the adjustment to the PEEP setting comprises a change in the PEEP setting from the first PEEP level to the second PEEP level.

12. The mechanical ventilator apparatus of claim 1, wherein a change in the PEEP setting is based on a set of one or more PEEP change eligibility conditions being met, the set of conditions comprising that:
the FiO2 setting has not changed by at least a first amount in at least a first specified period of time or the level of SpO2 of the patient has been below a desaturation threshold for more than a second specified period of time.

13. The mechanical ventilator apparatus of claim 12, wherein the set of conditions comprises:
if the determined PEEP update comprises an increase in PEEP, the PEEP setting has not changed over a third period of time, and
if the determined PEEP update comprises a decrease in PEEP, the PEEP setting has not changed over a fourth period of time, the third period of time being different than the fourth period of time.

14. The mechanical ventilator apparatus of claim 13, wherein the fourth period of time is greater than the third period of time.

15. The mechanical ventilator apparatus of claim 1, wherein a change in the PEEP setting is based at least in part on a set of one or more PEEP change eligibility conditions being met, the set of conditions comprising that:
if the determined PEEP update comprises an increase in PEEP, the PEEP setting has not changed over a first period of time, and
if the determined PEEP update comprises a decrease in PEEP, the PEEP setting has not changed over a second period of time, the second period of time being different than the first period of time.

16. (canceled)

17. The mechanical ventilator apparatus of claim 1, wherein a change the PEEP setting is based on a set of one or more PEEP change eligibility conditions being met, the set of conditions comprising that:
if the determined PEEP update comprises an increase in PEEP, one or more measures of a hemodynamic status of the patient indicate that the hemodynamic status of the patient is above a first threshold.

18. The mechanical ventilator apparatus of claim 1, wherein adjusting the FiO2 value comprises:
determining a decrease in the oxygen concentration of the patient's blood from a previous time or time period to a current time or time period;
determining a correction value, wherein the correction value is increased based at least in part on the determined decrease in the oxygen concentration of the patient's blood; and
adjusting the FiO2 value by adding the correction value to the FiO2 setting.

19. The mechanical ventilator apparatus of claim 18, wherein adjusting the FiO2 setting comprises creating a tendency for the FiO2 to change so as to cause the SpO2 to approach a target SpO2.

20. The mechanical ventilator apparatus of claim 1, wherein the controller is configured to estimate a respiratory system compliance (Crs) of the patient and update a peak inspiratory pressure (PIP) setting of the ventilator apparatus based at least in part on the estimated Crs of the patient, and wherein the controller is configured to estimate the Crs of the patient based at least in part on application of at least one data fitting algorithm to a set of waveforms associated with respiratory mechanics of one or more breaths administered to the patient.

21. (canceled)

22. The mechanical ventilator apparatus of claim 1, wherein the controller is configured to, based at least in part on a determination that a driving pressure or plateau pressure being delivered to the patient is over a P threshold, decrease a tidal volume (Vt) delivered to the patient, wherein the controller is configured to, based at least in part on the decreased Vt delivered to the patient, increase a respiration rate (RR) delivered to the patient, wherein the controller is configured to increase Ve by increasing at least one of Vt and RR.

23-24. (canceled)

25. The mechanical ventilator apparatus of claim 1, wherein the controller is configured to, based at least in part on a Vt being delivered to the patient that is below a Vt threshold, trigger a low Vt alarm.

26. The mechanical ventilator apparatus of claim 1, wherein a Ve setting is configured to be adjustable based at least in part on a determined carbon dioxide concentration or partial pressure of the expired gas of the patient.

27. The mechanical ventilator apparatus of claim 1, wherein the controller is configured to, based at least in part on a capnographic measure that is above a threshold, increase a Ve being delivered to the patient, wherein the capnographic measure is at least one of: an EtCO2 measure and a measure obtained using a capnography sensor.

28-29. (canceled)

30. The mechanical ventilator apparatus of claim 20, wherein the controller is configured to, based at least in part on a predicted or ideal bodyweight of the patient determined based at least in part a gender and a height of the patient, determine a set of initial ventilation parameters for the one or more breaths administered to the patient, the initial ventilation parameters including an initial Vt setting or an initial PIP setting used for the one or more breaths administered to the patient.

31. The mechanical ventilator apparatus of claim 1, wherein a minimum PEEP setting of the gas delivery apparatus is between 0 cm water (H2O) and 10 cm H2O, wherein a maximum PEEP setting of the gas delivery apparatus is between 10 cm H2O and 20 cm H2O.

32. The mechanical ventilator apparatus of claim 1, wherein a minimum PEEP setting of the gas delivery apparatus is 5 cm H2O, wherein a maximum PEEP setting of the gas delivery apparatus is 15 cm H2O.

33-34. (canceled)

35. A method for controlling mechanical ventilation being provided to a patient, comprising a controller:
controlling a gas delivery system of a mechanical ventilator to deliver gas to the patient according to an FiO2 setting and a PEEP setting, wherein the FiO2 setting and the PEEP setting are adjustable;
controlling the delivery of the gas to the patient according to a first FiO2 value and a first PEEP value;
receiving signals representative of an oxygen concentration of the patient's blood from an oximetry sensor of the mechanical ventilator during the delivery of the gas to the patient, the oximetry sensor being coupled with the gas delivery system;
determining the oxygen concentration of the patient's blood based at least in part on the received signals,
based at least in part on the determined oxygen concentration of the patient's blood, controlling the gas delivery system to adjust the FiO2 setting to an updated FiO2 setting, and
based at least in part on the updated FiO2 setting, controlling the gas delivery apparatus to adjust the PEEP setting to an updated PEEP value.

36. The method of claim 35, comprising, based at least in part on the determined oxygen concentration of the patient's blood, controlling the gas delivery system to adjust the FiO2 setting to the updated FiO2 setting at least in part by actuating an oxygen source valve, and comprising, based at least in part on the updated FiO2 setting, controlling the gas delivery apparatus to adjust the PEEP setting to the updated PEEP value at least in part by actuating an exhalation valve.

37. (canceled)

38. A system for providing mechanical ventilation to a patient, comprising:
a gas delivery system for delivering gas to a patient, comprising:
an oximetry sensor for generating signals representative of an oxygen concentration of the patient's blood;
a mechanical gas mover;
an oxygen source;
a patient interface coupled with the mechanical gas mover and the oxygen source; and
a controller, coupled with the oximetry sensor and the compressor, for controlling the gas delivery system to deliver gas to the patient according to a FiO2 setting and a PEEP setting, wherein the FiO2 setting and the PEEP setting are configured to be adjustable, the controlling of the gas delivery system comprising:
receiving the signals representative of the oxygen concentration of the patient's blood from the oximetry sensor during the delivery of the gas to the patient,
determining the oxygen concentration of the patient's blood based at least in part on the received signals,
based at least in part on the determined oxygen concentration of the patient's blood, controlling the gas delivery system to adjust the FiO2 setting to an updated FiO2 setting, comprising actuating at least one oxygen source valve according to the updated FiO2 setting, the at least one oxygen source valve being coupled with, and for adjusting gas flow from, the oxygen source, and
based at least in part on the updated FiO2 setting, control the gas delivery system to adjust the PEEP setting to an updated PEEP setting, comprising actuating at least one exhalation valve according to the updated PEEP setting, the at least one exhalation valve being coupled with the compressor and the patient interface.

39-225. (canceled)
